(19) United States
(12) Patent Application Publication
(10) Pub. No.: US 2015/0306230 A1
(43) Pub. Date: Oct. 29, 2015

(54) DRUG DELIVERY VEHICLES COMPRISING CELLULOSE DERIVATIVES, STARCH DERIVATIVES, AND COMBINATIONS THEREOF

(71) Applicant: Celanese Acetate LLC, Irving, TX (US)

(72) Inventors: Michael Combs, Pembroke, VA (US); Wendy Bisset, Eggleston, VA (US); Lizbeth Milward, Blacksburg, VA (US); Adam Larkin, Dallas, TX (US); Naresh Budhavaram, Florence, KY (US)

(73) Assignee: Celanese Acetate LLC, Irving, TX (US)

(21) Appl. No.: 14/302,830

(22) Filed: Jun. 12, 2014

Related U.S. Application Data
(60) Provisional application No. 61/985,123, filed on Apr. 28, 2014.

(51) Int. Cl. A61K 47/38; A61L 26/00; A61K 9/70; A61K 47/18; A61K 47/24; A61K 47/32; A61K 47/22; A61K 9/00; A61K 47/14

(52) U.S. Cl. CPC A61K 47/38; A61K 9/0036; A61L 26/0014; A61L 26/0066; A61K 9/7084; A61K 47/14; A61K 47/24; A61K 47/32; A61K 47/22; A61K 47/18; A61L 2300/602

(57) ABSTRACT

A drug delivery vehicle (e.g., a patch, a pill, an intravaginal ring, and an implant) may include a polymer matrix comprising a plasticizer and at least one selected from the group consisting of: a cellulose ester, a cellulose ether, a starch ester, a starch ether, and a combination thereof. A drug may be dispersed in the polymer matrix, in another portion of the drug delivery vehicle, or both.

### BACKGROUND

The exemplary embodiments described herein relate to drug delivery vehicles, and methods relating thereto. As used herein, the term “vehicle” refers to a conveyance (e.g., a patch, a pill, an intravaginal ring, and an implant) for containing, transporting, and optionally releasing a desired compound. As used herein, the term “drug” refers generically to a compound that has a biological effect including active pharmaceutical agents, prodrugs of active pharmaceuticals, antifungal compounds, nutritional supplements, biological compounds like peptides, and so on.

It is often desirable and important to provide a sustained release of a drug to patients at a controlled rate, which may be administered in a variety of ways including pills for oral administration, patches for transdermal administration, and small rods for implantation. In some instances, drug delivery over a long period of time is preferred (e.g., for birth control). In other instances, delivery in a localized area for a relatively short period of time is preferred (e.g., for wound dressing and treatment). To accomplish this, different approaches have been used for controlled drug delivery to patients. For example, polymeric compositions may be loaded with drugs, which then diffuse into the patient at the application site. In many instances, the polymeric compositions may include polyurethanes, polyolefins, and ethylene vinyl acetate copolymers. These polymers are generally considered hydrophobic, which may limit the drug compositions and drug concentrations that may be used therewith due to hydrophilic/hydrophobic interactions. Therefore, compositions that provide hydrophilic polymeric matrices or may be blended with the hydrophobic polymers may be useful for increasing the types of drugs and concentrations of drugs that may be delivered by a controlled diffusion mechanism.

### BRIEF DESCRIPTION OF THE DRAWINGS

The following figures are included to illustrate certain aspects of the embodiments presented herein, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a cross-sectional diagram of a dermal patch.

FIG. 2 illustrates a cross-section of an intravaginal ring.

### DETAILED DESCRIPTION

The exemplary embodiments described herein relate to drug delivery vehicles that include plasticized cellulose derivatives, plasticized starch derivatives, or both, including methods relating thereto.

Plasticized cellulose derivatives and plasticized starch derivatives may be useful in forming the polymeric matrix of vehicles that deliver drugs to patients. Cellulose derivatives and starch derivatives have been shown to be plasticized with acetylsalicylic acid (aspirin) and other small molecules. In some instances, high concentrations of the small molecules can be achieved (e.g., 40:60 wt % cellulose derivative or starch derivative to acetylsalicylic acid), which may allow for high loading of a drug in a vehicle.

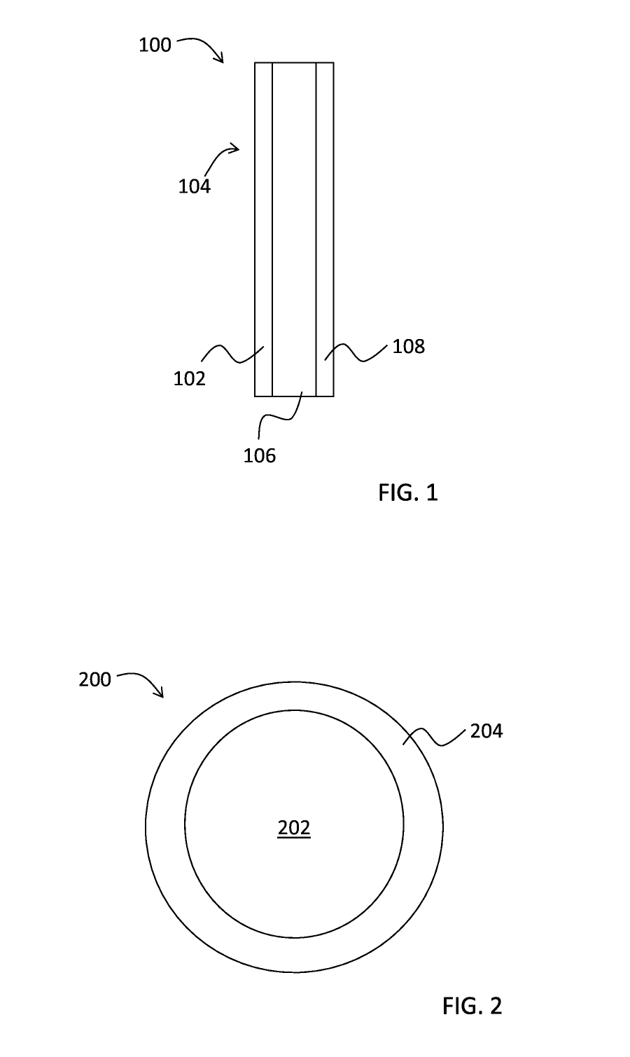

Further, plasticized cellulose derivatives and plasticized starch derivatives may formulated to be tacky or non-tacky at room temperature, which may allow for the use of plasticized cellulose derivatives and plasticized starch derivatives in various portions of a drug release vehicle. For example, referring to a cross-sectional diagram of a patch illustrated in FIG. 1, tacky compositions may be used as an adhesive 102 for the skin side 104 of the dermal patch 100 where the adhesive may contain a desired drug like nicotine or aspirin. Alternatively, or in combination with such an adhesive, a non-tacky (or less tacky) composition may be used as an intermediate layer 106 between the adhesive and a backing 108 of the dermal patch 100.

Additionally, plasticized cellulose derivatives and plasticized starch derivatives may be compounded with other polymers traditionally used in the production of drug delivery vehicles like ethylene vinyl acetate. Therefore, the compositions of such blends may be used to tailor the release profile of drug delivery vehicles produced therewith.

It should be noted that when “about” is used in reference to a number in a numerical list, the term “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit. Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments described herein. At the very least, and not as an attempt to limit the application of the doctrine of equivalents to the scope of the claim, each numerical parameter should at least be construed in light of the number of reported significant digits and by applying ordinary rounding techniques.

A drug delivery vehicle described herein may include a polymeric matrix that comprises plasticized cellulose derivatives, plasticized starch derivatives, or both and optionally further comprises other polymers (e.g., ethylene vinyl acetate copolymer and polyurethanes). In some instances, a polymeric matrix may further include one or more drugs. In some instances, a plasticizer of the cellulose derivatives or starch derivatives may be a drug.

As used herein, the term “plasticized cellulose derivatives” refers to cellulose derivatives having at least 1% plasticizer by weight of the cellulose derivatives. Cellulose derivatives suitable for use in conjunction with a polymeric matrix described herein may, in some embodiments, have ester substituents that include, but are not limited to, C₁-C₂₀ aliphatic esters (e.g., acetate, propionate, or butyrate), functional C₁-C₂₀ aliphatic esters (e.g., acrylates or diesters) aromatic esters (e.g., benzoate or phthalate), substituted aromatic esters, and the like, any derivative thereof, and any combination thereof. Cellulose derivatives suitable for use in conjunction with a polymeric matrix described herein may, in some embodiments, have ether substituents that include, but are not limited to, methylether, ethylether, propylether, hydroxypropylether, hydroxyethylether, hydroxyethyl methylether, hydroxypropyl methylether, carboxymethylether, and the like, and any combination thereof. In some instances, the cellulose derivatives described herein may have one or more ester substituents and one or more ether substituents.

In some embodiments, the cellulose derivatives may be bio-derived where not only the cellulose source is from a biological source, but also the acid or other reactants used to derivatize the cellulose. For example, acetic anhydride can be produced from a bio-derived acetic acid. As used herein, the term “bio-derived” refers to a compound or portion thereof originating from a biological source or produced via a biological reaction.

Cellulose derivatives suitable for use in conjunction with a polymeric matrix described herein may, in some embodiments, have a degree of substitution of the substituent ranging from a lower limit of about 0.5, 1.2, or 2 to an upper limit of about 3, 2.9, 2.7, or 2.5, and wherein the degree of substitution may range from any lower limit to any upper limit and encompass any subset therebetween.

Cellulose derivatives suitable for use in conjunction with a polymeric matrix described herein may, in some embodiments, have a molecular weight ranging from a lower limit of about 10,000, 15,000, 25,000, 50,000, or 85,000 to an upper limit of about 300,000, 200,000, 150,000, 125,000, 100,000, or 85,000, and wherein the molecular weight may range from any lower limit to any upper limit and encompass any subset therebetween. As used herein, the term “molecular weight” refers to a polystyrene equivalent number average molecular weight (“Mₙ”).

In some embodiments, cellulose derivatives described herein may have an intrinsic viscosity ranging from a lower limit of about 0.5 dL/g, 0.7 dL/g, or 1.0 dL/g to an upper limit of about 2.0 dL/g, 1.7 dL/g, 1.5 dL/g, or 1.3 dL/g, and wherein the intrinsic viscosity may range from any lower limit to any upper limit and encompass any subset therebetween. Intrinsic viscosity may be measured by forming a solution of 0.20 g/dL cellulose ester in 98/2 wt/wt acetone/water and measuring the flow times of the solution and the solvent at 30° C. in a #25 Cannon-Ubbelohde viscometer. Then, the modified Baker-Philippoff equation may be used to determine intrinsic viscosity (“IV”), which for this solvent system is Equation 1.

$$IV = ( \frac{k}{c} ) ( antilog ( ( log {n}_{rel} ) / k ) - 1 ) \qquad Equation 1$$

where

$${n}_{rel} = ( \frac{{t}_{1}}{{t}_{2}} ) ,$$

t₁=the average flow time of solution (having cellulose ester) in seconds, t₂=the average flow times of solvent in seconds, k=solvent constant (10 for 98/2 wt/wt acetone/water), and c=concentration (0.200 g/dL).

Cellulose derivatives suitable for use in conjunction with a polymeric matrix described herein may be derived from any suitable cellulosic source. Suitable cellulosic sources may, in some embodiments, include, but are not limited to, softwoods, hardwoods, cotton linters, switchgrass, bamboo, bagasse, industrial hemp, willow, poplar, perennial grasses (e.g., grasses of the Miscanthus family), bacterial cellulose, seed hulls (e.g., soy beans), kudzu, and the like, and any combination thereof. Further, it has been surprisingly discovered that the clarity of compositions described herein does not appear to be substantially impacted by the cellulosic source from which the cellulose esters are derived. This is unexpected because some existing cellulose ester products that require high quality use expensive cellulosic sources (e.g., hardwoods with low hemicellulose content) to achieve high clarity.

In some embodiments, the cellulose derivatives may be present in a polymeric matrix described herein in an amount ranging from a lower limit of about 0%_{,} 1%_{,} 5%, 10%, 25%, or 50% by weight of the polymeric matrix to an upper limit of about 80%, 75%, 50%, 25%, or 10% by weight of the polymeric matrix, and wherein the amount may range from any lower limit to any upper limit and encompass any subset therebetween.

As used herein, the term “plasticized starch derivatives” refers to starch derivatives having at least 1% plasticizer by weight of the starch derivatives. As used herein, the term “starch” refers to a natural polysaccharide that includes amylose and amylopectin in various ratios and derivatives thereof. Example of starches may include, but are not limited to, waxy starches, modified starches, native starches, dextrins, and maltodextrins with dextrose equivalents of 1-50. In some embodiments, the starch and starch derivatives described herein may have an amylose content of about 30% or less, 25% or less, or 10% or less. In some instances, the starch and starch derivatives described herein may have an amylose content of about 1% or less.

Starch derivatives suitable for use in conjunction with a polymeric matrix described herein may be derived from any suitable starch source. Native, modified, waxy, modified waxy, and hydrolyzed starches can be used. Suitable starch sources may, in some embodiments, include, but are not limited to, cereals, rice, wheat, maize, root vegetables, potatoes, corn, tapioca, cassava, acorns, arrowroot, arracacha, bananas, barley, breadfruit, buckwheat, canna, colacasia, katakuri, kudzu, malanga, millet, oats, oca, Polynesian arrowroot, sago, sorghum, sweet potatoes, rye, taro, chestnuts, water chestnuts, yams, beans, favas, lentils, mung beans, peas, chickpeas, and the like, and any combination thereof.

Starch derivatives may, in some embodiments, have ester substituents that include, but are not limited to, C₁-C₂₀ aliphatic esters (e.g., acetate, propionate, or butyrate), functional C₁-C₂₀ aliphatic esters (e.g., acrylates or diesters), aromatic esters (e.g., benzoate or phthalate), substituted aromatic esters, and the like, any derivative thereof, and any combination thereof. Starch derivatives may, in some embodiments, have ether substituents that include, but are not limited to, methylether, ethylether, propylether, hydroxypropylether, hydroxyethylether, hydroxyethyl methylether, hydroxypropyl methylether, carboxymethylether, and the like, and any combination thereof. In some instances, the starch derivatives described herein may have one or more ester substituents and one or more ether substituents.

In some embodiments, the starch derivatives may be bio-derived where not only the starch source is from a biological source, but also the acid or other reactants used to derivatize the starch. For example, acetic anhydride can be produced from a bio-derived acetic acid.

Starch derivatives suitable for use in conjunction with a polymeric matrix described herein may, in some embodiments, have a degree of substitution of the substituent ranging from a lower limit of about 0.5, 1.2, or 2 to an upper limit of about 3, 2.9, 2.7, or 2.5, and wherein the degree of substitution may range from any lower limit to any upper limit and encompass any subset therebetween.

Starch derivatives suitable for use in conjunction with a polymeric matrix described herein may, in some embodiments, have a molecular weight ranging from a lower limit of about 1,000, 15,000, or 25,000 to an upper limit of about 80,000, 50,000, or 30,000, and wherein the molecular weight may range from any lower limit to any upper limit and encompass any subset therebetween.

In some embodiments, the starch derivatives may be present in a polymeric matrix described herein in an amount ranging from a lower limit of about 0%, 1%, 5%, 10%, 25%, or 50% by weight of the polymeric matrix to an upper limit of about 80%, 75%, 50%, 25%, or 10% by weight of the polymeric matrix, and wherein the amount may range from any lower limit to any upper limit and encompass any subset therebetween.

Plasticizers suitable for use in conjunction with the cellulose derivatives and starch derivatives described herein may, in some embodiments, include, but are not limited to,

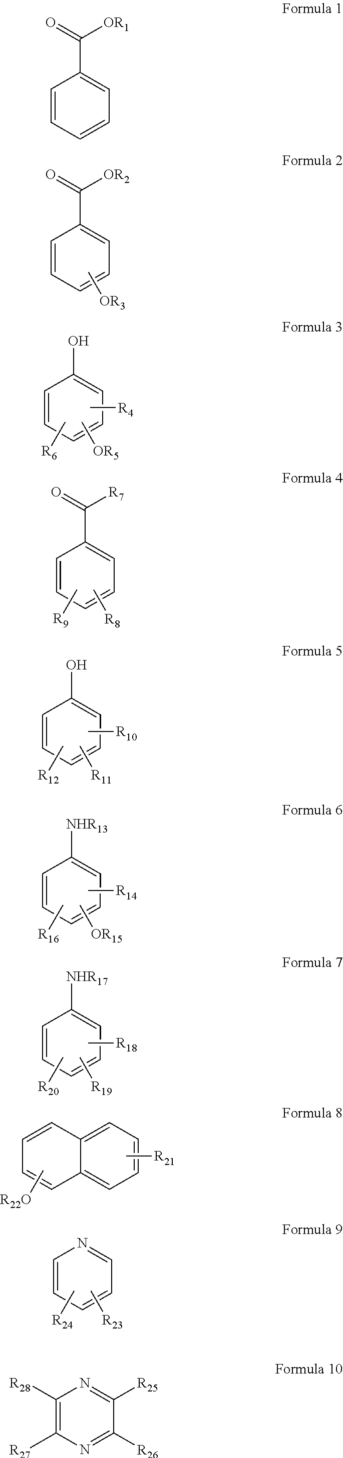

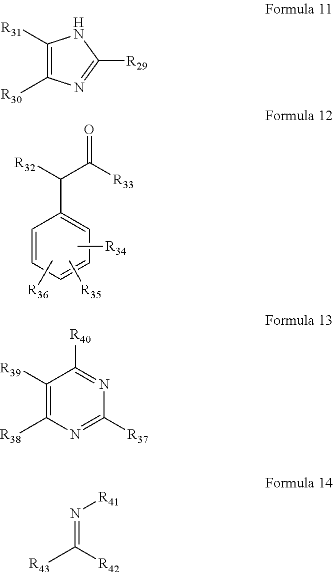

Formula 1 wherein R1 is H, C₁-C₄ alkyl, aryl, or C₁-C₄ alkyl aryl; Formula 2 wherein R2 is H, C₁-C₄ alkyl, aryl, or C₁-C₄ alkyl aryl and R3 is H, C₁-C₄ alkyl, aryl, C₁-C₄ alkyl aryl, acyl, or C₁-C₄ alkyl acyl; Formula 3 wherein R4 and R6 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide and R5 is H, C₁-C₄ alkyl, aryl, C₁-C₄ alkyl aryl, acyl, or C₁-C₄ alkyl acyl; Formula 4 wherein R7 is H, C₁-C₄ alkyl, aryl, C₁-C₄ alkyl aryl, OH, C₁-C₄ alkoxy, amine, or C₁-C₄ alkyl amine and R8 and R9 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; Formula 5 wherein R10, R11, and R12 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; Formula 6 wherein R13 is H, C₁-C₄ alkyl, aryl, or C₁-C₄ alkyl aryl, R14 and R16 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide, and R15 is H, C₁-C₄ alkyl, aryl, C₁-C₄ alkyl aryl, acyl, or C₁-C₄ alkyl acyl; Formula 7 wherein R17 is H or C₁-C₄ alkyl and R18, R19, and R20 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; Formula 8 wherein R21 is H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide and R22 is H, C₁-C₄ alkyl, aryl, C₁-C₄ alkyl aryl, acyl, C₁-C₄ alkyl acyl, amine, or C₁-C₄ alkyl amine; Formula 9 wherein R23 and R24 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; Formula 10 wherein R25, R26, R27, and R28 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; Formula 11 wherein R29, R30, and R31 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; Formula 12 wherein R32 is H, C₁-C₄ alkyl, aryl, C₁-C₄ alkyl aryl, R33 is H, C₁-C₄ alkyl, aryl, C₁-C₄ alkyl aryl, OH, C₁-C₄ alkoxy, acyl, C₁-C₄ alkyl acyl, amine, or C₁-C₄ alkyl amine, and R34, R35, and R36 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; Formula 13 wherein R37, R38, R39, and R40 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; Formula 14 wherein R41 is H, C₁-C₄ alkyl, aryl, C₁-C₄ alkyl aryl, OH, or C₁-C₄ alkoxy and R42 and R43 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; triazine (1,2,3, 1,2,4, or 1,3,5) with R substituents from each of the cyclic carbons or cyclic nitrogens that are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; triazole (1,2,3 or 1,2,4) with R substituents from each of the cyclic carbons or cyclic nitrogens that are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide; pyrrole with R substituents from each of the cyclic carbons or cyclic nitrogens that are independently H, C₁-C₄ alkyl, aryl, C₁-C₄ alkyl aryl, OH, C₁-C₄ alkoxy, COOH, C₁-C₄ alkyl carboxylate, acyl, C₁-C₄ alkyl acyl, amine, C₁-C₄ alkyl amine, amide, or C₁-C₄ alkyl amide; piperidine with R substituents from each of the cyclic carbons or cyclic nitrogens that are independently H, C₁-C₄ alkyl, aryl, C₁-C₄ alkyl aryl, OH, C₁-C₄ alkoxy, COOH, C₁-C₄ alkyl carboxylate, acyl, C₁-C₄ alkyl acyl, amine, C₁-C₄ alkyl amine, amide, or C₁-C₄ alkyl amide; piperazine with R substituents from each of the cyclic carbons or cyclic nitrogens that are independently H, C₁-C₄ alkyl, aryl, C₁-C₄ alkyl aryl, OH, C₁-C₄ alkoxy, COOH, C₁-C₄ alkyl carboxylate, acyl, C₁-C₄ alkyl acyl, amine, C₁-C₄ alkyl amine, amide, or C₁-C₄ alkyl amide; R44HN—R45-NHR46 where R44 and R46 are independently H, C₁-C₄ alkyl, aryl, C₁-C₄ alkyl aryl, COOH, C₁-C₄ alkyl carboxylate, acyl, C₁-C₄ alkyl acyl, amine, C₁-C₄ alkyl amine, amide, or C₁-C₄ alkyl amide and R45 is C₁-C₁₀ alkyl; and combinations thereof. As used herein, “alkyl” refers to a substituent with C and H that may be linear or branched (e.g., t-butyl) and saturated or unsaturated. As used herein, “aryl” refers to an aromatic ring that may include phenyl, naphthyl, and aromatic rings with heteroatoms.

Examples of plasticizers suitable for use in conjunction with the cellulose derivatives and starch derivatives described herein may, in some embodiments, include, but are not limited to, triacetin, trimethyl phosphate, triethyl phosphate, tributyl phosphate, triphenyl phosphate, triethyl citrate, acetyl trimethyl citrate, acetyl triethyl citrate, acetyl tributyl citrate, tributyl-o-acetyl citrate, dibutyl phthalate, diaryl phthalate, diethyl phthalate, dimethyl phthalate, di-2-methoxyethyl phthalate, di-octyl phthalate (and isomers), dibutyl tartrate, ethyl o-benzoylbenzoate, ethyl phthalyl ethyl glycolate, methyl phthalyl ethyl glycolate, n-ethyltoluenesulfonamide, o-cresyl p-toluenesulfonate, aromatic diol, substituted aromatic diols, aromatic ethers, tripropionin, polycaprolactone, glycerin, glycerin esters, diacetin, polyethylene glycol, polyethylene glycol esters, polyethylene glycol diesters, di-2-ethylhexyl polyethylene glycol ester, glycerol esters, diethylene glycol, polypropylene glycol, polyglycoldiglycidyl ethers, dimethyl sulfoxide, N-methyl pyrollidinone, propylene carbonate, C₁-C₂₀ dicarboxylic acid esters, dimethyl adipate (and other dialkyl esters), di-butyl maleate, di-octyl maleate, resorcinol monoacetate, catechol, catechol esters, phenols, epoxidized soy bean oil, castor oil, linseed oil, epoxidized linseed oil, other vegetable oils, other seed oils, difunctional glycidyl ether based on polyethylene glycol, alkyl lactones (e.g., γ-valerolactone), alkylphosphate esters, aryl phosphate esters, phospholipids, aromas (including some described herein, e.g., eugenol, cinnamyl alcohol, camphor, methoxy hydroxy acetophenone (acetovanillone), vanillin, and ethylvanillin), 2-phenoxyethanol, glycol ethers, glycol esters, glycol ester ethers, polyglycol ethers, polyglycol esters, ethylene glycol ethers, propylene glycol ethers, ethylene glycol esters (e.g., ethylene glycol diacetate), propylene glycol esters, polypropylene glycol esters, acetylsalicylic acid, acetaminophen, naproxen, imidazole, triethanol amine, benzoic acid, benzyl benzoate, salicylic acid, 4-hydroxybenzoic acid, propyl-4-hydroxybeonzoate, methyl-4-hydroxybeonzoate, ethyl-4-hydroxybeonzoate, benzyl-4-hydroxybeonzoate, butylated hydroxytoluene, butylated hydroxyanisol, sorbitol, xylitol, ethylene diamine, piperidine, piperazine, hexamethylene diamine, triazine, triazole, pyrrole, and the like, any derivative thereof, and any combination thereof.

Additional examples of plasticizers suitable for use in conjunction with the the cellulose derivatives and starch derivatives described herein may, in some embodiments, be nonionic surfactants that include, but are not limited to, polysorbates (e.g., TWEEN®20 or TWEEN®80, available from SigmaAldrich), sorbitan esters (e.g., SPAN@ products available from SigmaAldrich), polyethoxylated aromatic hydrocarbons (e.g., TRITON@ products available from SigmaAldrich), polyethoxylated fatty acids, polyethoxylated fatty alcohols (e.g., BRIJ@ products available from SigmaAldrich), fluorosurfactants, glucosides, and other nonionic surfactants with hydrocarbon tails (e.g., C₆-C₂₂ alkyl groups) and hydrophilic head groups with hydroxyl and ester groups, and combinations thereof. It has been discovered that some nonionic surfactants plasticize cellulose esters, alone or in combination with small molecule plasticizers. This is unexpected because traditional plasticizers are small molecules. By contrast, nonionic surfactants are bulky with long hydrocarbon tail groups and potentially large head groups. For example, polyoxyethylene (20) sorbitan monolaurate, which is significantly larger than traditional cellulose ester plasticizers like triacetin, has been observed to plasticize cellulose ester.

In some embodiments, the plasticizers may be food-grade plasticizers, which may be useful in producing compositions described herein for use in applications where the compositions may directly or indirectly contact food (e.g., food containers). As used herein, the term “food-grade” refers to a material that has been approved for contacting (directly or indirectly) food, which may be classified as based on the material's conformity to the requirements of the United States Pharmacopeia (“USP-grade”), the National Formulary (“NF-grade”), and/or the Food Chemicals Codex (“FCC-grade”). Examples of food-grade plasticizers may, in some embodiments, include, but are not limited to, triacetin, diacetin, tripropionin, trimethyl citrate, triethyl citrate, tributyl citrate, eugenol, cinnamyl alcohol, alkyl lactones (e.g., γ-valerolactone), methoxy hydroxy acetophenone (acetovanillone), vanillin, ethylvanillin, polyethylene glycols, 2-phenoxyethanol, glycol ethers, ethylene glycol ethers, propylene glycol ethers, polysorbate surfactants, sorbitan ester surfactants, polyethoxylated aromatic hydrocarbons, polyethoxylated fatty acids, polyethoxylated fatty alcohols, and the like, and any combination thereof.

In some embodiments, the plasticizers may be bio-derived, which may be useful in producing compositions that are bio-derived. For example, bio-derived triacetin, diacetin, tripropionin, glyceryl esters, may be produced from glycerol that is a byproduct of biodiesel. Other examples of plasticizers that may be bio-derived may include, but are not limited to, vanillin, acetovanillone, γ-valerolactone, eugenol, epoxidized soybean oil, castor oil, linseed oil, epoxidized linseed oil, and dicarboxylic esters (e.g., dimethyl adipate, dibutyl maleate). In some instances, aroma plasticizers may be extracts from natural products, and therefore, bio-derived plasticizers.

In some embodiments, the plasticizers may be semi-volatile to volatile plasticizers. Examples of some preferred semi-volatile to volatile plasticizers may include, but are not limited to, glycerol esters, (e.g., triacetin, diacetin, monoacetin), ethylene glycol diacetate, alkyl lactones (e.g., γ-valerolactone), dibutyl maleate, di-octyl maleate, dibutyl tartrate, eugenol, tributyl phosphate, tributyl-o-acetyl citrate, and resorcinol monoacetate.

In some instances, two or more plasticizers may be used with cellulose derivatives, starch derivatives, or both. In some instances, it has been surprisingly observed that two or more plasticizers may have synergistic effects. For the same total weight percent of total plasticizer, a cellulose ester in combination with multiple plasticizers may have a greater melt flow index than a cellulose ester in combination with the individual plasticizers alone, which is an unexpected observation.

In some embodiments, a polymeric matrix described herein may include plasticized cellulose derivatives, plasticized starch derivatives, or both at a total of about 1% to about 99% by weight of the polymeric matrix and an additional polymer at about 99% to about 1% by weight of the polymeric matrix. Additional polymers that may be blended with plasticized cellulose derivatives, plasticized starch derivatives, or both to form a polymeric matrix may include, but are not limited to, a polyolefin (e.g., polyethylene and polypropylene), ethylene copolymers (e.g., polymers that comprise ethylene monomers and at least one monomer of vinyl acetate, methyl acrylate, ethyl acrylate, n-butyl acrylate, ethyl methacrylate, acrylic acid, methacrylic acid, propylene, 1-butene, 1-pentene, 1-hexene, 1-heptene, 1-octene, 4-methyl-1-pentene, any derivative thereof, and any combination thereof), a thermoplastic polyurethane, an acrylic acid polymer, polytetrafluoroethylene (“PTFE”), an ethylene vinyl acetate copolymer derivative, a polyester, a polysiloxane, polybutadiene, polyisoprene, poly(methacrylate), poly(methyl methacrylate), a styrene-butadiene-styrene block copolymer, poly(hydroxyethylmethacrylate) (pHEMA), poly(vinyl chloride), poly(vinyl acetate), a polyether, a polyacrylonitrile, a polyethylene glycol, polymethylpentene, polybutadiene, polyhydroxy alkanoates, poly(lactic acid), poly(glycolic acid), acrylic acid-based polymers, a methacrylic acid based polymer, a polyanhydride, a polyorthoester, cross-linked poly(vinyl alcohol), neoprene rubber, butyl rubber, polyvinyl acetate phthalate, polyvinyl acetate, shellac, zein, polyethylene oxide (“PEO”), ethylene oxide-propylene oxide copolymers (include block copolymers like PLURONICS® (polyethylene oxide-polypropylene oxide-polyethylene oxide triblock polymers, available from BASF)), polyethylene-polypropylene glycol (e.g., poloxamer), carbomer, polycarbophil, chitosan, polyvinyl pyrrolidone (“PVP”), poly(vinyl alcohol) (“PVOH”), a polyethyleneimine, a polyacrylate, a polyacrylamide, a polymethacrylamide, a polyphosphazine, a polyoxazolidine, a polyhydroxyalkylcarboxylic acid, an alginic acid (e.g., carrageenate alginates, ammonium alginate, and sodium alginate), natural gums (e.g., gum guar, gum acacia, gum tragacanth, karaya gum, and gum xanthan), povidone, gelatin, and the like, any derivative thereof, any copolymer thereof, any blend polymer thereof, and combinations thereof.

In some instances, the additional polymers blended plasticized cellulose derivatives, plasticized starch derivatives, or both to form a polymeric matrix may be sufficiently hydrophobic that a compatibilizer is needed to produce a homogeneous blend. Exemplary compatibilizers for use in conjunction with such blend may be nonionic surfactants that include, but are not limited to, polysorbates (e.g., TWEEN®20 or TWEEN®80, available from SigmaAldrich), sorbitan esters (e.g., SPAN® products available from SigmaAldrich), polyethoxylated aromatic hydrocarbons (e.g., TRITON@ products available from SigmaAldrich), polyethoxylated fatty acids, polyethoxylated fatty alcohols (e.g., BRIJ@ products available from SigmaAldrich), fluorosurfactants, glucosides, and other nonionic surfactants with hydrocarbon tails (e.g., C₆-C₂₂ alkyl groups) and hydrophilic head groups with hydroxyl and ester groups, and combinations thereof. Additional exemplary compatibilizers may be polymers that include, but are not limited to, polyethylene glycol less than about 2000 molecular weight. Combinations of the foregoing may also be used. In some embodiments, compatibilizers may be present in the polymeric matrix in an amount of about 0.5% to about 20% by weight of the polymeric matrix. In some instances, the compatibilizer may also plasticize the starch derivatives or cellulose derivatives in the polymeric matrix.

In some instances, a polymeric matrix of a drug deliver vehicle described herein may further include additives. Examples of additives suitable for use in conjunction with a polymeric matrix described herein may, in some embodiments, include, but are not limited to, tackifiers, waxes, fillers, antioxidants, preservatives, viscosity modifiers, lubricants, softening agents, pigments, aromas, adhesion promoters (e.g., silanes and alkyl silanes), dehydrators, plasticizers that plasticize a component of the polymeric matrix described herein other than the cellulose derivatives and the starch derivatives, and the like, and combinations thereof.

In some embodiments, an additive may be present in a polymeric matrix described herein in an amount ranging from a lower limit of 0%, about 1%, 5%, 10%, or 20% by weight of the polymeric matrix to an upper limit of about 40%, 30%, or 20% by weight of the polymeric matrix, and wherein the amount of additive may range from any lower limit to any upper limit and encompass any subset therebetween.

Tackifiers may be useful in increasing the tack of a polymeric matrix, which may be useful in adhesive portions of a drug delivery vehicle. Examples of tackifiers suitable for use in conjunction with a polymeric matrix described herein may, in some embodiments, include, but are not limited to, methylcellulose, ethylcellulose, hydroxyethylcellulose, carboxy methylcellulose, carboxy ethylcellulose, amides, diamines, polyesters, polycarbonates, silyl-modified polyamide compounds, polycarbamates, urethanes, natural resins, natural rosins, rosin esters (SYLVATAC® RE85 and SYLVALITE® RE100, both esters of tall oil rosin, available from Arizona Chemical), shellacs, acrylic acid polymers, 2-ethylhexylacrylate, acrylic acid ester polymers, acrylic acid derivative polymers, acrylic acid homopolymers, anacrylic acid ester homopolymers, poly(methyl acrylate), poly(butyl acrylate), poly(2-ethylhexyl acrylate), acrylic acid ester co-polymers, methacrylic acid derivative polymers, methacrylic acid homopolymers, methacrylic acid ester homopolymers, poly(methyl methacrylate), poly(butyl methacrylate), poly(2-ethylhexyl methacrylate), acrylamido-methyl-propane sulfonate polymers, acrylamido-methyl-propane sulfonate derivative polymers, acrylamido-methyl-propane sulfonate co-polymers, acrylic acid/acrylamido-methyl-propane sulfonate co-polymers, benzyl coco di-(hydroxyethyl) quaternary amines, p-T-amyl-phenols condensed with formaldehyde, dialkyl amino alkyl (meth)acrylates, acrylamides, N-(dialkyl amino alkyl) acrylamide, methacrylamides, hydroxy alkyl (meth)acrylates, methacrylic acids, acrylic acids, hydroxyethyl acrylates, ethylene vinyl acetate, vinyl acetate ethylene polymers, aliphatic hydrocarbons, cycloaliphatic hydrocarbons (e.g., EASTOTAC® products, available from Eastman Chemical Co.), aromatic hydrocarbons, aromatically modified aliphatic hydrocarbons, cycloaliphatic hydrocarbons, hydrogenated versions of the foregoing hydrocarbons, terpenes, polyterpenes, modified terpenes (e.g., phenolic modified terpene resins like SYLVARES™ TP96 and SYLVARES™ TP2040, available from Arizona Chemical), and the like, any derivative thereof, and any combination thereof.

In some embodiments, tackifiers suitable for use in conjunction with a polymeric matrix described herein may be food-grade tackifiers. Examples of food-grade tackifiers may, in some embodiments, include, but are not limited to, methylcellulose, ethylcellulose, hydroxyethylcellulose, carboxy methylcellulose, carboxy ethylcellulose, natural resins, natural rosins, and the like, and any combination thereof.

In some embodiments, compatibilizers may be used to more homogeneously incorporate tackifiers into a polymer matrix described herein. Suitable compatibilizers may include those described above relative to the base polymer composition and may be used at similar concentrations.

In some embodiments, the plasticized cellulose derivatives, plasticized starch derivatives, or both in a polymeric matrix may provide sufficient tack such that little to no additional tackifying resins (e.g., about 5% or less weight of the polymeric matrix) are required to achieve a tacky polymeric matrix.

Waxes may be useful, in some instances, to decrease the tack or increase the flowability of a polymeric matrix described herein. In some embodiments, waxes may have a melting temperature of about 45° C. to about 125° C. Examples of waxes suitable for use in conjunction with a polymeric matrix described herein may, in some embodiments, include, but are not limited to, paraffin waxes (e.g., PACEMAKER@ products, available from Citgo Petroleum, OKERIN® products, available from Astor Wax Corporation, PENRECO® products, available from Pennzoil Products Co, R-7152 products, available from Moore & Munger, and PARAFIN WAX 1297, available from International Waxes Ltd.), microcrystalline waxes (e.g., VICTORY@ AMBER WAX, available from Petrolite Corp, BARECO® ES-796 Amber Wax, available from Bareco, and OKERIN® 177, available from Astro Wax Corporation), polyethylene waxes (e.g., POLYWAX® products, available from Petrolite, Inc.), polypropylene waxes, by-product polyethylene waxes, Fischer-Tropsch waxes, and the like, and combinations thereof.

In some embodiments, compatibilizers may be used to more homogeneously incorporate waxes into a polymer matrix described herein. Suitable compatibilizers may include those described above relative to the base polymer composition and may be used at similar concentrations.

Fillers may, in some embodiments, increase the rigidity and decrease the creep of a polymeric matrix described herein, which may consequently increase the mechanical rigidity of an article produced therewith. Examples of fillers may include, but are not limited to, coconut shell flour, walnut shell flour, wood flour, wheat flour, soybean flour, gums, protein materials, calcium carbonate, talc, zeolite, clay, rigid compounds (e.g. lignin), thickeners, unreacted starches, modified starches (e.g., with modifications other than ester modifications like hydroxyethyl starch, hydrolyzed starch, cationic starch, starch phosphate, oxidized starch, and the like), waxy starches, cellulose nanofibrils, nanocrystalline cellulose, glass microspheres, carbonates, talc, silica, silicates, magnesium silicates, and the like, and any combination thereof.

In some embodiments, fillers suitable for use in conjunction with a polymeric matrix described herein may be food-grade fillers. Examples of food-grade fillers may, in some embodiments, include, but are not limited to, coconut shell flour, walnut shell flour, wood flour, wheat flour, soybean flour, gums, starches, protein materials, calcium carbonate, and the like, and any combination thereof.

Preservatives and antioxidants may be useful mitigating degradation of the polymer matrix or drug therein. For example, preservatives and antioxidant may be useful in mitigating oxidation of the drugs and bacterial growth.

Preservatives suitable for use in conjunction with a polymeric matrix described herein may, in some embodiments, include, but are not limited to, benzoates, parabens (e.g., the propyl-4-hydroxybeonzoate series), and the like, and any combination thereof.

Antioxidants suitable for use in conjunction with a polymeric matrix described herein may, in some embodiments, include, but are not limited to, anthocyanin, ascorbic acid, glutathione, lipoic acid, uric acid, resveratrol, flavonoids, carotenes (e.g., beta-carotene), carotenoids, tocopherols (e.g., alpha-tocopherol, beta-tocopherol, gamma-tocopherol, and delta-tocopherol), tocotrienols, tocopherol esters (e.g., tocopherol acetate), ubiquinol, gallic acids, melatonin, secondary aromatic amines, benzofuranones, hindered phenols, polyphenols, hindered amines, organophosphorus compounds, thioesters, benzoates, lactones, hydroxylamines, butylated hydroxytoluene (“BHT”), butylated hydroxyanisole (“BHA”), hydroquinone, and the like, and any combination thereof.

In some embodiments, antioxidants suitable for use in conjunction with a polymeric matrix described herein may be food-grade antioxidants. Examples of food-grade antioxidants may, in some embodiments, include, but are not limited to, ascorbic acid, vitamin A, tocopherols, tocopherol esters, beta-carotene, flavonoids, BHT, BHA, hydroquinone, and the like, and any combination thereof.

Viscosity modifiers may, in some embodiments, be advantageous in modifying the melt flow index of a polymeric matrix described herein and/or modify the viscosity of a polymeric matrix described herein. Viscosity modifiers suitable for use in conjunction with a polymeric matrix described herein may, in some embodiments, include, but are not limited to, polyethylene glycols, polypropylene glycols, glycerin, and the like, and any combination thereof, which, in some embodiments, may be a food-grade viscosity modifier.

Pigments and dyes suitable for use in conjunction with a polymeric matrix described herein may, in some embodiments, include, but are not limited to, plant dyes, vegetable dyes, titanium dioxide, silicon dioxide, tartrazine, E102, phthalocyanine blue, phthalocyanine green, quinacridones, perylene tetracarboxylic acid di-imides, dioxazines, perinones disazo pigments, anthraquinone pigments, carbon black, metal powders, iron oxide, ultramarine, calcium carbonate, kaolin clay, aluminum hydroxide, barium sulfate, zinc oxide, aluminum oxide, CARTASOL® dyes (cationic dyes, available from Clariant Services) in liquid and/or granular form (e.g., CARTASOL® Brilliant Yellow K-6G liquid, CARTASOL® Yellow K-4GL liquid, CARTASOL® Yellow K-GL liquid, CARTASOL® Orange K-3GL liquid, CARTASOL® Scarlet K-2GL liquid, CARTASOL® Red K-3BN liquid, CARTASOL® Blue K-5R liquid, CARTASOL® Blue K-RL liquid, CARTASOL® Turquoise K-RL liquid/granules, CARTASOL® Brown K-BL liquid), FASTUSOL® dyes (an auxochrome, available from BASF) (e.g., Yellow 3GL, Fastusol C Blue 74L), and the like, any derivative thereof, and any combination thereof.

In some embodiments, pigments and dyes suitable for use in conjunction with a polymeric matrix described herein may be food-grade pigments and dyes. Examples of food-grade pigments and dyes may, in some embodiments, include, but are not limited to, plant dyes, vegetable dyes, titanium dioxide, and the like, and any combination thereof.

Aromas suitable for use in conjunction with a polymeric matrix described herein may, in some embodiments, include, but are not limited to, spices, spice extracts, herb extracts, essential oils, smelling salts, volatile organic compounds, volatile small molecules, methyl formate, methyl acetate, methyl butyrate, ethyl acetate, ethyl butyrate, isoamyl acetate, pentyl butyrate, pentyl pentanoate, octyl acetate, myrcene, geraniol, nerol, citral, citronellal, citronellol, linalool, nerolidol, limonene, camphor, terpineol, alpha-ionone, thujone, benzaldehyde, eugenol, isoeugenol, cinnamaldehyde, ethyl maltol, vanilla, vannillin, cinnamyl alcohol, anisole, anethole, estragole, thymol, furaneol, methanol, rosemary, lavender, citrus, freesia, apricot blossoms, greens, peach, jasmine, rosewood, pine, thyme, oakmoss, musk, vetiver, myrrh, blackcurrant, bergamot, grapefruit, acacia, passiflora, sandalwood, tonka bean, mandarin, neroli, violet leaves, gardenia, red fruits, ylang-ylang, acacia farnesiana, mimosa, tonka bean, woods, ambergris, daffodil, hyacinth, narcissus, black currant bud, iris, raspberry, lily of the valley, sandalwood, vetiver, cedarwood, neroli, strawberry, carnation, oregano, honey, civet, heliotrope, caramel, coumarin, patchouli, dewberry, helonial, coriander, pimento berry, labdanum, cassie, aldehydes, orchid, amber, orris, tuberose, palmarosa, cinnamon, nutmeg, moss, styrax, pineapple, foxglove, tulip, wisteria, clematis, ambergris, gums, resins, civet, plum, castoreum, civet, myrrh, geranium, rose violet, jonquil, spicy carnation, galbanum, petitgrain, iris, honeysuckle, pepper, raspberry, benzoin, mango, coconut, hesperides, castoreum, osmanthus, mousse de chene, nectarine, mint, anise, cinnamon, orris, apricot, plumeria, marigold, rose otto, narcissus, tolu balsam, frankincense, amber, orange blossom, bourbon vetiver, opopanax, white musk, papaya, sugar candy, jackfruit, honeydew, lotus blossom, muguet, mulberry, absinthe, ginger, juniper berries, spicebush, peony, violet, lemon, lime, hibiscus, white rum, basil, lavender, balsamics, fo-ti-tieng, osmanthus, karo karunde, white orchid, calla lilies, white rose, rhubrum lily, tagetes, ambergris, ivy, grass, seringa, spearmint, clary sage, cottonwood, grapes, brimbelle, lotus, cyclamen, orchid, glycine, tiare flower, ginger lily, green osmanthus, passion flower, blue rose, bay rum, cassie, African tagetes, Anatolian rose, Auvergne narcissus, British broom, British broom chocolate, Bulgarian rose, Chinese patchouli, Chinese gardenia, Calabrian mandarin, Comoros Island tuberose, Ceylonese cardamom, Caribbean passion fruit, Damascena rose, Georgia peach, white Madonna lily, Egyptian jasmine, Egyptian marigold, Ethiopian civet, Farnesian cassie, Florentine iris, French jasmine, French jonquil, French hyacinth, Guinea oranges, Guyana wacapua, Grasse petitgrain, Grasse rose, Grasse tuberose, Haitian vetiver, Hawaiian pineapple, Israeli basil, Indian sandalwood, Indian Ocean vanilla, Italian bergamot, Italian iris, Jamaican pepper, May rose, Madagascar ylang-ylang, Madagascar vanilla, Moroccan jasmine, Moroccan rose, Moroccan oakmoss, Moroccan orange blossom, Mysore sandalwood, Oriental rose, Russian leather, Russian coriander, Sicilian mandarin, South African marigold, South American tonka bean, Singapore patchouli, Spanish orange blossom, Sicilian lime, Reunion Island vetiver, Turkish rose, Thai benzoin, Tunisian orange blossom, Yugoslavian oakmoss, Virginian cedarwood, Utah yarrow, West Indian rosewood, and the like, and any combination thereof.

In some embodiments, a polymeric matrix described herein may be at least in part bio-derived. In some embodiments, the amount of a polymeric matrix that is bio-derived may range from a lower limit of about 2%, 5%, 10%, 25%, 50%, 75%, or 90% by weight of the polymeric matrix to an upper limit of about 100%, 99%, 95%, 90%, 75%, or 50% by weight of the polymeric matrix, and wherein the amount of the polymeric matrix that is bio-derived may range from any lower limit to any upper limit and encompasses any subset therebetween.

In some instances, a component of a polymeric matrix described herein may perform more than one function. For example, BHT and BHA are both antioxidants and plasticizers for cellulose derivatives, starch derivatives, or both. Additionally, nonionic surfactants may, in some instances, function as both plasticizers and compatibilizers. In another example, aromas like eugenol, cinnamyl alcohol, camphor, methoxy hydroxy acetophenone (acetovanillone), vanillin, and ethylvanillin may also plasticize cellulose derivatives, starch derivatives, or both. In yet another example, benzoates and parabens (e.g., the propyl-4-hydroxybeonzoate series) may be both preservatives and plasticizers for cellulose derivatives, starch derivatives, or both. In another example, some plasticizers may also be drug (e.g., acetylsalicylic acid, acetaminophen, and naproxen).

Drugs that may be suitable use in conjunction with a drug delivery device described herein may, in some embodiments, be for the prevention, mitigation, and/or treatment of diseases, conditions, and/or symptoms thereof in a patient. Examples of diseases and conditions may include, but are not limited to, arthritis, rheumatoid arthritis, juvenile rheumatoid arthritis, psoriatic arthritis, osteoarthritis, gouty arthritis, refractory rheumatoid arthritis, chronic non-rheumatoid arthritis, osteoporosis/bone resorption, osteophorosis, ulcerative colitis, skin diseases, psoriasis, acne vulgaris, rosacea, dermatitis, contact dermatitis, eczema, delayed-type hypersensitivity in skin disorders, type I diabetes, type II diabetes, Alzheimer's disease, inflammatory disorders, immunodeficiency, inflammatory bowel disease, irritable bowel syndrome, Crohn's disease, diarrhea disease, antibiotic associated diarrhea, pediatric diarrhea, chronic constipation, heartburn, appendicitis, autoimmune disorders, multiple sclerosis, muscle degeneration, coeliac disease, diabetes mellitus, organ transplantation, bacterial infections, viral infections, fungal infections, periodontal disease, urogenital disease, sexually transmitted disease, HIV infection, HIV replication, HIV associated diarrhea, surgical associated trauma, surgical-induced metastatic disease, nausea, weight loss, weight gain, anorexia, bulimia, fever control, cachexia, wound healing, ulcers, gut barrier function, allergies, Hay Fever, allergic rhinitis, anaphylaxis, asthma, respiratory disorders, lung diseases, pulmonary fibrosis, chronic obstructive pulmonary disease, circulatory disorders, anemia, disorders of the blood coagulation system, renal disease, disorders of the central nervous system, hepatic disease, ischemia, nutritional disorders, endocrine disorders, epidermal disorders, multiple myeloma, uveititis, acute and chronic myelogenous leukemia, anti-clotting, coronary heart disease, vasculitis, ischemic heart disease, atherosclerosis, strokes, peripheral arterial disease, ischemic-induced cell damage, high blood cholesterol levels, high-density lipoprotein (HDL) levels, high blood pressure, pancreatic β cell destruction, rheumatoid spondylitis, adult respiratory distress syndrome (ARDS), bone resorption diseases, ischemia reperfusion injury, brain trauma, cerebral malaria, sepsis, septic shock, toxic shock syndrome, blood infection, fever, myalgias due to infection, HIV-1, HIV-2, HIV-3, immune system disorders, cytomegalovirus, colds, influenza, adenovirus, the herpes viruses (including HSV-1, HSV-2), herpes zoster infection, herpes simplex/cold sores, infections, disorders associated with C-reactive protein, myositis, lupus, Celiac disease, prostatitis, tumor, sexual dysfunction, inflammatory disease, thyroid diseases, pregnancy, headaches, acute pain, rashes, addiction, addiction to habit forming drugs, addiction to smoking, upper respiratory tract infection, neurodegenerative disease, dyslexia, dyspraxia, autism, Asperger's disease, mild cognitive impairment, poor concentration, attention deficit disorder (ADD), attention deficit hyperactive disorder (ADHD), depression, mood swings, bipolar disorders, cancer, leukemia, acute and chronic myelogenous leukemia, colon cancer, prostate cancer, kidney cancer, liver cancer, breast cancer, lung cancer, melanoma, brain cancer, cervical cancer, Hodgkin's lymphoma, non-Hodgkin's lymphoma, ovarian cancer, testicular cancer, thyroid cancer, uterine cancer, urinary tract infection, nervous system infection, and the like. In some instances, a drug delivery device described herein may be useful in the prevention, mitigation, and/or treatment of other diseases, conditions, and/or symptoms.

Examples of drugs that may be suitable use in conjunction with a drug delivery device described herein may include, but are not limited to, active pharmaceuticals, prodrugs of active pharmaceuticals, active biologicals, antibiotics, antifungals, antitoxins, antigens, therapeutics, preventive therapeutics, nutritional supplements, and combinations thereof. It should be noted that some drugs may overlap into two or more types or categories of drugs described herein. As used herein, the term “prodrugs of active pharmaceuticals” encompasses compounds that are inactive or less than active and can increase in activity upon reaction in the body (e.g., after hydrolysis), irradiation (e.g., radiation sensitizers), or the like.

Examples of active pharmaceuticals and prodrugs of active pharmaceuticals that may be suitable use in conjunction with a drug delivery vehicle described herein may include, but are not limited to, 16-alpha fluoroestradiol, 16-alpha-gitoxin, 16-epiestriol, 17-alpha dihydroequilenin, 17-alpha estradiol, 17-beta estradiol, 17-hydroxy progesterone, 1-alpha-hydroxyvitamin D2, 1-dodecpyrrolidinone, 20-epi-1,25 dihydroxyvitamin D3, 22-oxacalcitriol, 2CW, 2′-nor-cGMP, 3-isobutyl GABA, 5-ethynyluracil, 6-FUDCA, 7-methoxytacrine, abamectin, abanoquil, abcizimab (commercially available as REOPRO® from Eli Lilly and Company), abecarnil, abiraterone, ablukast, ablukast sodium, acadesine, acamprosate, acarbose, acebutolol, acecamide hydrochloride, aceclidine, aceclofenae, acedapsone, aceglutamide aluminum, acemannan, acetaminophen, acetazolamide, acetohexamide, acetohydroxamic acid, acetomepregenol, acetophenazine maleate, acetosulfone sodium, acetylcholine chloride, acetylcysteine, acetyl-L-carnitine, acetylmethadol, acifran, acipimox, acitemate, acitretin, acivicin, aclarubicin, aclatonium, acodazole hydrochloride, aconiazide, acrisorcin, acrivastine, acronine, actisomide, actodigin, acyclovir, acylfulvene, adafenoxate, adalimumab (commercially available as HUMIRA® from Abbott Laboratories), adapalene, adapalene, adatanserin, adatanserin hydrochloride, adecypenol, adecypenol, adefovir, adelmidrol, ademetionine, adenosine, adinazolam, adipheinine hydrochloride, adiposin, adozelesin, adrafinil, adrenalone, airbutamine, alacepril, alamecin, alanine, alaproclate, alaptide, albendazole, albolabrin, albuterol (commercially available as VENTOLIN® from GlaxoSmithKline), albutoin, alclofenae, alclometasone dipropionate, aluminum chlorhydroxyallantoinate (commercially available as ALCOLOXA® from TRI-K Industries, Inc.), aldecalmycin, aldesleukin, aldioxa, alendronate sodium (commercially available as FOSAMAX® from Merck), alendronic acid, alentemol, alentemol hydrobromide, aletamine hydrochloride, aleuronium chloride, alexidine, alfacalcidol, alfentanil hydrochloride, alfuzosin, algestone acetonide, alglucerase, aliflurane, alinastine, alipamide, allantoin, allobarbital, allopurinol, a tachy-kinins (TK) antagonist, alonimid, alosetron, alosetron hydrochloride, alovudine, alpertine, alpha amylase, alpha idosone, alpidem, alprazolam (commercially available as XANAX® from Pfizer, Inc.), alprenolol hydrochloride, alprenoxime hydrochloride, alprostadil, alrestatin sodium, altanserin tartrate, alteplase, althiazide, altretamine, altromycin B, alverinc citrate, alvircept sudotox, amadinone acetate, amantadine hydrochloride, ambamustine, ambomycin, ambruticin, ambuphylline, ambuside, amcinafal, amcinonide, amdinocillin, amdinocillin pivoxil, amedalin hydrochloride, amelometasone, ameltolide, amesergide, ametantrone acetate, amezinium metilsulfate, amfebutamone, amfenac sodium, amflutizole, amicycline, amidephrine mesylate, amidox, amifloxacin, amifostine, amikacin, amiloride hydrochloride, aminacrine hydrochloride, aminobenzoate potassium, aminobenzoate sodium, aminocaproic acid, aminoglutethimide, aminohippurate sodium, aminolevulinic acid, aminophylline, aminorex, aminosalicylate sodium, aminosalicylic acid, amiodarone, amiprilose hydrochloride, amiquinsin hydrochloride, amisulpride, amitraz, amitriptyline hydrochloride, amlexanox, amlodipine, amobarbital sodium, amodiaquine, amodiaquine hydrochloride, amorolfine, amoxapine, amoxicillin, amphecloral, amphetamine sulfate, amphomycin, amphotericin B, ampicillin, ampiroxicam, ampyzine sulfate, amquinate, amrinone, aminone, amrubicin, amsacrine, amythiamicin, anagestone acetate, anagrelide, anakinra, ananain, anaritide, anaritide acetate, anastrozole (commercially available as ARIMIDEX® from AstraZeneca), anazolene sodium, ancrod, andrographolide, androstenedione, angiogenesis inhibitors, angiotensin amide, anidoxime, anileridine, anilopam hydrochloride, aniracetam, anirolac, anisotropine methylbromide, anistreplase, anitrazafen, anordrin, antagonist D, antagonist G, antarelix, antazoline phosphate, anthelmycin, anthralin, anthramycin, antiandrogen, antihemophilic factor (commercially available as XYNTHA® from Pfizer, Inc.), acedapsone, felbamate, antiestrogen, antineoplaston, antipyrine, antisense oligonucleotides, apadoline, apafant, apalcillin sodium, apaxifylline, apazone, aphidicolin glycinate, apixifylline, apomorphine hydrochloride, apraclonidine, apraclonidine hydrochloride, apramycin, aprindine, aprindine hydrochloride, aprosulate sodium, aprotinin, aptazapine maleate, aptiganel, apurinic acid, apurinic acid, aranidipine, aranotin, arbaprostil, arbekicin, 1-methyl-2-((phenylthio)methyl)-3-carbethoxy-4-((dimethylamino)methyl)-5-hydroxy-6-bromindole (commercially available as ARBIDOL® from Masterlek), arbutamine hydrochloride, arclofenin, ardeparin sodium, (2R,4R)-1-[(2S)-5-(diaminomethylideneamino)-2-[[(3R)-3-methyl-1,2,3,4-tetrahydroquinolin-8-yl]sulfonylamino]pentanoyl]-4-methyl-piperidine-2-carboxylic acid (commercially available as ARGATROBAN® from GlaxoSmithKline), arginine, argipressin tannate, arildone, aripiprazol, arotinolol, arpinocid, arteflene, artilide fumarate, asimadoline, aspalatone, asparaginase, aspartic acid, aspartocin, asperfuran, aspirin, aspoxicillin, asprelin, astemizole, astromicin sulfate, asulacrine, atamestane, atenolol, atevirdine, atipamezole, atiprosin maleate, atolide, atorvastatin (commercially available as LIPITOR® from Pfizer, Inc.), atosiban, atovaquone, atpenin B, atracurium besylate, atrimustine, atrinositol, atropine, auranofin, aureobasidin A, aurothioglucose, avilamycin, avoparcin, avridine, nizatidine (commercially available as AXID® from GlaxoSmithKline), axinastatin 1, axinastatin 2, axinastatin 3, azabon, azacitidinie, azaclorzine hydrochloride, azaconazole, azadirachtine, azalanstat dihydrochloride, azaloxan fumarate, azanator maleate, azanidazole, azaperone, azaribine, azaserine, azasetron, azatadine maleate, azathioprine, azathioprine sodium, azatoxin, azatyrosine, azelaic acid, azelastine, azelnidipine, azepindole, azetepa, azimilide, azithromycin, azlocillin, azolimine, azosemide, azotomycin, aztreonam, azumolene sodium, bacampicillin hydrochloride, baccatin III, bacitracin, baclofen, bacoside A, bacoside B, bactobolamine, balanol, balazipone, balhimycin, balofloxacin, balsalazide, bambermycins, bambuterol, bamethan sulfate, bamifylline hydrochloride, bamidazole, baohuoside 1, barmastine, barnidipine, basifungin, batanopride hydrochloride, batebulast, batelapine maleate, batimastat, beauvericin, becanthone hydrochloride, becaplermin, becliconazole, beclomethasone dipropionate, befloxatone, beinserazide, belfosdil, belladonna, beloxamide, bemesetron, bemitradine, bemoradan, benapryzine hydrochloride, benazepril hydrochloride, benazeprilat, bendacalol mesylate, bendazac, bendroflumethiazide, benflumetol, benidipine, benorterone, benoxaprofen, benoxaprofen, benoxinate hydrochloride, benperidol, bentazepam, bentiromide, benurestat, benzbromarone, benzethonium chloride, benzetimide hydrochloride, benzilonium bromide, benzindopyrine hydrochloride, benzisoxazole, benzocaine, benzochlorins, benzoctamine hydrochloride, benzodepa, benzoidazoxan, benzonatate, benzoyl peroxide, benzoylpas calcium, benzoylstaurosporine, benzquinamide, benzthiazide, benztropine, benztropine mesylate, benzydamine hydrochloride, benzylpenicilloyl polylysine, bepridil, bepridil hydrochloride, beractant, beraprost, berefrine, berlafenone, bertosamil, berythromycin, besipirdine, beta-alethine, betaclamycin B, betamethasone, betamipron, betaxolol, betaxolol hydrochloride, bethanechol chloride, bethanidine sulfate, betulinic acid, bevacizumab (commercially available as AVASTIN® available from Genenetech), bevantolol, bevantolol hydrochloride, bezafibrate, bFGF inhibitor, bialamicol hydrochloride, biapenem, bicalutamide, bicifadine hydrochloride, biclodil hydrochloride, bidisomide, bifemelane, bifonazole, bimakalim, bimithil, bindarit, biniramycin, binospirone, bioxalomycin alpha2, bipenamol hydrochloride, biperiden, biphenamine hydrochloride, biriperone, bisantrene, bisaramil, bisaziridinylspermine, bis-benzimidazole A, bis-benzimidazole B, bisnafide, bisobrin lactate, bisoprolol, bispyrithione magsulfex, bistramide D, bistramide K, bistratene A, bithionolate sodium, bitolterol besylate, bivalirudin, bizelesin, bleomycin sulfate, bolandiol dipropionate, bolasterone, boldenone undecylenate, boldine, bolenol, bolmantalate, bopindolol, bosentan, boxidine, brefeldin, breflate, brequinar sodium, bretazenil, bretylium bosylate, brifentanil hydrochloride, brimonidine, brinolase, brocresine, brocrinat, brofoxine, bromadoline maleate, bromazepam, bromchlorenone, bromelains, bromfenac, brominidione, bromocriptine, bromodiphenhydramine hydrochloride, bromoxamide, bromperidol, bromperidol decanoate, brompheniramine baleate, broperamole, bropirimine, brotizolam, bucamide maleate, bucindolol, buclizine hydrochloride, bucromarone, budesonide (commercially available as RHINOCORT® and ENTOCORT® from AstraZeneca), budipine, budotitane, buformin, bumetamide, bunaprolast, bunazosin, bunolol hydrochloride, bupicomide, bupivacaine hydrochloride, buprenorphine hydrochloride, bupropion hydrochloride, buramate, buserelin acetate, buspirone hydrochloride, busulfan, butabarbital, butacetin, butaclamol hydrochloride, butalbital, butamben, butamirate citrate, butaperazine, butaprost, butedronate tetrasodium, butenafine, buterizine, buthionine sulfoximine, butikacin, butilfenin, butirosin sulfate, butixirate, butixocort propionate, butoconazole nitrate, butonate, butopamine, butoprozine hydrochloride, butorphanol, butoxamine hydrochloride, butriptyline hydrochloride, cactinomycin, cadexomer iodine, caffeine, calanolide A, calcifediol, calcipotriene, calcipotriol, calcitonin, calcitriol, calcium undecylenate, calphostin C, calusterone, cambendazole, camonagrel, camptothecin derivatives, canarypox IL-2, candesartan, candicidin, candoxatril, candoxatrilat, caniglibose, canrenoate potassium, canrenone, capecitabine, capobenate sodium, capobenic acid, capreomycin sulfate, capromab, capsaicin, captopril, capuride, caracemide, carbachol, carbadox, carbamazepine, carbamide peroxide, carbantel lauryl sulfate, carbaspirin calcium, carbazeran, carbazomycin C, carbenicillin potassium, carbenoxolone sodium, carbetimer, carbetocin, carbidopa, carbidopa-levodopa, carbinoxamine maleate, carbiphene hydrochloride, carbocloral, carbocysteine, carbol-fuchsin, carboplatin, carboprost, carbovir, carboxamide-amino-triazole, carboxyamidotriazole, carboxymethylated beta-1,3-glucan, carbuterol hydrochloride, CaRest M3, carfentanil citrate, carisoprodol, carmantadine, carmustine, CARN 700, camidazole, caroxazone, carperitide, carphenazine maleate, carprofen, carsatrin succinate, cartazolate, carteolol, carteolol hydrochloride, cartilage derived inhibitor, carubicin hydrochloride, carumonam sodium, carvedilol, carvotroline, carvotroline hydrochloride, carzelesin, casein kinase inhibitors (ICOS), castanospermine, caurumonam, cebaracetam, cecropin B, cedefingol, cefaclor, cefadroxil, cefamandole, cefaparole, cefatrizine, cefazaflur sodium, cefazolin, cefbuperazone, cefcapene pivoxil, cefdaloxime pentexil tosilate, cefdinir, cefditoren pivoxil, cefepime, cefetamet, cefetecol, cefixime, cefluprenam, cefinenoxime hydrochloride, cefinetazole, cefminlox, cefodizime, cefonicid sodium, cefoperazone sodium, ceforamide, cefoselis, cefotaxime sodium, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftibuten, ceftizoxime sodium, ceftriaxone, cefuroxime, celastrol, celiprolol, cepacidiine A, cephacetrile sodium, cephalexin, cephaloglycin, cephaloridine, cephalothin sodium, cephapirin sodium, cephradine, cericlamine, cerivastatin, ceronapril, certoparin sodium, ceruletide, cetaben sodium, cetalkonium chloride, cetamolol hydrochloride, cetiedil, cetirizine, cetophenicol, cetraxate hydrochloride, cetrorelix, cetuximab (commercially available as ERBITUX® from Eli Lilly and Company), cetylpyridinium chloride, chenodiol, chlophedianol hydrochloride, chloral betaine, chlorambucil, chloramphenicol, chlordantoin, chlordiazepoxide, chlorhexidine gluconate, chlorins, chlormadinone acetate, chloroorienticin A, chloroprocaine hydrochloride, chloropropamide, chloroquine, chloroquinoxaline sulfonamide, chlorothiazide, chlorotrianisene, chloroxine, chloroxylenol, chlorphenesin carbamate, chlorpheniramine maleate, chlorpromazine, chlorpropamide, chlorprothixene, chlortetracycline bisulfate, chlorthalidone, chlorzoxazone, cholestyramine resin, chromonar hydrochloride, cibenzoline, cicaprost, ciclafrine hydrochloride, ciclazindol, ciclesonide, cicletanine, ciclopirox, cicloprofen, cicloprolol, cidofovir, cidoxepin hydrochloride, cifenline, ciglitazone, ciladopa hydrochloride, cilansetron, cilastatin sodium, cilazapril, cilnidipine, cilobamine mesylate, cilobradine, cilofungin, cilostazol, cimaterol, cimetidine, cimetropium bromide, cinalukast, cinanserin hydrochloride, cinepazet maleate, cinflumide, cingestol, cinitapride, cinnamedrine, cinnarizine, cinolazepam, cinoxacin, cinperene, cinromide, cintazone, cintriamide, cioteronel, cipamfylline, ciprefadol succinate, ciprocinonide, ciprofibrate, ciprofloxacin, ciprostene, ciramadol, cirolemycin, cisapride, cisatracurium besilate, cisconazole, cisplatin, cis-porphyrin, cistinexine, citalopram, citenamide, citicoline, citreamicin alpha, cladribine, clamoxyquin hydrochloride, clarithromycin, clausenamide, clavulanate potassium, clazolam, clazolimine, clebopride, clemastine, Clentiazem maleate, clidinium bromide, clinafloxacin, clindamycin, clioquinol, clioxanide, cliprofen, clobazam, clobetasol propionate, clobetasone butyrate, clocortolone acetate, clodanolene, clodazon hydrochloride, clodronic acid, clof azimine, clofibrate, clofilium phosphate, clogestone acetate, clomacran phosphate, clomegestone acetate, clometherone, clomethiazole, clomifene analogues, clominorex, clomiphene, clomipramine hydrochloride, clonazepam, clonidine, clonitrate, clonixeril, clonixin, clopamide, clopenthixol, cloperidone hydrochloride, clopidogrel (commercially available as PLAVIX® from Bristol-Myers Squibb and Sanofi Pharmaceuticals), clopimozide, clopipazan mesylate, clopirac, cloprednol, cloprostenol sodium, clorazepate dipotassium, clorethate, clorexolone, cloroperone hydrochloride, clorprenaline hydrochloride, clorsulon, clortermine hydrochloride, closantel, closiramine aceturate, clothiapine, clothixamide maleate cloticasone propionate, clotrimazole, cloxacillin benzathine, cloxyquin, clozapine, cocaine, coccidioidin, codeine, codoxime, colchicine, colestimide, colestipol hydrochloride, colestolone, colforsin, colfosceril palmitate, colistimethate sodium, colistin sulfate, collismycin A, collismycin B, colterol mesylate, combretastatin A4, combretastatin analogue, complestatin, conagenin, conorphone hydrochloride, contignasterol, contortrostatin, cormethasone acetate, corticorelin ovine triflutate, corticotropin, cortisone acetate, cortivazol, cortodoxone, cosalane, costatolide, cosyntropin, cotinine, warfarin (commercially available as COUMADIN® from Bristol-Myers Squibb), coumermycin, crambescidin 816, crilvastatin, crisnatol, cromitrile sodium, cromolyn sodium, crotamiton, cryptophycin 8, cucumariosid, cuprimyxin, curacin A, curdlan sulfate, zinc hyaluran (commercially available as CURIOSIN® from Gedeon Richter), cyclacillin, cyclazocine, cyclazosin, cyclic HPMPC, cyclindole, cycliramine maleate, cyclizine, cyclobendazole, cyclobenzaprine, cyclobut A, cyclobut G, cyclocapron, cycloguanil pamoate, cycloheximide, cyclopentanthraquinones, cyclopenthiazide, cyclopentolate hydrochloride, cyclophenazine hydrochloride, cyclophosphamide, cycloplatam, cyclopropane, cycloserine, cyclosin, cyclosporine, cyclothialidine, cyclothiazide, cyclothiazomycin, cyheptamide, cypemycin, cypenamine hydrochloride, cyprazepam, cyproheptadine hydrochloride, cyprolidol hydrochloride, cyproterone, cyproximide, cysteamine, cysteine hydrochloride, cystine, cytarabine, cytarabine hydrochloride, cytarabine ocfosfate, cytochalasin B, cytolytic factor, cytostatin, dacarbazine, dacliximab, dactimicin, dactinomycin, daidzein, daledalin tosylate, dalfopristin, dalteparin sodium, daltroban, dalvastatin, danaparoid, danazol, dantrolene, daphlnodorin A, dapiprazole, dapitant, dapoxetine hydrochloride, dapsone, daptomycin, darglitazone sodium, darifenacin, darlucin A, darodipine, darsidomine, darusentan, daunorubicin hydrochloride, dazadrol maleate, dazepinil hydrochloride, dazmegrel, dazopride fumarate, dazoxiben hydrochloride, debrisoquin sulfate, decitabine, deferiprone, deflazacort, dehydrocholic acid, dehydrodidemnin B, dehydroepiandrosterone, delapril, delapril hydrochloride, delavirdine mesylate, delequamine, delfaprazine, delmadinone acetate, delmopinol, delphinidin, demecarium bromide, demeclocycline, demecycline, demoxepam, denofungin, deoxypyridinoline, 2-propylpentanoic acid (commercially available as DEPAKOTE® from Abbott), deprodone, deprostil, depsidomycin, deramciclane, dermatan sulfate, desciclovir, descinolone acetonide, desflurane, desipramine hydrochloride, desirudin, deslanoside, deslorelin, desmopressin, desogestrel, desonide, desoximetasone, desoxoamiodarone, desoxycorticosterone acetate, detajmium bitartrate, deterenol hydrochloride, detirelix acetate, devazepide, dexamethasone, dexamisole, dexbrompheniramine maleate, dexchlorpheniramine maleate, dexclamol hydrochloride, dexetimide, dexfenfluramine hydrochloride, dexifosfamide, deximafen, dexivacaine, dexketoprofen, dexloxiglumide, dexmedetomidine, dexormaplatin, dexoxadrol hydrochloride, dexpanthenol, dexpemedolac, dexpropranolol hydrochloride, dexrazoxane, dexsotalol, dextrin 2-sulphate, dextroamphetamine, dextromethorphan, dextrorphan hydrochloride, dextrothyroxine sodium, dexverapamil, dezaguanine, dezinamide, dezocine, diacetolol hydrochloride, diamocaine cyclamate, diapamide, diatrizoate meglumine, diatrizoic acid, diaveridine, diazepam, diaziquone, diazoxide, dibenzepin hydrochloride, dibenzothiophene, dibucaine, dichliorvos, dichloralphenazone, dichlorphenamide, dicirenone, diclofenac sodium, dicloxacillin, dicranin, dicumarol, dicyclomine hydrochloride, didanosine, didemnin B, didox, dienestrol, dienogest, diethylcarbamazine citrate, diethylhomospermine, diethylnorspermine, diethylpropion hydrochloride, diethylstilbestrol, difenoximide hydrochloride, difenoxin, diflorasone diacetate, difloxacin hydrochloride, difluanine hydrochloride, diflucortolone, diflumidone sodium, diflunisal, difluprednate, diftalone, digitalis, digitoxin, digoxin, dihexyverine hydrochloride, dihydrexidine, dihydro-5-azacytidine, dihydrocodeine bitartrate, dihydroergotamine mesylate, hihydroestosterone, dihydrostreptomycin sulfate, dihydrotachysterol, dihydrotaxol, phenytoin (commercially available as DILANTIN® from Parke, Davis & Company), dilevalol hydrochloride, diltiazem hydrochloride, dimefadane, dimefline hydrochloride, dimenhydrinate, dimercaprol, dimethadione, dimethindene maleate, dimethisterone, dimethyl prostaglandin A1, dimethyl sulfoxide, dimethylhomospermine, dimiracetam, dimoxamine hydrochloride, dinoprost, dinoprostone, dioxadrol hydrochloride, dioxamycin, diphenhydramine citrate, diphenidol, diphenoxylate hydrochloride, diphenyl spiromustine, dipivefin hydrochloride, dipivefrin, dipliencyprone, diprafenone, dipropylnorspermine, dipyridamole, dipyrithione, dipyrone, dirithromycin, discodermolide, disobutamide, disofenin, disopyramide, disoxaril, disulfuram, ditekiren, divalproex sodium, dizocilpine maleate, dobutamine, docarpamine, docebenone, docetaxel, doconazole, docosanol, dofetilide, dolasetron, drotrecogin alfa (commercially available as XIGRIS® from Eli Lilly and Company), duloxetine hydrochloride (commercially available as CYMBALTA® from Eli Lilly and Company), ebastine, ebiratide, ebrotidine, ebselen, ecabapide, ecabet, ecadotril, ecdisteron, echicetin, echistatin, echothiophate iodide, eclanamine maleate, eclazolast, ecomustine, econazole, ecteinascidin 722, edaravone, edatrexate, edelfosine, edifolone acetate, edobacomab, edoxudine, edrecolomab, edrophonium chloride, edroxyprogesteone acetate, efegatran, eflornithine, efonidipine, egualcen, elantrine, eleatonin, elemene, eletriptan, elgodipine, eliprodil, elsamitrucin, eltenae, elucaine, emalkalim, emedastine, emetine hydrochloride, emiglitate, emilium tosylate, emitefur, emoctakin, enadoline hydrochloride, enalapril, enalaprilat, enalkiren, enazadrem, encyprate, endralazine mesylate, endrysone, enflurane, englitazone, enilconazole, enisoprost, enlimomab, enloplatin, enofelast, enolicam sodium, enoxacin, enoxacin, enoxaparin sodium, enoxaparin sodium, enoximone, enpiroline phosphate, enprofylline, enpromate, entacapone, enterostatin, enviradene, enviroxime, ephedrine, epicillin, epimestrol, epinephrine, epinephryl borate, epipropidine, epirizole, epirubicin, epitetracycline hydrochloride, epithiazide, epoetin alfa, epoetin beta, epoprostenol, epoprostenol sodium, epoxymexrenone, epristeride, eprosartan, eptastigmine, equilenin, equilin, erbulozole, erdosteine, ergoloid mesylates, ergonovine maleate, ergotamine tartrate, ersentilide, ersofermin, erythritol, erythrityl tetranitrate, erythromycin, esmolol hydrochloride, esomeprazole (commercially available as NEXIUM® from AstraZeneca), esorubicin hydrochloride, esproquin hydrochloride, estazolam, estradiol, estramustine, estramustine analogue, estrazinol hydrobromide, estriol, estrofurate, estrogen agonists, estrogen antagonists, estrogens, conjugated estrogens, esterified, estrone, estropipate, esuprone, etafedrine hydrochloride, etanidazole, etanterol, etarotene, etazolate hydrochloride, eterobarb, ethacizin, ethacrynate sodium, ethacrynic acid, ethambutol hydrochloride, ethamivan, ethanolamine oleate, ethehlorvynol, ether, ethinyl estradiol, ethiodized oil, ethionamide, ethonam nitrate, ethopropazine hydrochloride, ethosuximide, ethotoin, ethoxazene hydrochloride, ethybenztropine, ethyl chloride, ethyl dibunate, ethylestrenol, ethyndiol, ethynerone, ethynodiol diacetate, etibendazole, etidocaine, etidronate disodium, etidronic acid, etifenin, etintidine hydrochloride, etizolam, etodolac, etofenamate, etoformin hydrochloride, etomidate, etonogestrel, etoperidone hydrochloride, etoposide, etoprine, etoxadrol hydrochloride, etozolin, etrabamine, etretinate, etryptamine acetate, eucatropine hydrochloride, eugenol, euprocin hydrochloride, eveminomicin, exametazine, examorelin, exaprolol hydrochloride, exemestane, exetimibe (commercially available as ZETIA® from Merck), fadrozole, faeriefungin, famciclovir, famotidine (commercially available as PEPCID® from Merck), fampridine, fantof arone, fantridone hydrochloride, faropenem, fasidotril, fasudil, fazarabine, fedotozine, felbamate, felbinac, felodipine, felypressin, fenalamide, fenamole, fenbendazole, fenbufen, fencibutirol, fenclofenac, fenclonine, fenclorac, fendosal, fenestrel, fenethylline hydrochloride, fenfluramine hydrochloride, fengabine, fenimide, fenisorex, fenmetozole hydrochloride, fenmetramide, fenobam, fenoctimine sulfate, fenofibrate, fenoldopam, fenoprofen, fenoterol, fenpipalone, fenprinast hydrochloride, fenprostalene, fenquizone, fenretinide, fenspiride, fentanyl citrate, fentiazac, fenticlor, fenticonazole, fenyripol hydrochloride, fepradinol, ferpifosate sodium, ferristene, ferrixan, ferrous sulfate, ferumoxides, ferumoxsil, fetoxylate hydrochloride, fexofenadine, fezolamine fumarate, fiacitabine, fialuridine, fibrinogen I 125, filgrastim, filipin, finasteride (commercially available as PROPECIA® from Merck), flavodilol maleate, flavopiridol, flavoxate hydrochloride, flazalone, flecainide, flerobuterol, fleroxacin, flesinoxan, flestolol sulfate, fletazepam, flezelastine, flobufen, floctafenine, flomoxef, flordipine, florfenicol, florifenine, flosatidil, flosequinan, floxacillin, floxuridine, fluasterone, fluazacort, flubanilate hydrochloride, flubendazole, flucindole, flucloronide, fluconazole, flucytosine, fludalanine, fludarabine phosphate, fludazonium chloride, fludeoxyglucose F 18, fludorex, fludrocortisone acetate, flufenamic acid, flufenisal, flumazenil, flumecinol, flumequine, flumeridone, flumethasone, flumetramide, flumezapine, fluminorex, flumizole, flumoxonide, flunarizine, flunidazole, flunisolide, flunitrazepam, flunixin, fluocalcitriol, fluocinolone acetonide, fluocinonide, fluocortin butyl, fluocortolone, fluorescein, fluorodaunorunicin hydrochloride, fluorodopa F 18, fluoroformylone, fluoroquinolones, fluorometholone, fluorouracil, fluotracen hydrochloride, fluoxetine, fluoxymesterone, fluparoxan, fluperamide, fluperolone acetate, fluphenazine decanoate, flupirtine, fluprednisolone, fluproquazone, fluprostenol sodium, fluquazone, fluradoline hydrochloride, flurandrenolide, flurazepam hydrochloride, flurbiprofen, fluretofen, flurithromycin, fluorocitabine, fluorof amide, fluorogestone acetate, flurothyl, fluoroxene, fluspiperone, fluspirilene, fluticasone propionate (commercially available as ADVAIR® from GlaxoSmithKline), fluticasone furoate, flutrimazole, flutroline, fluvastatin, fluvastatin sodium, fluvoxamine, fluzinamide, folic acid, follicle regulatory protein, folliculostatin, fomepizole, fonazine mesylate, forasartan, forfenimex, forfenirmex, formestane, formocortal, formoterol, fosarilate, fosazepam, foscarnet sodium, fosfomycin, fosfonet sodium, fosinopril, fosinoprilat, fosphenyloin, fosquidone, fostedil, fostriecin, fotemustine, fuchsin, basic, fumoxicillin, fungimycin, furaprofen, furazolidone, furazolium chloride, furegrelate sodium, furobufen, furodazole, furosemide, fusidate sodium, fusidic acid, gabapentin, gadobenate dimeglumine, gadobenic acid, gadobutrol, gadodiamide, gadolinium texaphyrin, gadopentetate dimegiumine, gadoteric acid, gadoteridol, gadoversetamide, galantamine, galdansetron, galdansetron hydrochloride, gallamine triethiodide, gallium nitrate, gallopamil, galocitabine, gamfexine, gamolenic acid, ganciclovir, ganirelix, ganirelix acetate, gelatinase inhibitors, gemcadiol, gemcitabine (commercially available as GEMZAR® from Eli Lilly and Company), gemeprost, gemfibrozil, gentamicin sulfate, gentian violet, gepirone, gestaclone, gestodene, gestonorone caproate, gestrinone, gevotroline hydrochloride, girisopam, glaspimod, glaucocalyxin A, glemanserin, gliamilide, glibornuride, glicetanile sodium, gliflumide, glimepiride, glipizide, gloximonam, glucagon, glutapyrone, glutathione inhibitors, glutethimide, glyburide, glycopine, glycopril, glycopyrrolate, glyhexamide, glymidine sodium, glyoctamide, glyparamide, colloidal gold Au 198, gonadoctrinins, gonadorelin, gonadotropins, goserelin, gramicidin, granisetron, grepafloxacin, griseofulvin, guaiapate, guaithylline, guanabenz, guanabenz acetate, guanadrel sulfate, guancydine, guanethidine monosulfate, guanfacine hydrochloride, guanisoquin sulfate, guanoclor sulfate, guanoctine hydrochloride, guanoxabenz, guanoxan sulfate, guanoxyfen sulfate, gusperimus trihydrochloride, halazepam, halcinonide, halichondrin B, halobetasol propionate, halof antrine, halof antrine hydrochloride, halofenate, halofuginone hydrobromide, halomon, galopemide, galoperidol, halopredone, haloprogesterone, haloprogin, halothane, halquinols, hamycin, han menopausal gonadotropins, hatomamicin, hatomarubigin A, hatomarubigin B, hatomarubigin C, hatomarubigin D, heparin sodium, hepsulfam, heregulin, hetacillin, heteronium bromide, hexachlorophene:hydrogen peroxide, hexafluorenium bromide, hexamethylene bisacetamide, hexedine, hexobendine, hexoprenaline sulfate, hexylresorcinol, histamine phosphate, histidine, histoplasmin, histrelin, homatropine hydrobromide, hoquizil hydrochloride, human chorionic gonadotropin, hycanthone, hydralazine hydrochloride, hydralazine polistirex, hydrochlorothiazide, hydrocodone bitartrate, hydrocortisone, hydroflumethiazide, hydromorphone hydrochloride, hydroxyamphetamine hydrobromide, hydroxychloroquine sulfate, hydroxyphenamate, hydroxyprogesterone caproate, hydroxyurca, hydroxyzine hydrochloride, hymecromone, hyoscyamine, hypericin, ibafloxacin, ibandronic acid, ibogaine, ibopamine, ibudilast, ibufenac, ibuprofen, ibutilide fumarate, icatibant acetate, ichthammol, icotidine, idarubicin, idoxifene, idoxuridine, idramantone, iemefloxacin, iesopitron, ifetroban, ifosfamide, ilepeimide, illimaquinone, ilmofosine, ilomastat, ilonidap, iloperidone, iloprost, imafen hydrochloride, imazodan hydrochloride, imidapril, imidazoacridones, imidecyl iodine, imidocarb hydrochloride, imidoline hydrochloride, imidurea, imiloxan hydrochloride, imipenem, imipramine hydrochloride, imiquimod, immunostimulant peptides, impromidine hydrochloride, indacrinone, indapamide, indecamide hydrochloride, indeloxazine hydrochloride, indigotindisulfonate sodium, indinavir, indocyanine green, indolapril hydrochloride, indolidan, indometacin, indomethacin sodium, indoprofen, indoramin, indorenate hydrochloride, indoxole, indriline hydrochloride, infliximab (commercially available as REMICADE® from Janssen Biotech, Inc.), inocoterone, inogatran, inolimomab, inositol niacinate, insulin, insulin glargine (commercially available as LANTUS® from Sanofi-Aventis), interferons, interferon beta-1a (commercially available as AVONEX® from BIOGEN), interleukins, intrazole, intriptyline hydrochloride, iobenguane, iobenzamic acid, iobitridol, iocarmate meglumine, iocarmic acid, iocetamic acid, iodamide, iodine, iodipamide meglumine, iodixanol, iodoamiloride, iodoantipyrine I 131, iodocholesterol I 131, iododoxorubicin, iodohippurate sodium I 131, iodopyracet I 125, iodoquinol, iodoxamate meglumine, iodoxamie acid, ioglicic acid, iofetamine hydrochloride I 123, iofratol, ioglucol, ioglucomide, ioglycamic acid, iogulamide, iohexyl, iomeprol, iomethin I 125, iopamidol, iopanoic acid, iopentol, iophendylate, ioprocemic acid, iopromide, iopronic acid, iopydol, iopydone, iopyrol, iosefamic acid, ioseric acid, iosulamide meglumine, iosumetic acid, iotasul, iotetric acid, iothalamate sodium, iothalamic acid, iotriside, iotrolan, iotroxic acid, iotyrosine I 131, ioversol, ioxagiate sodium, ioxaglate meglumine, ioxaglic acid, ioxilan, ioxotrizoic acid, ipazilide, ipenoxazone, ipidacrine, ipodate calcium, ipomeanol, 4-, ipratropium bromide, ipriflavone, iprindole, iprofenin, ipronidazole, iproplatin, iproxamine hydrochloride, ipsapirone, irbesartan, irinotecan, irloxacin, iroplact, irsogladine, irtemazole, isalsteine, isamoxole, isbogrel, isepamicin, isobengazole, isobutamben, isocarboxazid, isoconazole, isoetharine, isofloxythepin, isoflupredone acetate, isoflurane, isofluorophate, isohomohalicondrin B, isoleucine, isomazole hydrochloride, isomylamine hydrochloride, isoniazid, isopropamide iodide, isopropyl alcohol, isopropyl unoprostone, isoproterenol hydrochloride, isosorbide, isosorbide mononitrate, isotiquimide, isotretinoin, isoxepac, isoxicam, isoxsuprine hydrochloride, isradipine, itameline, itasetron, itazigrel, itopride, itraconazole, ivermectin, jasplakinolide, josamycin, kahalalide F, kalafungin, kanamycin sulfate, ketamine hydrochloride, ketanserin, ketazocine, ketazolam, kethoxal, ketipramine fumarate, ketoconazole, ketoprofen, ketorfanol, ketorolac, ketotifen fumarate, kitasamycin, labetalol hydrochloride, lacidipine, lacidipine, lactitol, lactivicin, laennec, lafutidine, lamellarin-n triacetate, lamifiban, lamivudine, lamotrigine, lanoconazole, LANOXIN® (digoxin, available from GlaxoSmithKline), lanperisone, lanreotide, lansoprazole (commercially available as PREVAID® from Takeda Pharmaceuticals, Inc.), latanoprost, lateritin, laurocapram, lauryl isoquinolinium bromide, lavoltidine succinate, lazabemide, lecimibide, leinamycin, lemildipine, leminoprazole, lenercept, leniquinsin, lenograstim, lenperone, lentinan sulfate, leptin, leptolstatin, lercanidipine, lergotrile, lerisetron, letimide hydrochloride, letrazuril, letrozole, leucine, leucomyzin, leuprolide acetate, leuprolide, leuprorelin, levamfetamine succinate, levamisole, levdobutamine lactobionate, levcromakalim, levetiracetam, levobetaxolol, levobunolol, levobupivacaine, levocabastine, levocarnitine, levodopa, levodropropizine, levofloxacin (commercially available as LEVAQUIN® from Jessen Pharmaceuticals, Inc.), levofuraltadone, levoleucovorin calcium, levomethadyl acetate, levomethadyl acetate hydrochloride, levomoprolol, levonantradol hydrochloride, levonordefrin, levonorgestrel, levopropoxyphene napsylate, levopropylcillin potassium, levormeloxifene, levorphanol tartrate, levosimendan, levosulpiride, levothyroxine sodium, levoxadrol hydrochloride, lexipafant, lexithromycin, liarozole, libenzapril, lidamidine hydrochloride, lidocaine, lidofenin, lidoflazine, lifarizine, lifibrate, lifibrol, linarotene, lincomycin, linear polyamine analogue, linogliride, linopirdine, linotroban, linsidomine, lintitript, lintopride, liothyronine I 125, liothyronine sodium, liotrix, lirexapride, lisinopril, lissoclinamide 7, lixazinone sulfate, lobaplatin, lobenzarit sodium, lobucavir, lodelaben, lodoxamide, lofemizole hydrochloride, lofentanil oxalate, lofepramine hydrochloride, lofexidine hydrochloride, lombricine, lomefloxacin, lomerizine, lometraline hydrochloride, lometrexol, lomitapide, lomofungin, lomoxicam, lomustine, lonapalene, lonazolac, lonidamine, loperamide hydrochloride, loracarbef, lorajmine hydrochloride, loratadine, lorazepam, lorbamate, lorcamide hydrochloride, loreclezole, lorglumide, lormetazepam, lornoxicam, lornoxicam, lortalamine, lorzafone, losartan (commercially available as COZAAR® from Merck), losigamone, losoxantrone, losulazine hydrochloride, loteprednol, lovastatin, loviride, loxapine, loxoribine, lubeluzole, lucanthone hydrochloride, lufironil, lurosetron mesylate, lurtotecan, luteinizing hormone, lutetium, lutrelin acetate, luzindole, lyapolate sodium, lycetamine, lydicamycin, lydimycin, lynestrenol, lypressin, lysine, lysofylline, lysostaphin, lytic peptides, maduramicin, mafenide, magainin 2 amide, magnesium salicylate, magnesium sulfate, magnolol, maitansine, malethamer, mallotochromene, mallotojaponin, malotilate, mangafodipir, manidipine, maniwamycin A, mannitol, mannostatin A, manumycin E, manumycin F, MAPK/ERK kinase (MEK) inhibitors, mapinastine, maprotiline, marimastat, masoprocol, maspin, massetolide, matrilysin inhibitors, maytansine, mazapertine succiniate, mazindol, mebendazole, mebeverine hydrochloride, mebrofenin, mebutamate, mecamylamine hydrochloride, mechlorethamine hydrochloride, meclocycline, meclofenamate sodium, mecloqualone, meclorisone dibutyrate, medazepam hydrochloride, medorinone, medrogestone, medroxalol, medroxyprogesterone (commercially available as DEPO-PROVERA® from Pfizer, Inc.), medrysone, meelizine hydrochloride, mefenamic acid, mefenidil, mefenorex hydrochloride, mefexamide, mefloquine hydrochloride, mefruside, megalomicin potassium phosphate, megestrol acetate, meglumine, meglutol, melengestrol acetate, melitracen hydrochloride, melphalan, memotine hydrochloride, menabitan hydrochloride, menoctone, menogaril, menotropins, meobentine sulfate, mepartricin, mepenzolate bromide, meperidine hydrochloride, mephentermine sulfate, mephenyloin, mephobarbital, mepivacaine hydrochloride, meprobamate, meptazinol hydrochloride, mequidox, meralein sodium, merbarone, mercaptopurine, mercufenol chloride, mercury, meropenem, mesalamine, meseclazone, mesoridazine, mesterolone, mestranol, mesuprine hydrochloride, metalol hydrochloride, metaproterenol polistirex, metaraminol bitartrate, metaxalone, meteneprost, meterelin, metformin, methacholine chloride, methacycline, methadone hydrochloride, methadyl acetate, methalthiazide, methamphetamine hydrochloride, methaqualone, methazolamide, methdilazine, methenamine, methenolone acetate, methetoin, methicillin sodium, methimazole, methioninase, methionine, methisazone, methixene hydrochloride, methocarbamol, methohexital sodium, methopholine, methotrexate, methotrimeprazine, methoxatone, methoxyflurane, methsuximide, methyclothiazide, methyl 10 palmoxirate, methylatropine nitrate, methylbenzethonium chloride, methyldopa, methyldopate hydrochloride, methylene blue, methylergonovine maleate, methylhistamine, R-alpha, methylinosine monophosphate, methylphenidate hydrochloride, methylprednisolone, methyltestosterone, methynodiol diacelate, methysergide, methysergide maleate, metiamide, metiapine, metioprim, metipamide, metipranolol, metizoline hydrochloride, metkephamid acetate, metoclopramide, metocurine iodide, metogest, metolazone, metopimazine, metoprine, metoprolol, metoquizine, metrifonate, metrizamide, metrizoate sodium, metronidazole, meturedepa, metyrapone, metyrosine, mexiletine hydrochloride, mexrenoate potassium, mezlocillin, mfonelic acid, mianserin hydrochloride, mibefradil, mibefradil dihydrochloride, mibolerone, michellamine B, miconazole, microcolin A, midaflur, midazolam hydrochloride, midodrine, mifepristone, mifobate, miglitol, milacemide, milameline, mildronate, milenperone, milipertine, milnacipran, milrinone, miltefosine, mimbane hydrochloride, minaprine, minaxolone, minocromil, minocycline, minoxidil, mioflazine hydrochloride, miokamycin, mipragoside, mirfentanil, mirimostim, mirincamycin hydrochloride, mirisetron maleate, mirtazapine, mismatched double stranded RNA, misonidazole, misoprostol, mitindomide, mitocarcin, mitocromin, mitogillin, mitoguazone, mitolactol, mitomalcin, mitomycin, mitonafide, mitosper, mitotane, mitoxantrone, mivacurium chloride, mivazerol, mixanpril, mixidine, mizolastine, mizoribine, moclobemide, modafinil, modaline sulfate, modecamide, moexipril, mof arotene, mofegiline hydrochloride, mofezolac, molgramostim, molinazone, molindone hydrochloride, molsidomine, mometasone, monatepil maleate, monensin, monoctanoin, montelukast sodium (commercially available as SINGULAIR® available from Merck), montirelin, mopidamol, moracizine, morantel tartrate, moricizine, morniflumate, morphine, morphine sulfate, morrhuate sodium, mosapramine, mosapride, motilide, motretinide, moxalactam disodium, moxazocine, moxiraprine, moxnidazole, moxonidine, mumps skin test antigen, mustard anticancer agent, muzolimine, mycaperoxide B, mycophenolic acid, myriaporone, nabazenil, nabilone, nabitan hydrochloride, naboctate hydrochloride, nabumetone, n-acetyldinaline, nadide, nadifloxacin, nadolol, nadroparin calcium, nafadotride, nafamostat, nafarelin, nafcillin sodium, nafenopin, nafimidone hydrochloride, naflocort, nafomine malate, nafoxidine hydrochloride, nafronyl oxalate, naftifine hydrochloride, naftopidil, naglivan, nagrestip, nalbuphine hydrochloride, nalidixate sodium, nalidixic acid, nalmefene, nalmexone hydrochloride, naloxone/pentazocine, naltrexone, namoxyrate, nandrolone phenpropionate, nantradol hydrochloride, napactadine hydrochloride, napadisilate, napamezole hydrochloride, napaviin, naphazoline hydrochloride, naphterpin, naproxen, naproxol, napsagatran, naranol hydrochloride, narasin, naratriptan, nartograstim, nasaruplase, natamycin, nateplase, naxagolide hydrochloride, nebivolol, nebramycin, nedaplatin, nedocromil, nefazodone hydrochloride, neflumozide hydrochloride, nefopam hydrochloride, nelezaprine maleate, nemazoline hydrochloride, nemorubicin, neomycin palmitate, neostigmine bromide, neridronic acid, netilmicin sulfate, neutral endopeptidase, neutramycin, nevirapine, nexeridine hydrochloride, niacin, nibroxane, nicardipine hydrochloride, nicergoline, niclosamide, nicorandil, nicotinyl alcohol, nicotine (commercially available as NICOTROL® NS from Pfizer, Inc.), nifedipine, nifirmerone, nifluridide, nifuradene, nifuraldezone, nifuratel, nifuratrone, nifurdazil, nifurimide, nifurpirinol, nifurquinazol, nifurthiazole, nilutamide, nilvadipine, nimazone, nimodipine, niperotidine, niravoline, niridazole, nisamycin, nisbuterol mesylate, nisin, nisobamate, nisoldipine, nisoxetine, nisterime acetate, nitarsone, nitazoxamide, nitecapone, nitrafudam hydrochloride, nitralamine hydrochloride, nitramisole hydrochloride, nitrazepam, nitrendipine, nitrocycline, nitrodan, nitrofurantoin, nitrofurazone, nitroglycerin, nitromersol, nitromide, nitromifene citrate, nitrous oxide, nitroxide antioxidant, nitrullyn, nivazol, nivimedone sodium, nizatidine, noberastine, nocodazole, nogalamycin, nolinium bromide, nomifensine maleate, noracymethadol hydrochloride, norbolethone, norepinephrine bitartrate, norethindrone, norethynodrel, norfloxacin, norflurane, norgestimate, norgestomet, norgestrel, nortriptyline hydrochloride, noscapine, novobiocin sodium, N-substituted benzaimides, nufenoxole, nylestriol, nystatin, O6-benzylguanine, obidoxime chloride, ocaperidone, ocfentanil hydrochloride, ocinaplon, octanoic acid, octazamide, octenidine hydrochloride, octodrine, octreotide, octriptyline phosphate, ofloxacin, oformine, okicenone, olanzapine (commercially available as ZYPREXA® from Eli Lilly and Company), oligonucleotides, olopatadine, olprinone, olsalazine, olsalazine sodium, olvanil, omeprazole, onapristone, ondansetron, ontazolast, oocyte maturation inhibitor, opipramol hydrochloride, oracin, orconazole nitrate, orgotein, orlislat, ormaplatin, ormetoprim, ornidazole, orpanoxin, orphenadrine citrate, osaterone, otenzepad, oxacillin sodium, oxagrelate, oxaliplatin, oxamarin hydrochloride, oxamisole, oxamniquine, oxandrolone, oxantel pamoate, oxaprotiline hydrochloride, oxaprozin, oxarbazole, oxatomide, oxaunomycin, oxazepam, oxcarbazepine, oxendolone, oxethazaine, oxetorone fumarate, oxfendazole, oxfenicine, oxibendazole, oxiconazole, oxidopamine, oxidronic acid, oxifungin hydrochloride, oxilorphan, oximonam, oximonam sodium, oxiperomide, oxiracetam, oxiramide, oxisuran, oxmetidine hydrochloride, oxodipine, oxogestone phenpropionate, oxolinic acid, oxprenolol hydrochloride, oxtriphylline, oxybutynin chloride, oxychlorosene, oxycodone, oxymetazoline hydrochloride, oxymetholone, oxymorphone hydrochloride, oxypertine, oxyphenbutazone, oxypurinol, oxytetracycline, oxytocin, ozagrel, ozolinone, paclitaxel, palauamine, paldimycin, palinavir, paliperidone (commercially available as INVEGA® from Janssen Pharmaceuticals, Inc.), paliperidone palmitate (commercially available as INVEGA® SUSTENNA® from Janssen Pharmaceuticals, Inc.), palmitoylrhizoxin, palmoxirate sodium, pamaqueside, pamatolol sulfate, pamicogrel, pamidronate disodium, pamidronic acid, panadiplon, panamesine, panaxytriol, pancopride, pancuronium bromide, panipenem, pannorin, panomifene, pantethine, pantoprazole, papaverine hydrochloride, parabactin, parachlorophenol, paraldehyde, paramethasone acetate, paranyline hydrochloride, parapenzolate bromide, pararosaniline pamoate, parbendazole, parconazole hydrochloride, paregoric, pareptide sulfate, pargyline hydrochloride, parnaparin sodium, paromomycin sulfate, paroxetine (commercially available as PAXIL® from GlaxoSmithKlein), parthenolide, partricin, paulomycin, pazelliptine, pazinaclone, pazoxide, pazufloxacin, pefloxacin, pegaspargase, pegorgotein, pelanserin hydrochloride, peldesine, peliomycin, pelretin, pelrinone hydrochloride, pemedolac, pemerid nitrate, pemetrexed, pemirolast, pemoline, penamecillin, penbutolol sulfate, penciclovir, penfluridol, penicillin G benzathine, penicillin G potassium, penicillin G procaine, penicillin G Sodium, penicillin V, penicillin V benzathine, penicillin V hydrabamine, penicillin V potassium, pentabamate, pentaerythritol tetranitrate, pentafuside, pentamidine, pentamorphone, bentamustine, pentapiperium methylsulfate, pentazocine, pentetic acid, pentiapine maleate, pentigetide, pentisomicin, pentizidone sodium, pentobarbital, pentomone, pentopril, pentosan, pentostatin, pentoxifylline, pentrinitrol, pentrozole, peplomycin sulfate, pepstatin, perflubron, perfof amide, perfosfamide, pergolide, perhexyline maleate, perillyl alcohol, perindopril, perindoprilat, perlapine, permethrin, perospirone, perphenazine, phenacemide, phenaridine, phenazinomycin, phenazopyridine hydrochloride, phenbutazone sodium glycerate, phencarbamide, phencyclidine hydrochloride, phendimetrazine tartrate, phenelzine sulfate, phenmetrazine hydrochloride, phenobarbital, phenoxybenzamine hydrochloride, phenprocoumon, phenserine, phensuccinal, phensuximide, phentermine, phentermine hydrochloride, phentolamine mesilate, phentoxifylline, phenyl aminosalicylate, phenylacetate, phenylalanine, phenylalanyl ketoconazole, phenylbutazone, phenylephrine hydrochloride, phenylpropanolamine hydrochloride, phenylpropanolamine polistirex, phenyramidol hydrochloride, phenyloin, phosphatase inhibitors, physostigmine, picenadol, picibanil, picotrin diolamine, picroliv, picumeterol, pidotimod, pifamine, pilocarpine, pilsicamide, pimagedine, pimetine hydrochloride, pimilprost, pimobendan, pimozide, pinacidil, pinadoline, pindolol, pinnenol, pinocebrin, pinoxepin hydrochloride, pioglitazone (commercially available as ACTOS® from Takeda Pharmaceuticals), pipamperone, pipazethate, pipecuronium bromide, piperacetazine, piperacillin sodium, piperamide maleate, piperazine, pipobroman, piposulfan, pipotiazine palmitate, pipoxolan hydrochloride, piprozolin, piquindone hydrochloride, piquizil hydrochloride, piracetam, pirandamine hydrochloride, pirarubicin, pirazmonam sodium, pirazolac, pirbenicillin sodium, pirbuterol acetate, pirenperone, pirenzepine hydrochloride, piretanide, pirfenidone, piridicillin sodium, piridronate sodium, piriprost, piritrexim, pirlimycin hydrochloride, pirlindole, pirmagrel, pirmenol hydrochloride, pirnabine, piroctone, pirodavir, pirodomast, pirogliride tartrate, pirolate, pirolazamide, piroxantrone hydrochloride, piroxicam, piroximone, pirprofen, pirquinozol, pirsidomine, prenylamine, pitavastatin (commercially available as LIVALOA® from Eli Lilly and Company), pituitary, posterior, pivampicillin hydrochloride, pivopril, pizotyline, placetin A, platinum compounds, platinum-triamine complex, plicamycin, plomestane, pobilukast edamine, podofilox, poisonoak extract, poldine methylsulfate, poliglusam, polignate sodium, polymyxin B sulfate, polythiazide, ponalrestat, porfimer sodium, porfiromycin, potassium chloride, potassium iodide, potassium permanganate, povidone-iodine, practolol, pralidoxime chloride, pramiracetam hydrochloride, pramoxine hydrochloride, pranolium chloride, prasugrel (commercially available as EFFIENT® from Eli Lilly and Company), pravadoline maleate, pravastatin, prazepam, prazosin, prazosin hydrochloride, prednazate, prednicarbate, prednimustine, prednisolone, prednisone, prednival, pregabalin (commercially available as LYRICA® from Pfizer, Inc.), pregnenolone succiniate, prenalterol hydrochloride, pridefine hydrochloride, prifelone, prilocalne hydrochloride, prilosec, primaquine phosphate, primidolol, primidone, prinivil, prinomide tromethamine, prinoxodan, prizidilol hydrochloride, proadifen hydrochloride, probenecid, probicromil calcium, probucol, procainamide hydrochloride, procaine hydrochloride, procarbazine hydrochloride, procaterol hydrochloride, prochlorperazine, procinonide, proclonol, procyclidine hydrochloride, prodilidine hydrochloride, prodolic acid, prof adol hydrochloride, progabide, progesterone, proglumide, proinsulin human, proline, prolintane hydrochloride, promazine hydrochloride, promethazine hydrochloride, propafenone hydrochloride, propagermanium, propanidid, propantheline bromide, proparacaine hydrochloride, propatyl nitrate, propentofylline, propenzolate hydrochloride, propikacin, propiomazine, propionic acid, propionylcarnitine, propiram, propiram+paracetamol, propiverine, propofol, propoxycaine hydrochloride, propoxyphene hydrochloride, propranolol hydrochloride, propulsid, propyl bis-acridone, propylhexedrine, propyliodone, propylthiouracil, proquazone, prorenoate potassium, proroxan hydrochloride, proscillaridin, prostalene, prostratin, protamine sulfate, protegrin, protirelin, protosufloxacin, protriptyline hydrochloride, proxazole, proxazole citrate, proxicromil, proxorphan tartrate, prulifloxacin, pseudoephedrine hydrochloride, desloratadine/pseudoephedrine sulfate (commercially available as CLARINEX-D® from Merck), puromycin, purpurins, pyrabrom, pyrantel, pamoate, pyrazinamide, pyrazofurin, pyrazoloacridine, pyridostigmine bromide, pyrilamine maleate, pyrimethamine, pyrinoline, pyrithione sodium, pyrithione zinc, pyrovalerone hydrochloride, pyroxamine maleate, pyrrocaine, pyrroliphene hydrochloride, pyrroinitrin, pyrvinium pamoate, quadazocine mesylate, quazepam, quazinone, quazodine, quazolast, quetiapine (commercially available as SEROQUEL® available from AstraZenica), quiflapon, quinagolide, quinaldine blue, quinapril, quinaprilat, quinazosin hydrochloride, quinbolone, quinctolate, quindecamine acetate, quindonium bromide, quinelorane hydrochloride, quinestrol, quinfamide, quingestanol acetate, quingestrone, quinidine gluconate, quinielorane hydrochloride, quinine sulfate, quinpirole hydrochloride, quinterenol sulfate, quinuclium bromide, quinupristin, quipazine maleate, rabeprazole sodium, racephenicol, racepinephrine, raf antagonists, rafoxanide, ralitoline, raloxifene, raltitrexed, ramatroban, ramipril, ramoplanin, ramosetron, ranelic acid, ranimycin, ranitidine, ranolazine, rauwolfia serpentina, recainam, recainam hydrochloride, reclazepam, regavirumab, regramostim, relaxin, relomycin, remacemide hydrochloride, remifentanil hydrochloride, remiprostol, remoxipride, repirinast, repromicin, reproterol hydrochloride, reserpine, resinferatoxin, resorcinol, retelliptine demethylated, reticulon, reviparin sodium, revizinone, rhenium re 186 etidronate, rhizoxin, ribaminol, ribavirin, riboprine, ribozymes, ricasetron, ridogrel, rifabutin, rifametane, rifamexil, rifamide, rifampin, rifapentine, rifaximin, retinamide, rilopirox, riluzole, rimantadine, rimcazole hydrochloride, rimexolone, rimiterol hydrobromide, rimoprogin, riodipine, rioprostil, ripazepam, ripisartan, risedronate sodium, risedronic acid, risocaine, risotilide hydrochloride, rispenzepine, risperdal, risperidone, ritanserin, ritipenem, ritodrine, ritolukast, ritonavir, rizatriptan benzoate, rocastine hydrochloride, rocuronium bromide, rodocaine, roflurane, rogletimide, rohitukine, rokitamycin, roletamicide, rolgamidine, rolicyprine, rolipram, rolitetracycline, rolodine, romazarit, romurtide, ronidazole, ropinirole (commercially available as REQUIP® from GlaxoSmithKline), ropitoin hydrochloride, ropivacaine, ropizine, roquinimex, rosaramicin, rosoxacin, rotoxamine, rosuvastatin (commercially available as CRESTOR® available from AstraZeneca), roxaitidine, roxarsone, roxindole, roxithromycin, rubiginone B1, ruboxyl, rufloxacin, rupatidine, rutamycin, ruzadolane, sabeluzole, safingol, safironil, saintopin, salbutamol, salcolex, salethamide maleate, salicyl alcohol, salicylamide, salicylate meglumine, salicylic acid, salmeterol, salnacediin, salsalate, sameridine, sampatrilat, sancycline, sanfetrinem, sanguinarium chloride, saperconazole, saprisartan, sapropterin, saquinavir, sarafloxacin hydrochloride, saralasin acetate, SarCNU, sarcophytol A, sargramostim, sarmoxicillin, sarpicillin, sarpogrelate, saruplase, saterinone, satigrel, satumomab pendetide, schick test control, scopafungin, scopolamine hydrobromide, scrazaipine hydrochloride, sdi 1 mimetics, secalciferol, secobarbital, seelzone, seglitide acetate, selegiline, selegiline hydrochloride, selenium sulfide, selenomethionine se 75, selfotel, sematilide, semduramicin, semotiadil, semustine, sense oligonucleotides, sepazonium chloride, seperidol hydrochloride, seprilose, seproxetine hydrochloride, seractide acetate, sergolexole maleate, serine, sermetacin, sermorelin acetate, sertaconazole, sertindole, sertraline, setiptiline, setoperone, sevirumab, sevoflurane, sezolamide, sibopirdine, sibutramine hydrochloride, signal transduction inhibitors, silandrone, sildenafil (commercially available as VIAGRA@ from Pfizer Inc.), silipide, silteplase, silver nitrate, simendan, simtrazene, simvastatin (commercially available as ZOCOR® from Merck), sincalide, sinefungin, sinitrodil, sinnabidol, sipatrigine, sirolimus, sisomicin, sitogluside, sizofuran, sobuzoxane, sodium amylosulfate, sodium iodide I 123, sodium nitroprusside, sodium oxybate, sodium phenylacetate, sodium salicylate, solverol, solypertine tartrate, somalapor, somantadine hydrochloride, somatomedin B, somatomedin C, somatrem, somatropin, somenopor, somidobove, sonermin, sorbinil, sorivudine, sotalol, soterenol hydrochloride, sparfloxacin, sparfosate sodium, sparfosic acid, sparsomycin, sparteine sulfate, spectinomycin hydrochloride, spicamycin D, spiperone, spiradoline mesylate, spiramycin, spirapril hydrochloride, spiraprilat, spirogermanium hydrochloride, spiromustine, spironolactone, spiroplatin, spiroxasone, splenopentin, spongistatin 1, sprodiamide, squalamine, stallimycin hydrochloride, stannous pyrophosphate, stannous sulfur colloid, stanozolol, statolon, staurosporine, stavudine, steffimycin, stenbolone acetate, stepronin, stilbazium iodide, stilonium iodide, stipiamide, stiripentol, stobadine, streptomycin sulfate, streptonicozid, streptonigrin, streptozocin, stromelysin inhibitors, strontium chloride Sr 89, succibun, succimer, succinylcholine chloride, sucralfate, sucrosofate potassium, sudoxicam, sufentanil, sufotidine, sulazepam, sulbactam pivoxil, sulconazole nitrate, sulfabenz, sulfabenzamide, sulfacetamide, sulfacytine, sulfadiazine, sulfadoxine, sulfalene, sulfamerazine, sulfameter, sulfamethazine, sulfamethizole, sulfamethoxazole, sulfamonomethoxine, sulfamoxole, sulfanilate zinc, sulfanitran, sulfasalazine, sulfasomizole, sulfazamet, sulfinalol hydrochloride, sulfinosine, sulfinpyrazone, sulfisoxazole, sulfomyxin, sulfonterol hydrochloride, sulfoxamine, sulinldac, sulmarin, sulnidazole, suloctidil, sulofenur, sulopenem, suloxifen oxalate, sulpiride, sulprostone, sultamicillin, sulthiame, sultopride, sulukast, sumarotene, sumatriptan, suncillin sodium, suproclone, suprofen, suradista, suramin, surfomer, suricamide maleate, suritozole, suronacrine maleate, suxemerid sulfate, swainsonine, symakalim, symclosene, symetine hydrochloride, synthetic glycosaminoglycans, tadalafil (commercially available as CIALIS® and ACIRCA® from from Eli Lilly and Company), taciamine hydrochloride, tacrine hydrochloride, tacrolimus, talampicillin hydrochloride, taleranol, talisomycin, tallimustine, talmetacin, talniflumate, talopram hydrochloride, talosalate, tametraline hydrochloride, tamoxifen (commercially available as NOLVADEX® from AstraZeneca), tampramine fumarate, tamsulosin hydrochloride, tandamine hydrochloride, tandospirone, tapgen, taprostene, tasosartan, tauromustine, taxane, taxoid, tazadolene succinate, tazanolast, tazarotene, tazifylline hydrochloride, tazobactam, tazofelone, tazolol hydrochloride, tebufelone, tebuquine, technetium Tc 99 m bicisate, teclozan, tecogalan sodium, teecleukin, teflurane, tegafur, tegretol, teicoplanin, telenzepine, tellurapyrylium, telmesteine, telmisartan, telomerase inhibitors, teloxantrone hydrochloride, teludipine hydrochloride, temafloxacin hydrochloride, tematropium methyl sulfate, temazepam, temelastine, temocapril, temocillin, temoporfin, temozolomide, tenofovir, tenidap, teniposide, tenosal, tenoxicam, tepirindole, tepoxalin, teprotide, terazosin, terbinafine, terbutaline sulfate (commercially available as BRICANYL® from AstraZeneca), terconazole, terfenadine, terflavoxate, terguride, teriparatide acetate, terlakiren, terlipressin, terodiline, teroxalene hydrochloride, teroxirone, tertatolol, tesicam, tesimide, testolactone, testosterone, tetracaine, tetrachlorodecaoxide, tetracycline, tetrahydrozoline hydrochloride, tetramisole hydrochloride, tetrazolast meglumine, tetrazomine, tetrofosmin, tetroquinone, tetroxoprim, tetrydamine, thaliblastine, thalidomide, theofibrate, theophylline, thiabendazole, thiamiprine, thiamphenicol, thiamylal, thiazesim hydrochloride, thiazinamium chloride, thiazolidinedione, thiethylperazine, thimerfonate sodium, thimerosal, thiocoraline, thiofedrine, thioguanine, thiomarinol, thiopental sodium, thioperamide, thioridazine, thiotepa, thiothixene, thiphenamil hydrochloride, thiphencillin potassium, thiram, thozalinone, threonine, thrombin, thrombopoietin, thrombopoietin mimetic, thymalfasin, thymopoietin receptor agonist, thymotrinan, thyromedan hydrochloride, thyroxine 1 125, thyroxine 1 131, tiacrilast, tiacrilast sodium, tiagabine, tiamenidine, tianeptine, tiapafant, tiapamil hydrochloride, tiaramide hydrochloride, tiazofurin, tibenelast sodium, tibolone, tibric acid, ticabesone propionate, ticarbodine, ticarcillin cresyl sodium, ticlatone, ticlopidine, ticrynafen, tienoxolol, tifurac sodium, tigemonam dicholine, tigestol, tiletamine hydrochloride, tilidine hydrochloride, tilisolol, tilnoprofen arbamel, tilorone hydrochloride, tiludronate disodium, tiludronic acid, timefurone, timobesone acetate, timolol, tin ethyl etiopurpurin, tinabinol, timidazole, tinzaparin sodium, tioconazole, tiodazosin, tiodonium chloride, tioperidone hydrochloride, tiopinac, tiospirone hydrochloride, tiotidine, tiotropium bromide, tioxidazole, tipentosin hydrochloride, tipredane, tiprenolol hydrochloride, tiprinast meglumine, tipropidil hydrochloride, tiqueside, tiquinamide hydrochloride, tirandalydigin, tirapazamine, tirilazad, tirofiban, tiropramide, titanocene dichloride, tixanox, tixocortol pivalate, tizanidine hydrochloride, tobramycin, tocamide, tocamphyl, tofenacin hydrochloride, tolamolol, tolazamide, tolazoline hydrochloride, tolbutamide, tolcapone, tolciclate, tolfamide, tolgabide, lamotrigine, tolimidone, tolindate, tolmetin, tolnaftate, tolpovidone 1 131, tolpyrramide, tolrestat, tomelukast, tomoxetine hydrochloride, tonazocine mesylate, topiramate, topotecan, topotecan hydrochloride, topsentin, topterone, toquizine, torasemide, toremifene, torsemide, tosifen, tosufloxacin, totipotent stem cell factor, tracazolate, trafermin, tralonide, tramadol hydrochloride, tramazoline hydrochloride, trandolapril, tranexamic acid, tranilast, transcamide, translation inhibitors, trastuzumab (commercially available as HERCEPTIN® from Genentech), traxanox, trazodone hydrochloride, trazodone-hcl, trebenzomine hydrochloride, trefentanil hydrochloride, treloxinate, trepipam maleate, trestolone acetate, tretinoin, triacetin, triacetyluridine, triafungin, triamcinolone, triampyzine sulfate, triamterene, triazolam, tribenoside, tricaprilin, tricetamide, trichlormethiazide, trichohyalin, triciribine, tricitrates, triclofenol piperazine, triclofos sodium, triclonide, trientine, trifenagrel, triflavin, triflocin, triflubazam, triflumidate, trifluoperazine hydrochloride, trifluperidol, triflupromazine, triflupromazine hydrochloride, trifluridine, trihexyphenidyl hydrochloride, trilostane, trimazosin hydrochloride, trimegestone, trimeprazine tartrate, trimethadione, trimethaphan camsylate, trimethobenzamide hydrochloride, trimethoprim, trimetozine, trimetrexate, trimipramine, trimoprostil, trimoxamine hydrochloride, triolein 1 125, triolein 1 131, trioxifene mesylate, tripamide, tripelennamine hydrochloride, triprolidine hydrochloride, triptorelin, trisulfapyrimidines, troclosene potassium, troglitazone, trolamine, troleandomycin, trombodipine, trometamol, tropanserin hydrochloride, tropicamide, tropine ester, tropisetron, trospectomycin, trovafloxacin, trovirdine, tryptophan, tuberculin, tubocurarine chloride, tubulozole hydrochloride, tucarcsol, tulobuterol, turosteride, tybamate, tylogenin, tyropanoate sodium, tyrosine, tyrothricin, tyrphostins, ubenimex, uldazepam, undecylenic acid, uracil mustard, urapidil, urea, uredepa, uridine triphosphate, urofollitropin, urokinase, ursodiol, valaciclovir, valine, valnoctamide, valproate sodium, valproic acid, valsartan (commercially available as DIOVAN® from Novartis Pharmaceuticals), vamicamide, vanadeine, vancomycin, vaminolol, vapiprost hydrochloride, vapreotide, vardenafil (commercially available as LEVITRA® from GlaxoSmithKline), variolin B, vasopressin, vecuronium bromide, velaresol, velnacrine maleate, venlafaxine, veradoline hydrochloride, veramine, verapamil hydrochloride, verdins, verilopam hydrochloride, verlukast, verofylline, veroxan, verteporfin, vesnarinone, vexibinol, vidarabine, vigabatrin, viloxazine hydrochloride, vinblastine sulfate, vinburnine citrate, vincofos, vinconate, vincristine sulfate, vindesine, vindesine sulfate, vinepidine sulfate, vinglycinate sulfate, vinleurosine sulfate, vinorelbine, vinpocetine, vintoperol, vinxaltine, vinzolidine sulfate, viprostol, virginiamycin, viridofulvin, viroxime, vitaxin, volazocine, voriconazole, vorozole, voxergolide, warfarin sodium, xamoterol, xanomeline, xanoxate sodium, xanthinol niacinate, xemilofiban, xenalipin, xenbucin, xilobam, ximoprofen, xipamide, xorphanol mesylate, xylamidine tosylate, xylazine hydrochloride, xylometazoline hydrochloride, xylose, yangambin, zabicipril, zacopride, zafirlukast, zalcitabine, zaleplon, zalospirone, zaltidine hydrochloride, zaltoprofen, zanamivir, zankiren, zanoterone, zantac, zarirlukast, zatebradine, zatosetron, zatosetron maleate, zenarestat, zenazocine mesylate, zeniplatin, zeranol, zidometacin, zidovudine, zifrosilone, zilantel, zilascorb, zileuton, zimeldine hydrochloride, zinc undecylenate, zindotrine, zinoconazole hydrochloride, zinostatin, zinterol hydrochloride, zinviroxime, ziprasidone, zobolt, zofenopril calcium, zofenoprilat, zolamine hydrochloride, zolazepam hydrochloride, zoledronie acid, zolertine hydrochloride, zolmitriptan, zolpidem, zomepirac sodium, zometapine, zoniclezole hydrochloride, zonisamide, zopiclone, zopolrestat, zorbamyciin, zorubicin hydrochloride, zotepine, zucapsaicin, JTT-501 (PNU-182716) (reglitazar), AR-H039122, MCC-555 (netoglitazone), AR-H049020 (tesaglitazar), CS-011 (CI-1037), GW-409544x, KRP-297, RG-12525, BM-15.2054, CLX-0940, CLX-0921, DRF-2189, GW-1929, GW-9820, LR-90, LY-510929, NIP-221, NIP-223, JTP-20993, LY 29311 Na, FK 614, BMS 298585, R 483, TAK 559, DRF 2725 (ragaglitazar), L-686398, L-168049, L-805645, L-054852, demethyl asteriquinone B1 (L-783281), L-363586, KRP-297, P32/98, CRE-16336, EML-1625, pharmaceutically acceptable salts thereof (e.g., Zn, Fe, Mg, K, Na, F, Cl, Br, I, acetate, diacetate, nitrate, nitrite, sulfate, sulfite, phosphate, and phosphite salts), pharmaceutically acceptable forms thereof with acid associates (e.g. HCl), and any combination thereof.

Examples of antibiotics that may be suitable use in conjunction with a drug delivery vehicle described herein may include, but are not limited to, to β-lactam antibiotics (e.g., benzathine penicillin, benzylpenicillin (penicillin G), phenoxymethylpenicillin (penicillin V), procaine penicillin, methicillin, oxacillin, nafcillin, cloxacillin, dicloxacillin, flucloxacillin, temocillin, amoxicillin, ampicillin, co-amoxiclav (amoxicillin+clavulanic acid), azlocillin, carbenicillin, ticarcillin, mezlocillin, piperacillin, cephalosporin, cephalexin, cephalothin, cefazolin, cefaclor, cefuroxime, cefamandole, cefotetan, cefoxitin, ceftriaxone, cefotaxime, cefpodoxime, cefixime, ceftazidime, cefepime, cefpirome, carbapenem, imipenem (with cilastatin), meropenem, ertapenem, faropenem, doripenem, aztreonam (commercially available as AZACTAM® from Bristol-Myers Squibb), tigemonam, nocardicin A, tabtoxinine-β-lactam, clavulanic acid, tazobactam, and sulbactam); aminoglycoside antibiotics (e.g., aminoglycoside, amikacin, apramycin, arbekacin, astromicin, bekanamycin, capreomycin, dibekacin, dihydrostreptomycin, elsamitrucin, G418, gentamicin, hygromycin B, isepamicin, kanamycin, kasugamycin, micronomicin, neomycin, netilmicin, paromomycin sulfate, ribostamycin, sisomicin, streptoduocin, streptomycin, tobramycin, verdamicin; sulfonamides such as sulfamethoxazole, sulfisomidine (also known as sulfaisodimidine), sulfacetamide, sulfadoxine, dichlorphenamide (DCP), and dorzolamide); quinolone antibiotics (e.g., cinobac, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, prulifloxacin, garenoxacin, and delafloxacin); oxazolidone antibiotics (e.g., linezolid, torezolid, eperezolid, posizolid, and radezolid), and any combination thereof.

Example of antifungals that may be suitable use in conjunction with a drug delivery vehicle described herein may include, but are not limited to, polyene antifungals (e.g., natamycin, rimocidin, filipin, nystatin, amphotericin B, candicin, and hamycin; imidazole antifungals such as miconazole (commercially available as MICATIN® from WellSpring Pharmaceutical Corporation), ketoconazole (commercially available as NIZORAL® from McNeil consumer Healthcare), clotrimazole (commercially available as LOTRAMIN® and LOTRAMIN AF® available from Merck and CANESTEN® available from Bayer), econazole, omoconazole, bifonazole, butoconazole, fenticonazole, isoconazole, oxiconazole, sertaconazole (commercially available as ERTACZO® from OrthoDematologics), sulconazole, and tioconazole; triazole antifungals such as fluconazole, itraconazole, isavuconazole, ravuconazole, posaconazole, voriconazole, terconazole, and albaconazole), thiazole antifungals (e.g., abafungin), allylamine antifungals (e.g., terbinafine (commercially available as LAMISIL® from Novartis Consumer Health, Inc.), naftifine (commercially available as NAFTIN® available from Merz Pharmaceuticals), and butenafine (commercially available as LOTRAMIN ULTRA® from Merck), echinocandin antifungals (e.g., anidulafungin, caspofungin, and micafungin), polygodial, benzoic acid, ciclopirox, tolnaftate (e.g., commercially available as TINACTIN® from MDS Consumer Care, Inc.), undecylenic acid, flucytosine, 5-fluorocytosine, griseofulvin, haloprogin, and any combination thereof.

Examples of active biologicals that may be suitable use in conjunction with a drug delivery vehicle described herein may include, but are not limited to, hormones (synthetic or natural and patient derived or otherwise), DNAs (synthetic or natural and patient derived or otherwise), RNAs (synthetic or natural and patient derived or otherwise), siRNAs (synthetic or natural and patient derived or otherwise), proteins and peptides (e.g., albumin, atrial natriuretic factor, renin, superoxide dismutase, a 1-antitrypsin, lung surfactant proteins, bacitracin, bestatin, cydosporine, delta sleep-inducing peptide (DSIP), endorphins, glucagon, gramicidin, melanocyte inhibiting factors, neurotensin, oxytocin, somostatin, terprotide, serum thymide factor, thymosin, DDAVP, dermorphin, Met-enkephalin, peptidoglycan, satietin, thymopentin, fibrin degradation product, des-enkephalin-α-endorphin, gonadotropin releasing hormone, leuprolide, α-MSH, and metkephamid), enzymes, nucleotides, oligionucleotides, antibodies, monoclonal antibodies, growth factors (e.g., epidermal growth factor (EGF), fibroblast growth factors, basic fibroblast growth factor (bFGF), nerve growth factor (NGF), bone derived growth factor (BDGF), transforming growth factors, transforming growth factor-β1 (TGF-β1), and human growth gormone (hGH)), viral surface antigens (e.g., adenoviruses, epstein-barr virus, hepatitis A virus, hepatitis B virus, herpes viruses, HIV-1, HIV-2, HTLV-III, influenza viruses, Japanese encephalitis virus, measles virus, papilloma viruses, paramyxoviruses, polio virus, rabies virus, rubella virus, vaccinia (smallpox) viruses, and yellow fever virus), bacterial surface antigens (e.g., bordetella pertussis, helicobacter pylorn, clostridium tetani, corynebacterium diphtheria, escherichia coli, haemophilus influenza, klebsiella species, legionella pneumophila, mycobacterium bovis, mycobacterium leprae, mycrobacterium tuberculosis, neisseria gonorrhoeae, neisseria meningitidis, proteus species, pseudomonas aeruginosa, salmonella species, shigella species, staphylococcus aureus, streptococcus pyogenes, vibrio cholera, and yersinia pestis), parasite surface antigens (e.g., plasmodium vivax—malaria, plasmodium falciparum—malaria, plasmodium ovale—malaria, plasmodium malariae—malaria, leishmania tropica—leishmaniasis, leishmania donovani, leishmaniasis, leishmania branziliensis—leishmaniasis, trypanosoma rhodescense—sleeping sickness, trypanosoma gambiense—sleeping sickness, trypanosoma cruzi—Chagas' disease, schistosoma mansoni—schistosomiasis, schistosomoma haematobium—schistomiasis, schistosoma japonicum—shichtomiasis, trichinella spiralis—trichinosis, stronglyloides duodenale—hookworm, ancyclostoma duodenale—hookworm, necator americanus—hookworm, wucheria bancrofti—filariasis, brugia malaya—filariasis, loa loa—filariasis, dipetalonema perstaris—filariasis, dracuncula medinensis—filariasis, and onchocerca volvulus—filariasis), immunogobulins (e.g., IgG, IgA, IgM, antirabies immunoglobulin, and antivaccinia immunoglobulin), and any combination thereof.

Examples of antitoxins that may be suitable use in conjunction with a drug delivery vehicle described herein may include, but are not limited to, botulinum antitoxin, diphtheria antitoxin, gas gangrene antitoxin, tetanus antitoxin, and any combination thereof.

Example of antigents that may be suitable use in conjunction with a drug delivery vehicle described herein may include, but are not limited to, foot and mouth disease, hormones and growth factors (e.g., follicle stimulating hormone, prolactin, angiogenin, epidermal growth factor, calcitonin, erythropoietin, thyrotropic releasing hormone, insulin, growth hormones, insulin-like growth factors 1 and 2, skeletal growth factor, human chorionic gonadotropin, luteinizing hormone, nerve growth factor, adrenocorticotropic hormone (ACTH), luteinizing hormone releasing hormone (LHRH), parathyroid hormone (PTH), thyrotropin releasing hormone (TRH), vasopressin, cholecystokinin, and corticotropin releasing hormone), cytokines (e.g., interferons, interleukins, colony stimulating factors, and tumor necrosis factors: fibrinolytic enzymes, such as urokinase, kidney plasminogen activator), clotting factors (e.g., Protein C, Factor VIII, Factor IX, Factor VII and Antithrombin III), and any combination thereof.

Examples of nutritional supplements that may be suitable use in conjunction with a drug delivery vehicle described herein may include, but are not limited to, vitamins, minerals, herbs, botanicals, amino acids, steroids, and the like.

Examples of nutraceuticals that may be suitable use in conjunction with a drug delivery vehicle described herein may include, but are not limited to, dietary supplements, botanicals, functional foods and extracts thereof, medicinal foods and extracts thereof, vitamins, minerals, co-enzyme Q, carnitine, multi-mineral formulas, gingseng, gingko biloba, saw palmetto, other plant-based supplements, probiotics, omega-3, canola and other oils, plant stanols, natural sweeteners, mushroom extracts, chocolate, chocolate extracts, grape extracts, berry extracts, super food extracts, quillaja molina extracts, plant extracts, yucca schidigera extract, bran, alanine, beta-carotene, carotenoids, arginin, vitamin A, asparagine, vitamin B-complex, aspartate, vitamin C, leucine, isoleucine, valine, vitamin D, citrulline, vitamin E, cysteine, vitamin K, glutamine, minerals, micro-nutrients, glutamic acid, calcium, glycine, chromium, histidine, copper, lysine, iodine, methionine, iron, ornithine, magnesium, phenylalanine, potassium, proline, selenium, serine, zinc, taurine, threonine, alpha lipoic acid, tryptophan, green tea extracts, tyrosine, essential fatty acids (EFA), whey protein, flax seed oil, and any combination thereof.

In some embodiments, a drug may be included (dispersed, dissolved, or otherwise) in a polymeric matrix described herein in an amount ranging from a lower limit of about 1%, 5%, 10%, 20%, or 30% by weight of the polymeric matrix to an upper limit of about 80%, 60%, 50%, 40%, 30%, or 20% by weight of the polymeric matrix, and wherein the amount of drug may range from any lower limit to any upper limit and encompass any subset therebetween. The amount of drug included in the polymeric matrix may depend on, inter alia, the composition of the polymeric matrix, the molecular weight of the drug, the interactions between the polymeric matrix and the drug, the desired drug dosage (described further herein), the configuration of the drug delivery vehicle (e.g., if a layer is used to modulate release from the vehicle versus release), and the like.

In some embodiments, a polymer matrix described herein that includes plasticized cellulose derivatives, plasticized starch derivatives, or both may be tacky at room temperature. As used herein, the term “tacky” refers to a composition that is tacky at room temperature to the extent that a 4 mil (the unit “mil” refers to a thousandth of an inch) coated paper backing sticks to the adhesive composition with no pressure applied (i.e., with only the weight of the 4 mil coated paper backing). In some instances, tacky compositions may include plasticized cellulose derivatives, plasticized starch derivatives, or both where the concentration of plasticized is about 40% or greater by weight of the cellulose derivative, starch derivative, or both.

In some embodiments, a polymer matrix described herein that includes plasticized cellulose derivatives, plasticized starch derivatives, or both may be non-tacky at room temperature. In some instances, tacky compositions may include plasticized cellulose derivatives, plasticized starch derivatives, or both where the concentration of plasticized is about 60% or less by weight of the cellulose derivative, starch derivative, or both.

The presence or absence of tack in the polymer matrix at room temperature may be modified by the concentration and composition of plasticizer, the composition of the cellulose or starch derivatives, the concentration of additional components like tackifiers, waxes, or additional polymers in the polymer matrix, and the like. Therefore, the foregoing plasticizer concentrations may be viewed as general guidelines and not limiting as to the presence or absence of tack in a polymer matrix.

Tailoring the glass transition temperature of a polymer matrix described herein that includes plasticized cellulose derivatives, plasticized starch derivatives, or both may alter the physical characteristics of the polymer matrix at ambient conditions (e.g., stiff or flexible, brittle or pliable, smooth or tacky, and the like, and any combination thereof). For example, a polymer matrix having no detectable glass transition temperature may be more tacky and flexible than a polymer matrix having a glass transition temperature. As used herein, the term “no detectable glass transition temperature” and derivatives thereof refers to material having no detectible heat flow event (as measured by differential scanning calorimeter (“DSC”)), which may be caused by the plasticized material having no glass transition temperature or the heat flow broadening to an extent that the glass transition temperature is not detectable. In another example, a polymer matrix having a higher glass transition temperature may be more stiff than a polymer matrix having a moderate to low glass transition temperature. In some embodiments, tailoring the glass transition temperature of a polymer matrix described herein may be achieved by, inter alia, changing the plasticizer concentration (e.g., increasing the concentration to decrease the glass transition temperature), changing the composition and or concentration of the plasticizer, the starch or cellulose source, the molecular weight the starch or cellulose derivatives, and the degree of substitution of the starch cellulose derivative (e.g., in some instances, increasing the degree of substitution to increase the glass transition temperature).

In some embodiments, a polymer matrix described herein that includes plasticized cellulose derivatives, plasticized starch derivatives, or both may have a glass transition temperature of about 190° C. or less. In some instances, a polymer matrix described herein that includes plasticized cellulose derivatives, plasticized starch derivatives, or both may have a glass transition temperature ranging from a lower limit of about −100° C., −75° C., −70° C., −30° C., 10° C., 75° C., or 120° C. to an upper limit of about 190° C., 150° C., 125° C., or 100° C., and wherein the glass transition temperature may range from any lower limit to any upper limit and encompass any subset therebetween. In some instances, a polymer matrix described herein may have no detectible glass transition temperature above about −100° C.

The glass transition temperature of a polymer matrix can be measured by either DSC or rheology. One skilled in the art with the benefit of this disclosure would understand that the glass transition temperature value may fall outside the preferred range described herein. Accordingly, within the scope of the embodiments described herein, the glass transition can be manipulated based on the composition and concentration of the components of the polymer matrix.

Tailoring the melt flow index of a polymer matrix described herein that includes plasticized cellulose derivatives, plasticized starch derivatives, or both may be useful in the production of drug delivery vehicles. For example, a higher melt flow index composition may be useful forming portions of a drug delivery device where flow at moderate temperature is advantageous (e.g., when used in conjunction with drugs that are temperature sensitive). Additionally, the ability to tailor the melt flow index may be advantageous when forming drug delivery vehicles with a second portion that includes a polymer matrix without a plasticized cellulose derivative or a plasticized starch derivative, where processing or forming of the drug delivery vehicle is aided by having similar melt flow indices for the two portions.

In some embodiments, tailoring the melt flow index of a polymer matrix described herein that includes plasticized cellulose derivatives, plasticized starch derivatives, or both may be achieved by, inter alia, changing the plasticizer composition, changing the plasticizer concentration (e.g., increasing the concentration to increase the melt flow index), changing the molecular weight of the starch derivative or cellulose derivative (e.g., decreasing molecular weight to increase the melt flow index), changing the starch or cellulose source, and changing the composition or concentration of additives.

In some embodiments, a polymer matrix described herein that includes plasticized cellulose derivatives may have a melt flow index (with a 300 sec melt time) ranging from a lower limit of about 0 g/10 min, 5 g/10 min, 25 g/10 min, 29 g/10 min, 35 g/10 min, or 40 g/10 min (at 150° C./0.5 kg measured in accordance with ASTM D1238) to an upper limit of about 150 g/10 min, 125 g/10 min, 100 g/10 min, 80 g/10 min, 70 g/10 min, 60 g/10 min, 50 g/10 min, or 40 g/10 min (at 150° C./0.5 kg measured in accordance with ASTM D1238), and wherein the melt flow index may range from any lower limit to any upper limit and encompass any subset therebetween. In some instances where the melt flow index at 150° C./500 g is greater than 150 g/10 min, the melt flow index may be measured at 150° C./100 g and range from a lower limit of about 5 g/10 min, 25 g/10 min, 29 g/10 min, 35 g/10 min, or 40 g/10 min (at 150° C./100 g measured in accordance with ASTM D1238) to an upper limit of about 86 g/10 min, 80 g/10 min, 70 g/10 min, 60 g/10 min, 50 g/10 min, or 40 g/10 min (at 150° C./100 g measured in accordance with ASTM D1238), and wherein the melt flow index may range from any lower limit to any upper limit and encompass any subset therebetween.

In some instances where starch derivatives are used, measuring melt flow index at 150° C. may not be appropriate because starch derivatives may decompose to some degree at that temperature. Accordingly, the melt flow index may be measured at 125° C. In some embodiments, a polymer matrix described herein that includes plasticized starch derivatives may have a melt flow index (with a 300 sec melt time) ranging from a lower limit of about 0 g/10 min, 5 g/10 min, 25 g/10 min, 29 g/10 min, 35 g/10 min, or 40 g/10 min (at 125° C./0.5 kg measured in accordance with ASTM D1238) to an upper limit of about 150 g/10 min, 125 g/10 min, 100 g/10 min, 80 g/10 min, 70 g/10 min, 60 g/10 min, 50 g/10 min, or 40 g/10 min (at 125° C./0.5 kg measured in accordance with ASTM D1238), and wherein the melt flow index may range from any lower limit to any upper limit and encompass any subset therebetween. In some instances where the melt flow index at 125° C./500 g is greater than 150 g/10 min, the melt flow index may be measured at 125° C./100 g and range from a lower limit of about 5 g/10 min, 25 g/10 min, 29 g/10 min, 35 g/10 min, or 40 g/10 min (at 125° C./100 g measured in accordance with ASTM D1238) to an upper limit of about 500 g/10 min, 400 g/10 min, 300 g/10 min, 200 g/10 min, 100 g/10 min, 86 g/10 min, 80 g/10 min, 70 g/10 min, 60 g/10 min, 50 g/10 min, or 40 g/10 min (at 125° C./100 g measured in accordance with ASTM D1238), and wherein the melt flow index may range from any lower limit to any upper limit and encompass any subset therebetween.

It should be noted that the melt flow index of a polymer matrix described herein that includes plasticized cellulose derivatives, plasticized starch derivatives, or both may fall outside the ranges described herein depending on the composition and concentration of the components of the polymer matrix.

Tailoring the adhesive strength of a polymer matrix described herein that includes plasticized cellulose derivatives, plasticized starch derivatives, or both may be useful in producing various portions of a drug delivery vehicle. For example, a lower adhesive strength may be useful in an adhesive portion that temporarily sticks to a patient (e.g., an adhesive 102 of a patch 100 illustrated in FIG. 1). Higher adhesive strength may be useful when a polymer matrix described herein that includes plasticized cellulose derivatives, plasticized starch derivatives, or both should be permanently adhered to another surface (e.g., an intermediate layer 106 of a patch 100 illustrated in FIG. 1).

In some embodiments, tailoring the adhesive strength of a polymer matrix described herein that includes plasticized cellulose derivatives, plasticized starch derivatives, or both may be achieved by, inter alia, changing the plasticizer composition, changing the plasticizer concentration (e.g., increasing the concentration to decrease the adhesive strength), changing the molecular weight of the starch derivative or cellulose derivative (e.g., decreasing molecular weight to decrease the adhesive strength), and changing the composition or concentration of additives (e.g., increasing crosslinker, tackifier, and/or filler concentration to increase the adhesive strength).

Adhesive strength may be measured by peel adhesion and/or lap shear strength.

In some embodiments, a polymer matrix described herein that includes plasticized cellulose derivatives, plasticized starch derivatives, or both may have a peel adhesion (using with a 4 mil coated paper backing) ranging from a lower limit of about 0.1 lb/in, 0.25 lb/in, 0.5 lb/in, 1 lb/in, 2 lb/in, 3 lb/in, 4 lb/in, or 5 lb/in to an 25 lb/in, 20 lb/in, 15 lb/in, or 10 lb/in, and wherein the peel adhesion may range from any lower limit to any upper limit and encompass any subset therebetween. Depending on the substrate, in some instances, the substrate may fail (e.g., tear) before the polymer matrix. The peel adhesion can be measured by ASTM 3330/D Method A (Standard test method for peel adhesion of PSA tape (180° Peel)) and tested on a surface of interest (e.g., corrugated cardboard, glass, stainless steel panels). Test method A gives a measure of the adherence, when peeled at 180° angle, to a standard steel panel or to other surfaces of interest (e.g., corrugated board or glass) for a single-coated tape. This test method provides a means for assessing the uniformity of the adhesion of a given polymer matrix. In this method, a strip is applied to a standard test panel (or other surface of interest) with controlled pressure. The tape is peeled from the panel at 180° angle at a specified rate with a 1 kN load cell, during which the force required to effect peel is measured. One skilled in the art with the benefit of this disclosure would understand that the peel adhesion described herein may have a range of peel adhesion depending on the polymer matrix, the coat weight of the polymer matrix, and the substrate to which it is adhered to.

The lap shear strength of a polymer matrix described herein that includes plasticized cellulose derivatives, plasticized starch derivatives, or both can be measured by testing lap shears by tension loading with a 1 kN load cell by a method that includes placing a specimen (two substrates with a 1 inch by 1 inch overlap and 3 mm thick glue line) in the grips of the testing machine so that each end of the specimen is in contact with the grip assemble, applying the loading immediately to the specimen at the rate of 800 lb force of shear per min, and continuing the load to failure of the polymer matrix or substrate. Polymer matrix failure is recorded as the lap shear strength, and substrate failure is recorded as substrate failure. In some instances, substrate failure for a 4 mil coated paper has been observed at about 17 kgf. This value may change depending on the substrate and size of the glue line.

In some embodiments, a polymer matrix described herein that includes plasticized cellulose derivatives, plasticized starch derivatives, or both may have a lap shear strength (using with a 4 mil coated paper backing) ranging from a lower limit of about 0.2 kgf, 0.5 kgf, 1 kgf, 2 kgf, 4 kgf, or 6 kgf to an upper limit of about 17 kgf, 15 kgf, 10 kgf, 8 kgf, 6 kgf, or 4 kgf, and wherein the lap shear strength may range from any lower limit to any upper limit and encompass any subset therebetween. In some instances, the 4 mil coated paper may fail before the polymer matrix. In some embodiments, a polymer matrix described herein that includes plasticized cellulose derivatives, plasticized starch derivatives, or both may have a lap shear strength (using an aluminum or stainless steel substrate) ranging from a lower limit of about 0.2 kgf, 0.5 kgf, 1 kgf, 2 kgf, 5 kgf, or 10 kgf to an upper limit of about 30 kgf, 25 kgf, 20 kgf, 15 kgf, or 10 kgf, and wherein the lap shear strength may range from any lower limit to any upper limit and encompass any subset therebetween.

The clarity of a polymer matrix described herein that includes plasticized cellulose derivatives, plasticized starch derivatives, or both may be important in some applications. For example, dermal patches may be produced with high clarity (or low haze) so as to be less obvious when exposed. In some embodiments, tailoring the clarity of a polymer matrix described herein that includes plasticized cellulose derivatives, plasticized starch derivatives, or both may be achieved by, inter alia, changing the source of starch, changing the plasticizer composition and/or concentration (e.g., increasing the concentration to increase the clarity/decrease the haze) and changing the composition and/or concentration of additives (e.g., increasing the filler concentration to decrease the clarity/increase the haze).

In some embodiments, a polymer matrix described herein that includes plasticized cellulose derivatives, plasticized starch derivatives, or both may have a haze ranging from a lower limit of about 3, 5, 15, 20, or 25 to an upper limit of about 100 (i.e., intentionally opaque), 85, 70, 60, or 40, and wherein the haze may range from any lower limit to any upper limit and encompass any subset therebetween. The haze of an polymer matrix can be measured with properly sized specimens substantially plane-parallel surfaces (e.g., flat without wrinkling) free of dust, scratches, and particles of about 0.85 mm in thickness using an UtraScan Pro from Hunter Lab with D65 Illuminant/10° observer. One skilled in the art with the benefit of this disclosure would understand that the haze value may fall outside the preferred ranges described herein for different thickness of the polymer matrix. In some instances, the haze value may be significantly larger than the preferred ranges above (e.g., about 100) when additives like titanium dioxide are used in significant quantities to produce an opaque polymer matrix. Additionally, pigments and dyes may affect the haze of the polymer matrix.

Drug delivery vehicles may have a variety of configurations for administration to a patient orally (e.g., pills, tablets, and the like), subdermally (e.g., subdermal implants), transdermally (e.g., patches, bandages, and the like), transmucosally (e.g., oromucosal inserts, intrauterine devices, intravaginal rings, dental fibers, and the like), and/or as a part of an implantable medical device. As used herein, the term “subject” and “patient” are used interchangeably herein and refer to both human and nonhuman animals and insects. The term “nonhuman animals” as used herein includes all vertebrates (e.g., mammals and non-mammals) such as nonhuman primates, mice, rats, sheep, dogs, cats, horses, cows, chickens, amphibians, fish, reptiles, and the like. The term “insects” as used herein includes all arthropods (e.g., bees, flies, Drosophila flies, beetles, spiders, and the like).

A typical dosage of drugs may range from about 0.001 mg/kg to about 1000 mg/kg, preferably from about 0.01 mg/kg to about 100 mg/kg, and more preferably from about 0.10 mg/kg to about 20 mg/kg, relative to weight of the patient. In some instances, low dose delivery may be useful for treating addiction.

One skilled in the art should understand the dose and/or combination of drugs should be chosen so as to minimize adverse interactions. Further, the inclusion of plasticized cellulose derivatives, plasticized starch derivatives, or both in drug delivery vehicles described herein may allow for combinations of drugs not previously realized by exploiting the hydrophilicity of the plasticized derivatives, the potentially high loading of the polymer matrix, and the combination of the hydrophilic plasticized derivatives with traditional, more hydrophobic drug delivery polymers like ethylene vinyl acetate copolymer.

In some embodiments, a drug delivery vehicle described herein may be a component of a kit for the treatment or prevention of a disease or condition in a patient. In some embodiments, a kit may include a set of instructions and at least one controlled release vehicle of the present invention. In some embodiments, a kit may include a set of instructions and an article comprising at least one drug delivery vehicle described herein.

FIG. 1 illustrates a cross-section of a dermal patch 100 with an adhesive 102 on the skin side 104 and an intermediate layer 106 disposed between the adhesive 102 and a backing layer 108, which is substantially impermeable to the drug. Generally, the intermediate layer 106 comprises a drug and may generally act as a drug reservoir. The adhesive 102 is permeable to the drug so as to allow for the drug to diffuse from the intermediate layer 106 to the patient. In some instances, the adhesive 102 may modulate dosage of the drug to the patient. In some instances, a modulation layer (not shown) may be disposed between the intermediate layer 106 and the adhesive 102 so as to modulate dosage of the drug to the patient. In some embodiments, the adhesive 102 may be formed of a polymer matrix that includes plasticized cellulose derivatives, plasticized starch derivatives, or both. In some embodiments, the intermediate layer 106 may be formed of a polymer matrix that includes plasticized cellulose derivatives, plasticized starch derivatives, or both. In some embodiments, the modulation layer may be formed of a polymer matrix that includes plasticized cellulose derivatives, plasticized starch derivatives, or both. In some instances, a combination of two or more of the foregoing may be used to produce the dermal patch 100.

Exemplary drugs that may be useful in patches and other similar drug delivery vehicles may include, but are not limited to, hormones (e.g., ethinyl estradiol, etonogestrel, progesterone, levonorgestrel), antiretroviral drugs (e.g., tenofovir), nicotine, opiates, analgesics (e.g., acetylsalicylic acid, acetaminophen, naproxen, and morphine), anti-bacterial agents (e.g., penicillin, neomycin, and netilmicin), anti-inflammatory drugs (e.g., acetylsalicylic acid, acetaminophen, and naproxen), and the like, and combinations thereof.

FIG. 2 illustrates a cross-section of an intravaginal ring 200 with a core 202 and a skin 204. In some instances, additional layers (not shown) may be disposed between the core 202 and the skin 204. Generally, the skin 204 modulates dosage of the drug to the patient, while the core 202 provides for a drug reservoir. Intermediate layers may behave similar to one of the core 202 or the skin 204. In some embodiments, the core 202 may be formed of a polymer matrix that includes plasticized cellulose derivatives, plasticized starch derivatives, or both. In some embodiments, the skin 204 may be formed of a polymer matrix that includes plasticized cellulose derivatives, plasticized starch derivatives, or both. In some embodiments, the intermediate layers may be formed of a polymer matrix that includes plasticized cellulose derivatives, plasticized starch derivatives, or both. In some instances, a combination of two or more of the foregoing may be used to produce the intravaginal ring 200.

In some embodiments, other drug delivery vehicles (e.g., rod implants, intravaginal springs or coils, intrauterine devices, and the like) may have cross-sectional compositions similar to that of the intravaginal ring 200 of FIG. 2.

Exemplary drugs that may be useful in intravaginal rings and other similar drug delivery vehicles may include, but are not limited to, hormones (e.g., ethinyl estradiol, etonogestrel, progesterone, levonorgestrel), antiretroviral drugs (e.g., tenofovir), and the like, and combinations thereof.

In another example, a drug delivery vehicle may be a bandage where a polymer matrix described herein may be a coating on at least one side of the bandage. In some instances, the polymer matrix disposed on the bandage may be tacky to provide adhesion to itself or the treatment area. For example, a long, gauze-like bandage may be tacky on at least one side to provide adhesion when wrapping a treatment area. In another example, a bandage may be shaped like an article of clothing or a sleeve that provides compression. In some instances, a polymer matrix described herein includes plasticized cellulose derivatives, plasticized starch derivatives, or both may have sufficient pliability to minimize interference with such compression and be useful in providing drug delivery to the treatment area.

Exemplary drugs that may be useful in banage drug delivery vehicles may include, but are not limited to, analgesics (e.g., acetylsalicylic acid, acetaminophen, naproxen, and morphine), anti-bacterial agents (e.g., penicillin, neomycin, and netilmicin), anti-inflammatory drugs (e.g., acetylsalicylic acid, acetaminophen, and naproxen), and the like, and combinations thereof.

Drug delivery vehicles may include at least one polymer matrix described herein that includes plasticized cellulose derivatives, plasticized starch derivatives, or both. In some instances, drug delivery vehicles may also include a polymer matrix that does not include plasticized cellulose derivatives or plasticized starch derivatives. For example, the additional polymers described herein for blending in the polymer matrix may be used in a portion of a drug delivery vehicle without being blended with plasticized cellulose derivatives or plasticized starch derivatives.

A polymer matrix described herein may be formed by blending the components of the polymer matrix. In some instances, blending may involve high-shear mixing processes. In some instances, blending may be performed at an elevated temperature (e.g., a temperature above room temperature). In some instances, the components of the polymer matrix may be heated before and during blending. Some embodiments may involve a combination of the foregoing.

Drug delivery vehicles or portions thereof described herein may be produced by extrusion, injection molding, over molding, compression molding, laminating, casting, spraying, and the like, any hybrid thereof, and any combination thereof.

For example, a polymer melt comprising a polymer matrix described herein may be extruded into a desired shape (e.g., a rod or a sheet). In another example, two or more polymer melts described herein may be coextruded to form a core/skin cross-section (e.g., as illustrated in FIG. 2) or a flat, layered cross-section (e.g., as illustrated in FIG. 1).

In some instances, individual layers of a drug delivery vehicle may be formed (e.g., by extrusion or coextrusion) and laminated together. In some instances, tie layers may be used enhance the adherence adjacent layers of drug delivery vehicle. That is, a tie layer may be interposed between two layers where the two layers do not sufficiently adhere to each other but rather individually adhere better to the tie layer.

In some instances, the shape formed by extrusion may be the drug delivery vehicle (e.g., a pill or an implantable rod). In some instances, the shape formed by extrusion may be formed into the drug delivery vehicle (e.g., by welding the ends of the rod into a ring shape to yield an intravaginal ring).

In some instances, individual layers of a drug delivery vehicle may be formed and laminated together. In some instances, tie layers may be used to adhere adjacent layers of drug delivery vehicle.

In some instances, additional layers may be applied after extrusion. For example, a backing and intermediate layer of a patch may be coextruded or laminated together, and then an adhesive layer may be applied to the intermediate layer.

In some embodiments, incorporation of a drug in a desired portion of the drug delivery vehicle may be achieved by including the drug in the polymer melt (e.g., by compounding the drug with the polymer matrix components). In such instances, the temperature of compounding and extrusion should be controlled to mitigate thermal degradation of the drug.

In some embodiments, incorporation of a drug in a desired portion of the drug delivery vehicle may be achieved by adsorbing the drug into the portion of the drug delivery vehicle after production. For example, a sheet or layer may be formed of a polymer matrix, which may then be exposed to a drug (e.g., soaking, spraying, etc.) for adsorption into the polymer matrix.

The dimensions or sized of the drug delivery vehicle and portions thereof may be configured as needed for drug delivery vehicle implementation, drug release rates, and the like. For example, the shape of an intravaginal ring or intrauterine devices may be sized and shaped accordingly where the thickness of individual portions/polymeric matrices in the cross-section (e.g., as illustrated in FIG. 2) may be sized for a desired release rate, which may depend on the composition of each polymer matrix, the composition of the drug, and the like. One skilled in the art could determine the dimensions of a drug delivery vehicle and portions thereof without undo experimentation.

Embodiments disclosed herein include:

A. a drug delivery vehicle that includes an intermediate layer comprising a drug disposed between an adhesive and a backing, wherein one of the intermediate layer and the adhesive are formed by a polymeric matrix that comprises a plasticizer and at least one selected from the group consisting of: a cellulose ester, a cellulose ether, a starch ester, a starch ether, and a combination thereof;

B. a drug delivery vehicle that includes a core polymer matrix comprising a drug and encompassed by a skin polymer matrix, wherein the core polymer matrix or the skin polymer matrix comprises a plasticizer and at least one selected from the group consisting of: a cellulose ester, a cellulose ether, a starch ester, a starch ether, and a combination thereof; and

C. a drug delivery vehicle that includes a bandage with a polymer matrix disposed thereon, wherein the polymer matrix comprises a drug, a plasticizer, and at least one selected from the group consisting of: a cellulose ester, a cellulose ether, a starch ester, a starch ether, and a combination thereof.

Each of embodiments A, B, and C may have one or more of the following additional elements in any combination: Element 1: wherein the polymeric matrix comprises the at least one selected from the group consisting of: the cellulose ester, the cellulose ether, the starch ester, the starch ether, and the combination thereof at about 10% to about 80% by weight of the polymeric matrix; Element 2: wherein the polymeric matrix further comprises at least one selected from the group consisting of: a polyolefin, an ethylene copolymer, a thermoplastic polyurethane, an acrylic acid polymer, polytetrafluoroethylene, an ethylene vinyl acetate copolymer derivative, a polyester, a polysiloxane, polybutadiene, polyisoprene, poly(methacrylate), poly(methyl methacrylate), a styrene-butadiene-styrene block copolymer, poly(hydroxyethylmethacrylate), poly(vinyl chloride), poly(vinyl acetate), a polyether, a polyacrylonitrile, a polyethylene glycol, polymethylpentene, polybutadiene, polyhydroxy alkanoates, poly(lactic acid), poly(glycolic acid), acrylic acid-based polymers, a methacrylic acid based polymer, a polyanhydride, a polyorthoester, cross-linked poly(vinyl alcohol), neoprene rubber, butyl rubber, polyvinyl acetate phthalate, polyvinyl acetate, shellac, zein, polyethylene oxide, ethylene oxide-propylene oxide copolymers, polyethylene-polypropylene glycol, carbomer, polycarbophil, chitosan, polyvinyl pyrrolidone, poly(vinyl alcohol), a polyethyleneimine, a polyacrylate, a polyacrylamide, a polymethacrylamide, a polyphosphazine, a polyoxazolidine, a polyhydroxyalkylcarboxylic acid, an alginic acid, natural gums, povidone, gelatin, and the like, any derivative thereof, any copolymer thereof, any blend polymer thereof, and combinations thereof; Element 3: wherein the polymeric matrix consists essentially of the plasticizer and the at least one selected from the group consisting of: the cellulose ester, the cellulose ether, the starch ester, the starch ether, and the combination thereof; Element 4: wherein the polymeric matrix further comprises ethylene vinyl acetate copolymer; Element 5: wherein the polymer matrix is tacky at room temperature; Element 6: wherein the plasticizer is in the polymeric matrix at about 15% or greater by weight of the polymeric matrix; and Element 7: wherein the plasticizer comprises at least one plasticizer described herein; and Element 8: wherein the plasticizer comprises a mixture of two or more plasticizers.

By way of non-limiting example, exemplary combinations applicable to A, B, C include: one of Elements 2-4 in combination with Element 5 and optionally Element 6; Element 1 in combination with one of Elements 2-4 and optionally Element 6; and Element 7 in combination with Element 8 and optionally element 6.

In some instances, the drug delivery device of Embodiment A may be configured such that the adhesive comprises the polymeric matrix and the plasticizer is in the polymeric matrix at about 15% or greater by weight of the polymeric matrix. In some instances, the drug delivery device of Embodiment A may be configured such that the intermediate layer comprises the polymeric matrix and the polymeric matrix consists essentially of the plasticizer, the drug, and the at least one selected from the group consisting of: a cellulose ester, a cellulose ether, a starch ester, a starch ether, and a combination thereof. In some instances, the drug delivery device of Embodiment B may be dimensioned for use as an intravaginal ring. In some instances, the drug delivery device of Embodiment B may be dimensioned for use as a rod-shaped implant.

To facilitate a better understanding of the present invention, the following examples of preferred or representative embodiments are given. In no way should the following examples be read to limit, or to define, the scope of the invention.

### Examples

### Example 1

A plurality of adhesive samples was prepared by compounding cellulose acetate and a plasticizer in the amounts and compositions detailed in Table 1. The cellulose acetates tested were CA-1 having a degree of substitution of about 2.5 and a molecular weight (Mₙ) of about 78,000, CA-2 having a degree of substitution of about 2.4 and a Mₙ of about 44,000, and CA-3 having a degree of substitution of about 2.4 and a Mₙ of about 62,000. The characteristics of the adhesive samples and control cellulose acetate samples without plasticizer were measured and are reported in Table 2.

TABLE 1

| | Cellulose Acetate | Plasticizer | |
|---|---|---|---|
| Sample | Composition | Composition | Wt % Plasticizer |
| CA-1 | CA-1 | | 0 |
| PCA-1 | CA-1 | triacetin | 20 |
| PCA-2 | CA-1 | triacetin | 40 |
| PCA-3 | CA-1 | triacetin | 60 |
| PCA-4 | CA-1 | tributyl phosphate | 20 |
| PCA-5 | CA-1 | tributyl phosphate | 40 |
| PCA-6 | CA-1 | tributyl phosphate | 60 |
| CA-2 | CA-2 | | 0 |
| PCA-7 | CA-2 | triacetin | 60 |
| PCA-8 | CA-2 | triacetin | 70 |
| PCA-9 | CA-2 | tributyl phosphate | 60 |
| CA-3 | CA-3 | | 0 |
| PCA-10 | CA-3 | triacetin | 60 |
| PCA-11 | CA-2 | eugenol | 50 |
| PCA-12 | CA-2 | ethylvanillin | 50 |
| | | triacetin and | 62 (92:8 triacetin: |
| PCA-13 | CA-2 | ethylvanillin | ethylvanillin) |
| PCA-14 | CA-2 | triacetin and | 64 (84:16) |
| | | ethylvanillin | |
| PCA-15 | CA-2 | acetovanillone | 50 |
| PCA-16 | CA-2 | triacetin and | 62 (92:8) |
| | | acetovanillone | |

TABLE 2

| | | | | Complex |
|---|---|---|---|---|
| | | | | Viscosity³ |
| Sample | Description | MP¹ (° C.) | T_{g}² (° C.) | (Pa*s) |
| CA-1 | white flake | | 167-207⁴ | |
| PCA-1 | clear; stiff; brittle | | 80 | 93,777 |
| PCA-2 | clear; flexible; tacky | | −55 | 7,187 |
| PCA-3 | clear; flexible; | 150¹ | −53 | 2,417 |
| | stretchy; very tacky | | | |
| PCA-4 | clear; stiff; brittle | 166² | none | 122,456 |
| | | | detected | |
| PCA-5 | clear; stiff with | 180² | 14 | 56,004 |
| | some flexibility | | | |
| PCA-6 | clear; flexible; tacky | 180¹ | 12 | 13,661 |
| CA-2 | white flake | | 167-207⁴ | |
| PCA-7 | clear; flexible; | | −44 | 4,037 |
| | stretchy; tacky | | | |
| PCA-8 | gel-like | | -61 | 4,037 |
| PCA-9 | clear; flexible | | 15 | 23,230 |
| CA-3 | white flake | | 167-207⁴ | |
| PCA-10 | clear; flexible; | | −57 | |
| | stretchy; tacky | | | |
| PCA-11 | clear; coloured; | | −43 | |
| | tacky; flexible | | | |
| PCA-12 | hard; glass-like; | | −35 | |
| | clear-yellow | | | |
| PCA-13 | clear; flexible | | −53 | |
| PCA-14 | clear; flexible | | −51 | |
| PCA-15 | hard; glass-like; | | −34 | |
| | clear yellow | | | |
| PCA-16 | clear; flexible | | −52 | |

¹Flow onset point as measured by visual inspection upon heating.
²Glass transition temperature as measured by TA Instruments DSC Q2000.
³Complex viscosity at 140° C. by TA Instruments Rheometer Discovery HR-2.
⁴Literature values for cellulose acetate.

### Example 2

Samples PCA-3, PCA-6, PCA-7, and PCA-9 were tested for adherence between a glass surface and a cardboard surface. A portion of the sample was added to a glass slide and heated to between 60° C. and 100° C. Then a piece of cardboard was applied to the adhesive, which was then allowed to cool. The cardboard piece was then peeled from the glass slide.

Adhesion was achieved in all samples. Upon trying to separate the two substrates, the cardboard pieces adhered with samples PCA-3, PCA-6, and PCA-7 were unable to be peeled without rupturing the cardboard. The cardboard piece adhered with sample PCA-9 was able to be cleanly peeled from the glass slide.

### Example 3

PCA-7 was tested for thermal stability by storing in a freezer for over 24 hours two paper surfaces glued together. Once warmed to room temperature, the paper surfaces were manually pulled and remained adhered together. Further, the seam where the PCA-7 adhered to the two paper surfaces remained flexible after the temperature cycling. This example appears to demonstrate, to at least some extent, the temperature stability of at least some of the adhesive described herein.

### Example 4

Mixes of CA with intrinsic viscosities from 0.8 to 1.6 and triacetin content to CA ratio of 1:1 and 0.8:1 were prepared. The mixes were analyzed for the changes in melt temperature as a function of intrinsic viscosity. As shown in FIG. 2, a substantially linear relationship was observed where increased intrinsic viscosity yields a linear increase in melt temperature. Further, a higher plasticizer concentration yields a lower melt temperature at the same intrinsic viscosity. This example appears to demonstrate the ability to tailor the flow onset temperature response by controlling intrinsic viscosity or plasticizer concentration of PCA.

### Example 5

An adhesive melt was prepared by compounding cellulose diacetate (40% by weight of the adhesive melt) with triacetin plasticizer (60% by weight of the adhesive melt) and placing the compounded mixture in an oven for about 5 min at 140° C. The adhesive melt was then coated to one surface/side of a card-stock paper substrate and allowed to cool so as to yield a laminate film on the paper surface. The coated substrate was subjected to an additional heating step at 140° C. for 2-3 minutes. The laminate film was glossy, flexible, and well adhered to the surface precluding the need for both film and laminating adhesive.

### Example 6

A plurality of adhesive samples were prepared by compounding cellulose acetate and a plasticizer in the amounts and compositions detailed in Table 3. The cellulose acetates tested were CA-2 from Example 1 and CA-4 having a degree of substitution of about 2.4, a Mₙ of about 60,000, and an intrinsic viscosity of about 1.36 dL/g. The characteristics of the adhesive samples and control cellulose acetate samples without plasticizer were measured and are reported in Table 4.

TABLE 3

| | Cellulose Acetate | | Wt % |
|---|---|---|---|
| Sample | Composition | Plasticizer Composition | Plasticizer |
| PCA-17 | CA-4 | diacetin | 60 |
| PCA-18 | CA-4 | triacetin | 60 |
| PCA-19 | CA-1 | diacetin | 60 |
| PCA-20 | CA-4 | diacetin and | 62 (92:8 |
| | | acetylsalicylic acid | diacetin: |
| | | | acetylsalicylic |
| | | | acid) |
| PCA-21 | CA-4 | triacetin and | 62 (92:8) |
| | | acetylsalicylic acid | |
| PCA-22 | CA-4 | triacetin and butylated | 62 (92:8) |
| | | hydroxyltoluene | |
| PCA-23 | CA-4 | diacetin and butylated | 62 (92:8) |
| | | hydroxytoluene | |
| PCA-24 | CA-4 | triacetin and butylated | 62 (92:8) |
| | | hydroxylanisol | |
| PCA-25 | CA-4 | diacetin and butylated | 62 (92:8) |
| | | hydroxyanisol | |
| PCA-26 | CA-4 | triacetin and benzoic | 62 (92:8) |
| | | acid | |
| PCA-27 | CA-4 | diacetin and benzoic | 62 (92:8) |
| | | acid | |
| PCA-28 | CA-4 | triacetin and | 62 (92:8) |
| | | SYLVATAC ® RE85 | |
| PCA-29 | CA-4 | diacetin and | 62 (92:8) |
| | | SYLVATAC ® RE85 | |
| PCA-30 | CA-4 | triacetin and | 62 (92:8) |
| | | SYLVALITE ® RE100 | |
| PCA-31 | CA-4 | diacetin and | 62 (92:8) |
| | | SYLVALITE ® RE100 | |
| PCA-32 | CA-2 | triacetin and | 62 (92:8) |
| | | SYLVATAC ® RE85 | |
| PCA-33 | CA-2 | triacetin and | 62 (92:8) |
| | | SYLVALITE ® RE100 | |
| PCA-34 | CA-4 | diacetin and ethyl | 62 (92:8) |
| | | vanillin | |
| PCA-35 | CA-2 | triacetin and ethyl | 62 (92:8) |
| | | vanillin | |
| PCA-36 | CA-4 | diacetin and salicylic | 62 (92:8) |
| | | acid | |
| PCA-37 | CA-4 | triacetin and xylitol | 62 (92:8) |
| PCA-38 | CA-4 | triacetin and sorbitol | 62 (92:8) |
| PCA-39 | CA-2 | triacetin and xylitol | 62 (92:8) |
| PCA-40 | CA-2 | triacetin and sorbitol | 62 (92:8) |
| PCA-41 | CA-2 | triacetin and gamma | 62 (92:8) |
| | | valerolactone | |

TABLE 4

| | | | Melt Flow Index⁶ |
|---|---|---|---|
| Sample | Description | T_{g}⁵ (° C.) | (g/10 min) |
| CA-4 | white flake | 167-207⁷ | |
| PCA-17 | clear; flexible; stretchy | −69 | 40 |
| PCA-18 | clear; flexible; stretchy | −53 | 31 |
| PCA-19 | clear; hard | −66 | 16 |
| PCA-20 | clear; flexible; | −66 | 57 |
| | stretchy; tacky | | |
| PCA-21 | clear; flexible; | −54 | 49 |
| | stretchy; tacky | | |
| PCA-22 | clear-yellow; flexible; | −55 | |
| | stretchy | | |
| PCA-23 | clear-yellow; flexible; | −63 | 56 |
| | stretchy | | |
| PCA-24 | clear-yellow; flexible; | −55 | |
| | stretchy; tacky | | |
| PCA-25 | clear-yellow; flexible; | −62 | 46 |
| | stretchy; tacky | | |
| PCA-26 | clear-yellow; flexible; | −56 | 51 |
| | stretchy; tacky | | |
| PCA-27 | clear-yellow; flexible; | −59 | 67 |
| | stretchy; tacky | | |
| PCA-28 | yellow; flexible | −54 | 45 |
| PCA-29 | yellow; flexible | −61 | 38 |
| PCA-30 | white; flexible; | −54 | 68 |
| | stretchy; tacky | | |
| PCA-31 | white; flexible; | | 47 |
| | stretchy; tacky | | |
| PCA-32 | white; flexible; | −53 | 27⁸ |
| | stretchy; tacky | | |
| PCA-33 | white; flexible; | −53 | 21⁸ |
| | stretchy; tacky | | |
| PCA-34 | clear-yellow; flexible; | −68 | 81 |
| | stretchy; tacky | | |
| PCA-35 | clear; flexible; | −54 | 34⁸ |
| | stretchy; tacky | | |
| PCA-36 | clear-yellow; flexible; | −63 | 80 |
| | stretchy; tacky | | |
| PCA-37 | clear; flexible | −51 | 44 |
| PCA-38 | clear; flexible | −56 | 41 |
| PCA-39 | clear; flexible | −55 | |
| PCA-40 | clear; flexible | −54 | |

⁵Glass transition temperature as measured by TA Instruments DSC Q2000.
⁶Melt flow index measured at 150° C. with a 500 g weight.
⁷Literature values for cellulose acetate.
⁸Melt flow index measured at 150° C. with a 100 g weight.

### Example 7

Some of the adhesive compositions from Tables 1 and 3 were tested for peel adhesion by ASTM 3330/D Method A (180° Peel) after a 24 hour dwell time conditioned at 22° C. and 60% relative humidity. The adhesive strength was measured on stainless steel, glass, and corrugated cardboard and is presented in Table 5.

TABLE 5

| | | | | 180° Peel |
|---|---|---|---|---|
| | | 180° Peel | 180° Peel | Corrugated |
| | | Stainless Steel | Glass | Cardboard |
| | Adhesive | Substrate | Substrate | Substrate |
| | Thickness | 24 hr. | 24 hr. | 24 hr. |
| | (mil) | Dwell Time | Dwell Time | Dwell Time |
| Sample | (mil) | Mean (lbf/in) | Mean (lbf/in) | Mean (lbf/in) |
| PCA-14 | 1.5 | 3.0 | 2.6 | 1.7 |
| PCA-16 | 5 | 1.7 | 2.4 | 1.4 |
| PCA-41 | 1.5 | 0.8 | 1.7 | 1.7 |

### Example 8

A plurality of adhesive samples were prepared by compounding cellulose acetate (CA-4 of Example 6) and a plasticizer in the amounts and compositions detailed in Table 6. The characteristics of the adhesive samples were measured and are reported in Table 6.

TABLE 6

| | | | Melt Flow |
|---|---|---|---|
| | | | Index⁶ |
| Sample | Plasticizer | T_{g}⁵ (° C) | (g/10 min) |
| PCA-17 | 60 wt % diacetin | −69 | 40 |
| PCA-42 | 62 wt % diacetin | −68 | 82 |
| PCA-20 | 57 wt % diacetin and | −66 | 57 |
| | 5 wt % acetylsalicylic acid | | |
| PCA-43 | 50 wt % acetylsalicylic acid | −21 | less than 1 |
| PCA-44 | 60 wt % acetylsalicylic acid | −32 | less than 1 |
| PCA-45 | 33 wt % diacetin and | −57 | 125 |
| | 33 wt % acetylsalicylic acid | | |
| PCA-46 | 49.5 wt % diacetin and | −59 | 100 |
| | 16.5 wt % acetylsalicylic acid | | |
| PCA-47 | 16.5 wt % diacetin and | −48 | 100 |
| | 49.5 wt % acetylsalicylic acid | | |

⁵Glass transition temperature as measured by TA Instruments DSC Q2000.
⁶Melt flow index measured at 150° C. with a 500 g weight.

### Example 9

This example appears to demonstrate the synergistic effect on melt flow index using multiple plasticizers in the adhesives described herein. A plurality of adhesive samples were prepared by compounding cellulose acetate (CA-4 of Example 6) and a plasticizer in the amounts and compositions detailed in Table 7. The characteristics of the adhesive samples were measured and are reported in Table 7.

TABLE 7

| | | | Melt Flow |
|---|---|---|---|
| | | | Index⁶ |
| Sample | Plasticizer | T_{g}⁵ (° C.) | (g/10 min) |
| PCA-17 | 60 wt % diacetin | −69 | 40 |
| PCA-48 | 60 wt % triethylcitrate | −56 | 15 |
| PCA-49 | 30 wt % diacetin and | −61 | 45 |
| | 30 wt % triethylcitrate | | |
| PCA-42 | 62 wt % diacetin | −68 | 82 |
| PCA-79 | 62 wt % imidazole | −50 | less than 1 |
| PCA-51 | 57 wt % diacetin and | −62 | 109 |
| | 5 wt % imidazole | | |

⁵Glass transition temperature as measured by TA Instruments DSC Q2000.
⁶Melt flow index measured at 150° C. with a 500 g weight.

### Example 10

This example appears to demonstrate the use of amines as plasticizers in the adhesives described herein. A plurality of adhesive samples were prepared by compounding cellulose acetate (CA-4 of Example 6) and a plasticizer in the amounts and compositions detailed in Table 8. The characteristics of the adhesive samples were measured and are reported in Table 8.

TABLE 8

| Sample | Plasticizer | T_{g}⁵ (° C.) |
|---|---|---|
| PCA-17 | 60 wt % diacetin | −69 |
| PCA-50 | 60 wt % imidazole | −53 |
| | 57 wt % diacetin and | |
| PCA-51 | 5 wt % imidazole | −62 |
| PCA-52 | 50 wt % ethylene diamine | none |
| | | detected |
| PCA-53 | 50 wt % piperidine | none |
| | | detected |
| PCA-54 | 50 wt % piperazine | −60 |
| PCA-55 | 50 wt % hexanediamine | −65 |

⁵Glass transition temperature as measured by TA Instruments DSC Q2000.

### Example 11

This example appears to demonstrate the effect of tackifiers on the properties of the adhesives described herein. A plurality of adhesive samples were prepared by compounding cellulose acetate (CA-4 of Example 6 or CA-5 (a blend of two cellulose acetates both having a degree of substitution of about 2.3 and each an intrinsic viscosity of about 1.27 dL/g and 1.21 dL/g), a plasticizer, and tackifiers (terpene phenolic resins, SYLVARES™ TP96 and SYLVARES™ TP2040 and rosin esters, SYLVALITE™ RE 100XL, available from Arizona Chemical) in the amounts and compositions detailed in Table 9. The characteristics of the adhesive samples were measured and are reported in Table 9.

TABLE 9

| | | | | | Melt |
|---|---|---|---|---|---|
| | | | | | Flow |
| | | | | | Index |
| | | | | T_{g}⁵ | (g/10 |
| Sample | Cellulose | Plasticizer | Tackifier | (° C.) | min) |
| PCA-56 | CA-4 | 57 wt % | 5 wt % | −68 | 51⁶ |
| | | diacetin | SYLVARES ™ | | |
| | | | TP96 | | |
| PCA-57 | CA-4 | 57 wt % | 5 wt % | −68 | 62⁶ |
| | | diacetin | SYLVARES ™ | | |
| | | | TP2040 | | |
| PCA-58 | CA-5 | 51 wt % | 15 wt % | −66 | 49⁸ |
| | | diacetin | SYLVARES ™ | | |
| | | | TP2040 | | |
| PCA-59 | CA-5 | 57 wt % | 5 wt % | none | 10⁸ |
| | | diacetin | SYLVALITE ™ | detected | |
| | | | RE 100XL | | |
| PCA-60 | CA-5 | 51 wt % | 15 wt % | −62 | 11⁸ |
| | | diacetin | SYLVALITE ™ | | |
| | | | RE 100XL | | |
| PCA-61 | CA-5 | 47.12 wt % | 14.88 wt % | −62 | 5⁸ |
| | | diacetin | SYLVALITE ™ | | |
| | | | RE 100XL | | |
| PCA-62 | CA-5 | 42 wt % | 30 wt % | −61 | 30⁸ |
| | | diacetin | SYLVALITE ™ | | |
| | | | RE 100XL | | |
| PCA-63 | CA-5 | 32.24 wt % | 29.76 wt % | −61 | 32⁶ |
| | | diacetin | SYLVALITE ™ | | |
| | | | RE 100XL | | |

⁵Glass transition temperature as measured by TA Instruments DSC Q2000.
⁶Melt flow index measured at 150° C. with a 500 g weight.
⁸Melt flow index measured at 150° C. with a 100 g weight.

### Example 12

This example appears to demonstrate the effect of nonionic surfactants on the properties of the adhesives described herein. A plurality of adhesive samples were prepared by compounding cellulose acetate (CA-5 of Example 11), a plasticizer, tackifiers, and surfactant (GLYCOMUL® L, sorbitan monolaurate, available from Lonza) in the amounts and compositions detailed in Table 10. The characteristics of the adhesive samples were measured and are reported in Table 10.

TABLE 10

| | | | | | | | | MFI⁸ |
|---|---|---|---|---|---|---|---|---|
| Sample | Cellulose | Plasticizer | | Tackifier | | Surfactant | T_{g}⁵ (° C.) | (g/10 min) |
| PCA-59 | CA-5 | 57 | wt % | 5 | wt % | 0 wt % | none | 10 |
| | | diacetin | | SYLVALITE ™ | | | detected | |
| | | | | RE 100XL | | | | |
| PCA-64 | CA-5 | 43.89 | wt % | 18.8 | wt % | 5 wt % | −65 | 48 |

| | |
|---|---|
| diacetin | SYLVALITE ™ |
| | RE 100XL |

⁵Glass transition temperature as measured by TA Instruments DSC Q2000.
⁸Melt flow index measured at 150° C. with a 100 g weight.

### Example 13

This example appears to demonstrate the effect of cellulosic source on the properties of the adhesives described herein. A plurality of adhesive samples were prepared by compounding cellulose acetate from different cellulosic sources. CA-4 and CA-5 described in Examples 6 and 11, respectively, were prepared with acetate grade cellulose, which has an alpha-cellulose content of greater than 94%. CA-6 was prepared to have similar degree of substitution and molecular weight as CA-4 but with viscose grade cellulose starting material, which has an alpha-cellulose content of about 90% to about 94%. The adhesive formulations and characteristics are provided in Table 11.

TABLE 11

| | | | | T_{g}⁵ | MFI |
|---|---|---|---|---|---|
| Sample | Cellulose | Plasticizer | Tackifier | (° C.) | (g/10 min) |
| PCA-17 | CA-4 | 60 wt % | 0% | −69 | 40⁶ |
| | | diacetin | | | |
| PCA-42 | CA-4 | 62 wt % | 0% | −68 | 82⁶ |
| | | diacetin | | | |
| PCA-65 | CA-6 | 60 wt % | 0% | −67 | 75⁶ |
| | | diacetin | | | |
| PCA-66 | CA-6 | 62 wt % | 0% | −66 | 101⁶ |
| | | diacetin | | | |
| PCA-59 | CA-5 | 57 wt % | 5 wt % | none | 10⁸ |
| | | diacetin | SYLVALITE ™ | detected | |
| | | | RE 100XL | | |
| PCA-60 | CA-5 | 51 wt % | 15 wt% | −62 | 11⁸ |
| | | diacetin | SYLVALITE ™ | | |
| | | | RE 100XL | | |
| PCA-61 | CA-5 | 47.12 | 14.88 wt % | −62 | 5⁸ |
| | | wt % | SYLVALITE ™ | | |
| | | diacetin | RE 100XL | | |
| PCA-67 | CA-6 | 57 wt % | 5 wt % | −72 | 44⁸ |
| | | diacetin | SYLVALITE ™ | | |
| | | | RE 100XL | | |
| PCA-68 | CA-6 | 51 wt % | 15 wt % | | |
| | | diacetin | SYLVALITE ™ | −55 | 37⁸ |
| | | | RE 100XL | | |
| PCA-69 | CA-6 | 47.12 | 14.88 wt % | | |
| | | wt % | SYLVALITE ™ | −66 | 27⁸ |
| | | diacetin | RE 100XL | | |

⁵Glass transition temperature as measured by TA Instruments DSC Q2000.
⁶Melt flow index measured at 150° C. with a 500 g weight.
⁸Melt flow index measured at 150° C. with a 100 g weight.

### Example 14

This example appears to demonstrate the effect of nonionic surfactants on the properties of the adhesives described herein. A plurality of adhesive samples were prepared by compounding cellulose acetate (CA-5 of Example 11), a plasticizer, tackifiers, and surfactant in the amounts and compositions detailed in Table 12. The characteristics of the adhesive samples were measured and are reported in Table 12.

TABLE 12

| | | | | | MFI⁸ |
|---|---|---|---|---|---|
| | | | | T_{g}⁵ | (g/10 |
| Sample | Plasticizer | Tackifier | Surfactant | (° C.) | min) |
| PCA-70 | 37.62 | 25 wt % | 5 wt % | −63 | 31 |
| | wt % | SYLVALITE ™ | Brij L23 (30% | | |
| | diacetin | RE 100XL | (w/v) in H₂O | | |
| PCA-71 | 37.62 | 25 wt % | 5 wt % | | |
| | wt % | SYLVALITE ™ | SIDERCEL SF | −64 | 41 |
| | diacetin | RE 100XL | 140 | | |
| PCA-72 | 37.62 | 25 wt % | 5 wt % | | |
| | wt % | SYLVALITE ™ | TRITON X-100 | −62 | 31 |
| | diacetin | RE 100XL | | | |
| PCA-73 | 37.62 | 25 wt % | 5 wt % | | |
| | wt % | SYLVALITE ™ | POLYFOX PF- | −63 | 17 |
| | diacetin | RE 100XL | 151N | | |
| PCA-74 | 37.62 | 25 wt % | 5 wt % | | |
| | wt % | SYLVALITE ™ | GLYCOSPERSE | −64 | 41 |
| | diacetin | RE 100XL | L-20 KFG | | |
| PCA-75 | 39.60 | 26.4 wt % | 0 wt % | −66 | 11 |
| | wt % | SYLVALITE ™ | | | |
| | diacetin | RE 100XL | | | |

⁵Glass transition temperature as measured by TA Instruments DSC Q2000.
⁸Melt flow index measured at 150° C. with a 100 g weight.

### Example 15

This example appears to demonstrate the ability to produce adhesives with base polymers that include PCA and traditional adhesive polymers (e.g., ethylene vinyl acetate copolymer (“EVA”) and polyvinyl alcohol (“PVOH”)). Interestingly, in these exemplary adhesive compositions, compatibilizers were not required. A plurality of adhesive samples were prepared by compounding cellulose acetate (CA-5 of Example 11), a plasticizer, and an additional polymer in the amounts and compositions detailed in Table 13. The characteristics of the adhesive samples were measured and are reported in Table 13.

TABLE 13

| | | | | T_{g}⁵ | MFI⁸ |
|---|---|---|---|---|---|
| Sample | Cellulose | Plasticizer | Additional Polymer | (° C.) | (g/10 min) |
| PCA-76 | 38% CA-5 | 57% | 5% EVA | −62 | 61 |
| | | diacetin | (28% vinyl acetate) | | |
| PCA-77 | 38% CA-5 | 57% | 5% PVOH | −65 | 40 |
| | | diacetin | (98.4% hydrolysis) | | |
| PCA-78 | 38% CA-5 | 57% | 5% PVOH | −63 | 34 |
| | | diacetin | (88% hydrolysis) | | |

⁵Glass transition temperature as measured by TA Instruments DSC Q2000.
⁸Melt flow index measured at 150° C. with a 100 g weight.

### Example 16

Starch acetate samples were prepared by reacting starch from various sources with acetic acid, acetic anhydride, and phosphoric acid at 85° C. to 115° C. for 2 hours. The samples were then precipitated in water, filtered, washed with water, and then dried overnight at 85° C. The molecular weight and acetyl value were measured and are presented in Table 14.

TABLE 14

| Starch Source | MW (g/mol) | Degree of Substitution |
|---|---|---|
| dent corn 1 | ~16,000 | ~2.9 |
| dent corn 2 | ~26,000 | ~2.8 |
| dent corn 3 | ~10,000 | ~2.8 |
| dent corn 4 | ~3,800 | ~2 |
| waxy rice | ~8,000 | ~3 |
| waxy corn | ~30,000 | ~2.9 |
| tapioca | ~25,000 | ~2.8 |

### Example 17

The dent corn 1 derived starch acetate from Example 16 was compounded with triacetin and a cellulose acetate according to Table 15. The MFI for the samples was measured with a 100 g weight at 125° C. with a 300 sec melt time.

TABLE 15

| | Wt % | Wt % | | |
|---|---|---|---|---|
| | Starch | Cellulose | Wt % | MFI |
| Sample | Acetate | Acetate | Triacetin | (g/10 min) |
| PSE-1 | 40 | 0 | 60 | 400 |
| PSE-2 | 50 | 0 | 50 | 145 |
| PSE-3 | 30 | 10 | 60 | 195 |
| PSE-4 | 20 | 20 | 60 | 91 |
| PSE-5 | 10 | 30 | 60 | 44 |
| PSE-6 | 4 | 36 | 60 | 15 |
| Cellulose | 0 | 40 | 60 | 6 |
| Ester | | | | |
| Control | | | | |

### Example 18

The dent corn 1 derived starch acetate from Example 16 was compounded with triacetin according to Table 3. The MFI for the samples was measured with a 500 g weight at 125° C. with a 300 sec melt time.

TABLE 16

| Sample | Wt % Starch Acetate | Wt % Triacetin | MFI (g/10 min) |
|---|---|---|---|
| PSE-7 | 60 | 40 | 70 |
| PSE-8 | 65 | 35 | 20 |

### Example 19

The dent corn 1 derived and waxy corn derived starch acetates from Example 16 were compounded with triacetin and a cellulose acetate according to Table 17. The MFI for the samples was measured with a 100 g weight at 125° C. with a 300 sec melt time.

TABLE 17

| | | Wt % | | |
|---|---|---|---|---|
| | Wt % Starch | Cellulose | | MFI (g/10 |
| Sample | Acetate | Acetate | Wt % Triacetin | min) |
| PSE-9 | 20 (waxy) | 20 | 60 | >98 |
| PSE-10 | 20 (dent-1) | 20 | 60 | 91 |

### Example 20

The dent corn 1 derived starch acetate from Example 16 was compounded with triacetin and a cellulose acetate and optionally with additional fillers according to Table 18. The MFI for the samples was measured with a 100 g weight at 125° C. with a 300 sec melt time.

TABLE 18

| | Wt % | Wt % | | | |
|---|---|---|---|---|---|
| | Starch | Cellulose | Wt % | Additional Wt % | MFI (g/10 |
| Sample | Acetate | Acetate | Triacetin | Filler | min) |
| PSE-4 | 20 | 20 | 60 | — | 91 |
| PSE-4a | 20 | 20 | 60 | 2% NCC | 80 |
| PSE-3 | 30 | 10 | 60 | — | 195 |
| PSE-3a | 30 | 10 | 60 | 2% NCC | 135 |
| PSE-1 | 40 | — | 60 | — | 400 |
| PSE-1a | 40 | — | 60 | 2% NCC | 275 |
| PSE-2 | 50 | — | 50 | — | 145 |
| PSE-2a | 50 | — | 50 | 2% corn starch | 120 |
| | | | | (underivatized) | |
| PSE-2b | 50 | — | 50 | 5% corn starch | 59 |
| | | | | (underivatized) | |
| PSE-2c | 50 | — | 50 | 2% glass beads | 110 |
| PSE-2d | 50 | — | 50 | 2% NCC | 71 |

### Example 21

The adhesive compositions on Table 18 were tested for peel adhesion by ASTM 3330/D Method A (180° Peel) after a 24 hour dwell time conditioned at 22° C. and 60% relative humidity. The adhesive strength was measured on stainless steel, glass, and corrugated cardboard and is presented in Table 19.

TABLE 19

| | 180° Peel | | |
|---|---|---|---|
| | Corrugated | 180° Peel | |
| | Cardboard | Stainless Steel | 180° Peel Glass |
| | Substrate | Substrate | Substrate |
| Sample | Mean (lbf/in) | Mean (lbf/in) | Mean (lbf/in) |
| PSE-1 | 0.7056 | — | — |
| PSE-2 | 3.9518 | 3.4662 | 4.06 |
| PSE-2a | 4.7394 | 3.6978 | 3.63 |
| PSE-2b | 3.1349 | 3.6658 | — |
| PSE-2c | 4.4970 | 3.9655 | 4.57 |
| PSE-2d | 2.4097 | 3.5591 | — |

### Example 22

Two dent corn-derived starch acetates (SA-1 and SA-2) were compounded with several plasticizers in the amounts and compositions detailed in Tables 20-22. The physical properties of the resultant samples were analyzed via DSC with TA Instruments DSC Q2000. The samples were cycled twice from −90° C. to 200° C. with a ramp rate of 5° C./min. The glass transition temperature, the cold crystallization temperature, and the melting temperature were measured on the second heating cycle. The results for SA-1 and SA-2 are presented in Table 21 and 22, respectively, where “ND” refers to no detectable measurement.

TABLE 20

| Starch Acetate | Degree of | | |
|---|---|---|---|
| Composition | Substitution | MW (g/mol) | Wt % Plasticizer |
| SA-1 | about 2 | 3,800 | 50 |
| SA-2 | about 3 | 16,000 | 50 |

TABLE 21
Adhesive Compositions with SA-1

| | | Cold Crystallization | Melting Temperature |
|---|---|---|---|
| Plasticizer | T_{g} (° C.) | Temperature (° C.) | (° C.) |
| alpha-pinene | 91 | ND | 150 |
| citral | 43 | ND | ND |
| diacetin | −66 | ND | ND |
| | 11 | | |
| dimethyl adipate | −38 | −4 | 8 |
| dibutyl tartrate | −34 | ND | ND |
| PEG 300 | −64 | ND | ND |
| PEG DGE | −53 | ND | ND |
| resorcinol | −28 | ND | ND |
| monoacetate | | | |
| triacetin | −53 | ND | ND |
| | 45 | | |
| tributyl-o-acetyl | 96 | ND | ND |
| citrate | | | |
| tributylphosphate | 14 | ND | ND |
| triethyl citrate | −50 | ND | ND |
| cinnamyl alcohol | −41 | ND | ND |
| eugenol | −51 | ND | ND |

TABLE 22
Adhesive Compositions with SA-2

| | | Cold Crystallization | Melting Temperature |
|---|---|---|---|
| Plasticizer | T_{g} (° C.) | Temperature (° C.) | (° C.) |
| cinnamyl alcohol | −61 | ND | ND |
| | −39 | | |
| | 17 | | |
| citral | 105 | ND | ND |
| diacetin | −63 | ND | ND |
| dibutyl tartrate | −63 | ND | ND |
| | 131 | | |
| dimethyl adipate | −49 | −27 | ND |
| | 130 | | |
| eugenol | −59 | ND | ND |
| glyceryl | −63 | ND | ND |
| trypropionin | | | |
| gamma- | −12 | ND | ND |
| valerolactone | | | |
| propylene | ND | ND | 148 |
| carbonate | | | 172 |
| resorcinol | −30 | ND | ND |
| monoacetate | | | |
| triacetin | −58 | ND | ND |
| triethyl citrate | −57 | ND | ND |

It was observed that alpha-pinene, PEG 300, poly(ethylene glycol) diglycidyl ether (“PEG-DGE”), tributyl phosphate, and tributyl o-acetyl did not fully plasticize the PSE-2 sample.

### Example 23

A plurality of adhesive compositions were prepared by compounding starch acetate sample dent corn 4 from Example 16, cellulose acetate (a degree of substitution of about 2.4 and a MW of about 44,000 g/mol), and triacetin as described in Table 23. The glass transition temperatures of the samples were measured as described in Example 22 and are also presented in Table 23.

TABLE 23

| Wt % Starch | Wt % Cellulose | | |
|---|---|---|---|
| Acetate | Acetate | Wt % Triacetin | T_{g}¹ (° C.) |
| — | 100 | — | 167-207² |
| 100 | — | — | 130 |
| 4 | 36 | 60 | −53 |
| 10 | 30 | 60 | −56 |
| 20 | 20 | 60 | −58 |
| 30 | 10 | 60 | −62 |
| 40 | 0 | 60 | −62 |

¹Glass transition temperature as measured by TA Instruments DSC Q2000.
²Literature values for cellulose acetate.

### Example 24

The 180° peel test on stainless steel substrates was performed with three adhesive compositions with dwell times of 24 hours and 72 hours. Three adhesive compositions were tested for peel adhesion by ASTM 3330/D Method A (180° Peel) after (1) a 24 hour dwell time and (2) a 72 hour dwell time conditioned at 22° C. and 60% relative humidity. The results provided in Table 24 illustrate that the adhesive strength of the adhesive compositions increases over time.

TABLE 24

| | 180° Peel | 180° Peel |
|---|---|---|
| | Stainless Steel | Stainless Steel |
| | Substrate 24 hr. | Substrate 72 hr. |
| | Dwell Time | Dwell Time |
| Sample | Mean (lbf/in) | Mean (lbf/in) |
| PSE-2 | 3.47 | 4.39 |
| PSE-2b | 3.67 | 5.41 |
| PSE-2c | 3.97 | 4.17 |

### Example 25

This example demonstrates the use of various plasticizers (including amines) in the starch derivative adhesives described herein. A starch acetate was prepared to a degree of substitution of about 2.8 to about 2.9 by reacting industrial corn starch with acetic acid, acetic anhydride, and sulfuric acid, then precipitated, filtered, washed with water, and dried overnight at 85° C. The starch acetate was then used in preparing several samples according to Table 25, which also includes the measured glass transition temperatures as described in Example 22.

TABLE 25

| Sample | Wt % Plasticizer | Plasticizer Composition | T_{g} (° C.) |
|---|---|---|---|
| PSE-11 | 62 | 92:8 diacetin to | −65 |
| | | acetylsalicylic acid | |
| PSE-12 | 62 | 92:8 diacetin to butylated | −66 |
| | | hydroxyanisol | |
| PSE-13 | 62 | 92:8 diacetin to butylated | −65 |
| | | hydroxytoluene | |
| PSE-14 | 62 | 92:8 diacetin to benzoic acid | −66 |
| PSE-15 | 62 | 92:8 diacetin to salicylic acid | −72 |
| PSE-16 | 62 | 92:8 diacetin to imidazole | −60 |

### Example 26

This example demonstrates the use of various plasticizers (including amines) in varying amounts in the starch derivative adhesives described herein. The starch acetate from Example 25 was then used in preparing several samples according to Tables 26-28, which also includes the measured glass transition temperatures as described in Example 22.

TABLE 26

| | Wt % Starch | Wt % | Wt % Acetylsalicylic | |
|---|---|---|---|---|
| Sample | Acetate | Diacetin | Acid | T_{g} (° C.) |
| PSE-17 | 40% | 60% | 0% | −65 |
| PSE-18 | 40% | 0% | 60% | −5 |
| PSE-19 | 38% | 57% | 5% | −65 |
| PSE-20 | 34% | 33% | 33% | −58 |
| PSE-21 | 34% | 49.5% | 16.5% | −67 |
| PSE-22 | 34% | 16.5% | 49.5% | −53 |

TABLE 27

| | Wt % Starch | Wt % | | |
|---|---|---|---|---|
| Sample | Acetate | Diacetin | Wt % Triethylcitrate | T_{g} (° C.) |
| PSE-17 | 40% | 60% | 0% | −65 |
| PSE-23 | 40% | 0% | 60% | −59 |
| PSE-24 | 40% | 30% | 30% | −62 |

TABLE 28

| | Wt % Starch | Wt % | | |
|---|---|---|---|---|
| Sample | Acetate | Diacetin | Wt % Imidazole | T_{g} (° C.) |
| PSE-25 | 40% | 0% | 60% | −53 |
| PSE-16 | 38% | 57% | 5% | −60 |

### Example 27

This example demonstrates the effect of tackifiers on the properties of the starch derivative adhesives described herein. The starch acetate from Example 25 was then used in preparing several samples according to Table 29 using tackifiers (terpene phenolic resins, SYLVARES™ TP2040 and rosin esters, SYLCALITE™ RE 100XL, available from Arizona Chemical) which also includes the measured glass transition temperatures as described in Example 22.

TABLE 29

| | Wt % Starch | Wt % | | T_{g} |
|---|---|---|---|---|
| Sample | Acetate | Diacetin | Wt % Tackifier | (° C.) |
| PSE-17 | 40% | 60% | 0% | −65 |
| PSE-26 | 38% | 57% | 5 wt % | −65 |
| | | | SYLVARES ™ TP2040 | |
| PSE-27 | 34% | 51% | 15 wt % | −67 |
| | | | SYLVARES ™ TP2040 | |
| PSE-28 | 38% | 57% | 5 wt % SYLVALITE ™ | −67 |
| | | | RE 100XL | |
| PSE-29 | 34% | 51% | 15 wt % SYLVALITE ™ | −69 |
| | | | RE 100XL | |

### Example 28

This example demonstrates the effect of nonionic surfactants on the properties of the starch derivative adhesives described herein. A plurality of adhesive samples were prepared by compounding starch acetate of Example 25, a plasticizer, tackifiers, and surfactant in the amounts and compositions detailed in Table 30 which also includes the measured glass transition temperatures as described in Example 22.

TABLE 30

| | | | | T_{g} |
|---|---|---|---|---|
| Sample | Plasticizer | Tackifier | Surfactant | (° C.) |
| PSE-30 | 37.62 wt % | 25 wt % | 5 wt % | −64 |
| | diacetin | SYLVALITE ™ | BRIJ L23 | |
| | | RE 100XL | (30% (w/v) in H₂O) | |
| PSE-31 | 37.62 wt % | 25 wt % | 5 wt % | −62 |
| | diacetin | SYLVALITE ™ | SIDERCEL SF 140 | |
| | | RE 100XL | | |
| PSE-32 | 37.62 wt % | 25 wt % | 5 wt % | −64 |
| | diacetin | SYLVALITE ™ | TRITON X-100 | |
| | | RE 100XL | | |
| PSE-33 | 37.62 wt % | 25 wt % | 5 wt % | −67 |
| | diacetin | SYLVALITE ™ | POLYFOX PF-151N | |
| | | RE 100XL | | |
| PSE-34 | 37.62 wt % | 25 wt % | 5 wt % | −57 |
| | diacetin | SYLVALITE ™ | GLYCOSPERSE | |
| | | RE 100XL | L-20 KFG | |

The above examples illustrate the plurality of adhesive compositions that may be produced with plasticized cellulose derivatives, plasticized starch derivatives, or both optionally additives like fillers. Further, these examples illustrate the ability to tailor the characteristics of the adhesive compositions with changes in the components of the adhesive composition and their relative concentrations.

Therefore, this disclosure is well adapted to attain the ends and advantages mentioned as well as those that are inherent therein. The particular embodiments disclosed above are illustrative only, as the embodiments described herein may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the disclosure. The embodiments illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. A drug delivery vehicle comprising:
an intermediate layer comprising a drug disposed between an adhesive and a backing, wherein one of the intermediate layer and the adhesive are formed by a polymeric matrix that comprises a plasticizer and at least one selected from the group consisting of: a cellulose ester, a cellulose ether, a starch ester, a starch ether, and a combination thereof.

2. The drug delivery vehicle of claim 1, wherein the adhesive comprises the polymeric matrix and the plasticizer is in the polymeric matrix at about 15% or greater by weight of the polymeric matrix.

3. The drug delivery vehicle of claim 1, wherein the intermediate layer comprises the polymeric matrix and the polymeric matrix consists essentially of the plasticizer, the drug, and the at least one selected from the group consisting of: a cellulose ester, a cellulose ether, a starch ester, a starch ether, and a combination thereof.

4. The drug delivery vehicle of claim 1, wherein the polymeric matrix comprises the at least one selected from the group consisting of: the cellulose ester, the cellulose ether, the starch ester, the starch ether, and the combination thereof at about 10% to about 80% by weight of the polymeric matrix.

5. The drug delivery vehicle of claim 1, wherein the polymeric matrix further comprises at least one selected from the group consisting of: a polyolefin, an ethylene copolymer, a thermoplastic polyurethane, an acrylic acid polymer, polytetrafluoroethylene, an ethylene vinyl acetate copolymer derivative, a polyester, a polysiloxane, polybutadiene, polyisoprene, poly(methacrylate), poly(methyl methacrylate), a styrene-butadiene-styrene block copolymer, poly(hydroxyethylmethacrylate), poly(vinyl chloride), poly(vinyl acetate), a polyether, a polyacrylonitrile, a polyethylene glycol, polymethylpentene, polybutadiene, polyhydroxy alkanoates, poly(lactic acid), poly(glycolic acid), acrylic acid-based polymers, a methacrylic acid based polymer, a polyanhydride, a polyorthoester, cross-linked poly(vinyl alcohol), neoprene rubber, butyl rubber, polyvinyl acetate phthalate, polyvinyl acetate, shellac, zein, polyethylene oxide, ethylene oxide-propylene oxide copolymers, polyethylene-polypropylene glycol, carbomer, polycarbophil, chitosan, polyvinyl pyrrolidone, poly(vinyl alcohol), a polyethyleneimine, a polyacrylate, a polyacrylamide, a polymethacrylamide, a polyphosphazine, a polyoxazolidine, a polyhydroxyalkylcarboxylic acid, an alginic acid, natural gums, povidone, gelatin, and the like, any derivative thereof, any copolymer thereof, any blend polymer thereof, and combinations thereof.

6. The drug delivery vehicle of claim 1, wherein the polymeric matrix further comprises ethylene vinyl acetate copolymer.

7. The drug delivery vehicle of claim 1, wherein the polymeric matrix consists essentially of the plasticizer and the at least one selected from the group consisting of: the cellulose ester, the cellulose ether, the starch ester, the starch ether, and the combination thereof.

8. A drug delivery vehicle comprising:
a core polymer matrix comprising a drug and encompassed by a skin polymer matrix, wherein the core polymer matrix or the skin polymer matrix comprises a plasticizer and at least one selected from the group consisting of: a cellulose ester, a cellulose ether, a starch ester, a starch ether, and a combination thereof.

9. The drug delivery vehicle of claim 8, wherein the core polymer matrix consists essentially of the plasticizer, the drug, and the at least one selected from the group consisting of: a cellulose ester, a cellulose ether, a starch ester, a starch ether, and a combination thereof.

10. The drug delivery vehicle of claim 8, wherein the polymeric matrix comprises the at least one selected from the group consisting of: the cellulose ester, the cellulose ether, the starch ester, the starch ether, and the combination thereof at about 10% to about 80% by weight of the polymeric matrix.

11. The drug delivery vehicle of claim 8, wherein the polymeric matrix further comprises ethylene vinyl acetate copolymer.

12. The drug delivery vehicle of claim 8, wherein the polymeric matrix consists essentially of the plasticizer and the at least one selected from the group consisting of: the cellulose ester, the cellulose ether, the starch ester, the starch ether, and the combination thereof.

13. The drug delivery vehicle of claim 8 dimensioned for use as an intravaginal ring.

14. The drug delivery vehicle of claim 8 dimensioned for use as a rod-shaped implant.

15. A drug delivery vehicle comprising:
a bandage with a polymer matrix disposed thereon, wherein the polymer matrix comprises a drug, a plasticizer, and at least one selected from the group consisting of: a cellulose ester, a cellulose ether, a starch ester, a starch ether, and a combination thereof.

16. The drug delivery vehicle of claim 15, wherein the polymer matrix is tacky at room temperature.

17. The drug delivery vehicle of claim 15, wherein the plasticizer is in the polymeric matrix at about 15% or greater by weight of the polymeric matrix.

18. The drug delivery vehicle of claim 15, wherein the at least one selected from the group consisting of: the cellulose ester, the cellulose ether, the starch ester, the starch ether, and the combination thereof is at about 10% to about 80% by weight of the polymeric matrix.

19. The drug delivery vehicle of claim 15, wherein the polymeric matrix further comprises ethylene vinyl acetate copolymer.

20. The drug delivery vehicle of claim 15, wherein the polymeric matrix consists essentially of the drug, the plasticizer, and the at least one selected from the group consisting of: the cellulose ester, the cellulose ether, the starch ester, the starch ether, and the combination thereof.
